(19) United States
(12) Patent Application Publication
(10) Pub. No.: US 2026/0224549 A1
(43) Pub. Date: Aug. 6, 2026

(54) COMPOUNDS, COMPOSITIONS, AND METHODS FOR TREATING, AMELIORATING, AND/OR PREVENTING PAIN

(71) Applicant: Mebias Discovery, Inc., NEW HAVEN, CT (US)

(72) Inventors: Brett A. Tounge, Philadelphia, PA (US); Shariff Bayoumy, Watertown, CT (US); Lawrence C. Kuo, Breckenridge, CO (US); Scott Alan Reines, New Hope, PA (US)

(21) Appl. No.: 19/157,249

(22) PCT Filed: Feb. 16, 2024
(86) PCT No.: PCT/US24/16274

§ 371 Date: Aug. 15, 2025

Related U.S. Application Data
(60) Provisional application No. 63/446,701, filed on Feb. 17, 2023.

(51) Int. Cl. A61K 31/4535; A61P 25/04

(52) U.S. Cl. CPC A61K 31/4535; A61P 25/04

(57) ABSTRACT

The disclosure relates to certain substituted 3-dialkylaminomethyl-piperidin-4-yl-benzamides, as well as compositions comprising such compounds. The disclosure provides in one aspect certain 3-(3S,4R)-3-((dimethylamino)methyl)-4-hydroxy-1-(2-(thiophen-3-yl)ethyl)piperidin-4-yl-benzamide diphosphate crystalline solids, as well as compositions comprising such compounds. In certain embodiments, the compounds of the disclosure are useful for treating, ameliorating, and/or preventing pain in a subject in need thereof.

### CROSS-REFERENCE TO RELATED APPLICATIONS

This application claims priority to U.S. Provisional Patent Application No. 63/446,701 filed Feb. 17, 2023, the disclosure of which is incorporated herein by reference in its entirety.

### BACKGROUND

Pain is defined as an unpleasant sensory and emotional experience. Pain, however, can be informative and useful. Nociceptive pain is often indicative of injury (e.g., tissue damage), and such pain typically evokes escape or protective behaviors in animals, including humans. However, inflammation, cellular and neuronal damage and other processes resulting from injury or disease can lead to states of chronic pathological pain. Hyperalgesia is a condition in which enhanced sensitivity to noxious stimuli is present, and thus the perception of pain is exaggerated. Allodynia is a condition in which normally non-noxious stimuli become painful. Persistent or chronic pain, manifested as hyperalgesia and/or allodynia, remains challenging to treat. Chronic pain contributes to over $600 billion worth of healthcare expenditures annually, more than the yearly cost of cancer, heart disease, and diabetes combined. Neuropathic pain affects about 6-10% of the population, and is associated with decreased quality of life and socioeconomic burdens exceeding all other chronic pain disorders. Many patients do not respond to existing therapeutics, have their pain poorly managed (i.e., inadequate relief), or experience relief for an inadequate duration.

The administration of opioids to treat pain is a well-recognized and commonly employed therapy in medicine. Agonists of the Mu opioid receptor (MOR) are the standard analgesic agents to treat acute, severe pain conditions in humans. Unfortunately, tachyphylaxis and tolerance to opioids, and opioid-induced hyperalgesia, often result during the course of therapy. In such patients, increasingly higher doses of opioids are needed to provide an acceptable level of pain relief and, in doing so, the patient is thereby subjected to a higher risk of additional adverse side effects and safety concerns, which include respiratory depression, constipation, nausea and vomiting. Prolonged opioid therapy to treat chronic pain states may subject the patient to develop dependence on opioids, suffer opioid withdrawal on discontinuation of treatment, and some patients may be more susceptible to engage in abuse of these medications. These phenomena present significant clinical challenges for the treatment of pain.

There is a need in the art for novel compounds and/or compositions that can be used to treat, ameliorate, and/or prevent pain and/or reduce or reverse hyperalgesia and allodynia.

In certain embodiments, the compounds and/or compositions should not induce significant (or any at all) respiratory depression, constipation, and/or tolerance, while providing pain relief. The present disclosure addresses these unmet needs.

### BRIEF SUMMARY

The disclosure provides in one aspect a pharmaceutical composition. In certain embodiments, the pharmaceutical composition comprises at least one pharmaceutically acceptable carrier. In certain embodiments, the pharmaceutical composition comprises an amount of 3-(3S, 4R)-3-((dimethylamino)methyl)-4-hydroxy-1-(2-(thiophen-3-yl)ethyl)piperidin-4-yl-benzamide (Compound I) selected from: 20 mg, 40 mg, 60 mg, 80 mg, 100 mg, 120 mg, 140 mg, 160 mg, 180 mg, 200 mg, 220 mg, 240 mg, 260 mg, 280 mg, 300 mg, 320 mg. 340 mg, 360 mg, 380 mg, and 400 mg.

The disclosure provides in one aspect 3-(3S, 4R)-3-((dimethylamino)methyl)-4-hydroxy-1-(2-(thiophen-3-yl)ethyl)piperidin-4-yl-benzamide (Compound I) diphosphate crystalline solid. In certain embodiments, the solid has a X-ray powder diffraction spectrum comprising 2θ values (in degrees) of about: 5.02, 7.04, 15.12, 20.25, 20.88, and 23.0.

The disclosure provides in one aspect 3-(3S, 4R)-3-((dimethylamino)methyl)-4-hydroxy-1-(2-(thiophen-3-yl)ethyl)piperidin-4-yl-benzamide (Compound I) diphosphate crystalline solid. In certain embodiments, the solid has a X-ray powder diffraction spectrum substantially equal to that of FIG. 9A and/or FIG. 12A.

The disclosure provides in one aspect a pharmaceutical composition comprising at least one pharmaceutically acceptable carrier and a solid of the disclosure.

The disclosure provides in one aspect a method of treating, ameliorating, and/or preventing pain in a subject. In certain embodiments, the method comprises administering to the subject a daily amount of 3-(3S, 4R)-3-((dimethylamino)methyl)-4-hydroxy-1-(2-(thiophen-3-yl)ethyl)piperidin-4-yl-benzamide (Compound I) selected from: 20 mg. 40 mg. 60 mg, 80 mg, 100 mg, 120 mg, 140 mg, 160 mg, 180 mg, 200 mg, 220 mg, 240 mg, 260 mg, 280 mg, 300 mg, 320 mg, 340 mg, 360 mg, 380 mg, and 400 mg. In certain embodiments, the Compound I is a solid of the disclosure 7-13 and/or formulated as part of a pharmaceutical composition of the disclosure.

### BRIEF DESCRIPTION OF THE DRAWINGS

For the purpose of illustrating the disclosure, certain embodiments of the disclosure are depicted in the drawings. However, the disclosure is not limited to the precise arrangements and instrumentalities of the embodiments depicted in the drawings.

FIG. 1 illustrates helices of a dynamic signaling GPCR in solution.

FIG. 2 illustrates non-limiting ¹⁹F-labelled protein constructs of the human μ-opioid receptor.

FIG. 3 depicts an isotherm plot based on static vapor sorption experiments.

FIG. 4 depicts a dendrogram of screening samples.

FIG. 5 depicts a 3D cluster plot of free acid screening samples.

FIGS. 6A-6E depict a x-ray diffractogram (FIG. 6A), ¹H-NMR spectrum (FIG. 6B), thermogravimetric analysis (TGA) thermogram (FIG. 6C), kinetic dynamic vapor sorption (DVS) plot (FIG. 6D), and isothermal DVS plot (FIG. 6E) of Compound I free base.

FIGS. 7A-7C depict a x-ray diffractogram (FIG. 7A), ¹H-NMR spectrum (FIG. 7B), and differential scanning calorimetry (DSC) thermogram (FIG. 7C) of Compound I acetate.

FIGS. 8A-8F depict a x-ray diffractogram (FIG. 8A), ¹H-NMR spectrum (FIG. 8B), DSC thermogram (FIG. 8C), TGA thermogram (FIG. 8D), kinetic DVS plot (FIG. 8E), and isothermal DVS plot (FIG. 8F) of Compound I sulfate.

FIGS. 9A-9D depict a x-ray diffractogram (FIG. 9A), ¹H-NMR spectrum (FIG. 9B), DSC thermogram (FIG. 9C), and TGA thermogram (FIG. 9D) of Compound I diphosphate.

FIGS. 10A-IOF depict a x-ray diffractogram (FIG. 10A), ¹H-NMR spectrum (FIG. 10B), DSC thermogram (FIG. 10C), TGA thermogram (FIG. 10D), kinetic DVS plot (FIG. 10E), and isothermal DVS plot (FIG. 10F) of Compound I dibenzoate.

FIGS. 11A-11D depict a x-ray diffractogram (FIG. 11A), ¹H-NMR spectrum (FIG. 11B), DSC thermogram (FIG. 11C), and TGA thermogram (FIG. 11D) of Compound I glycolate.

FIGS. 12A-12F depict a x-ray diffractogram (FIG. 12A), ¹H-NMR spectrum (FIG. 12B), DSC thermogram (FIG. 12C), thermogravimetric analysis (TGA) thermogram (FIG. 12D), kinetic dynamic vapor sorption (DVS) plot (FIG. 12E), and an isothermal DVS plot (FIG. 12F) of Compound I diphosphate (Form A).

FIG. 13 depicts a x-ray diffraction (XRD) overlay of static vapor sorption experiments using Compound I diphosphate (Form A); RH, relative humidity.

FIG. 14 depicts a x-ray diffractogram of Compound I diphosphate (Amorphous Form).

FIG. 15 depicts mean (±SD) Compound I Plasma Concentration-Time Profiles after a Single Oral Dose of Drug Substance.

FIG. 16 depicts mean (±SD) Compound I Plasma Concentration-Time Profiles after a Single Oral Dose of MEB-1170 (Food Effect Treatment Only, 120 mg).

FIG. 17 depicts mean (±SD) Compound II Plasma Concentration-Time Profiles after a Single Oral Dose of MEB-1170.

FIG. 18 depicts Dose Proportionality Assessment for Cₘₐₓ of Compound I.

FIG. 19 depicts Dose Proportionality Assessment for AUC₀₋ₗₐₛₜ of Compound I.

FIG. 20 depicts Dose Proportionality Assessment for AUC₀₋ₜ of Compound I.

FIG. 21 depicts Dose Proportionality Assessment for AUC_{0-inf} of Compound I.

FIG. 22 depicts Food Effect Assessment for Compound I Exposure.

### DETAILED DESCRIPTION

The disclosure relates in one aspect to compounds, and compositions comprising such compounds, that can be used to treat, ameliorate, and/or prevent pain in a subject in need thereof. In certain embodiments, the subject is a mammal. In other embodiments, the mammal is a dog or cat. In yet other embodiments, the mammal is human.

For the more than 25 million Americans suffering chronic pain, the most effective analgesics are opioids. The reliance on opioids to treat pain is a main cause of Opioid Use Disorder (OUD) leading to the current alarming national opioid public health crisis. There are a variety of approaches to curb the epidemic, among which is the development of novel medications. As such, attention in the past three decades has been placed on finding non-addictive small molecules or biologic therapeutics targeting non-opioid receptors for treating pain. These approaches stem from a long-held belief that the analgesia mediated by opioids is permanently wedded to a multitude of adverse effects including respiratory depression, gastrointestinal dysfunction, tolerance, physical dependence, and abuse. Despite this effort, to-date there is no better efficacious analgesic than an opioid for many types of pain.

In certain embodiments, the compounds of the disclosure bind to the mu (p) opioid receptor (MOR). In other embodiments, the compounds of the disclosure activate and/or act as agonists of the mu opioid receptor. In yet other embodiments, upon binding to the MOR, the compounds of the disclosure induce certain secondary messenger signaling manifested as intracellular responses, such as but not limited to G-protein signaling and β-arrestin signaling. In vet other embodiments, upon binding to the MOR, the compounds of the disclosure decrease cyclic adenosine monophosphate (cAMP) levels.

In certain embodiments, upon binding to the MOR, the compounds of the disclosure activate or behave as agonists of the MOR, induce intracellular responses such as but not limited to G-protein signaling and produce a decrease in cAMP.

In certain embodiments, the compounds of the disclosure do not significantly induce secondary messenger signaling, such as, but not limited to, recruitment of, binding to, and/or association with β-arrestins. More specifically, compounds of this disclosure have half-maximal effective concentration (EC₅₀) values for β-arrestin that are higher than approximately 1 micromolar (EC₅₀>1 μM) and/or exhibit β-arrestin activity of less than, or equal to 20% of the maximal response produced by the synthetic opioid compound known as DAMGO ((2S)-2-[[2-[[(2R)-2-[[(2S)-2-Amino-3-(4-hy droxyphenyl)propanoyl]amino]propanoyl]amino]acetyl]-methylamino]-N-(2-hydroxyethyl)-3-phenylpropanamide) as defined by assays known in the art. In certain embodiments, the compounds of the disclosure induce intracellular responses such as G-protein recruitment and binding, such as for the proteins known as Gi, decrease cAMP levels, and do not significantly induce recruitment, binding to, and/or association with β-arrestins.

In certain embodiments, the compounds of the disclosure induce intracellular responses, such as G-protein recruitment, and binding, such as for the proteins known as Gi, produce a decrease in cAMP, and do not significantly induce recruitment, binding to, and/or association with β-arrestins. In yet other embodiments, the compounds of the disclosure are G-protein biased (i.e., signaling pathway-selective) agonists.

In certain embodiments, the compounds of the disclosure provide relief from, and/or alleviate, pain in a subject. In other embodiments, the compounds of the disclosure bind to the MOR in a biased (i.e., signaling pathway-selective) manner, decreasing cAMP levels with minimal or no recruitment, association and/or interaction with β-arrestins.

In certain embodiments, the compounds of the disclosure diminish pain without producing respiratory depression, such as that caused by morphine and other well-known and widely used opioids and narcotics.

In certain embodiments, the compounds of the disclosure diminish pain without producing constipation, such as that caused by morphine and other well-known and widely used opioids and narcotics.

In certain embodiments, the compounds of the disclosure diminish pain without producing nausea, such as that caused by morphine and other well-known and widely used opioids and narcotics.

In certain embodiments, the compounds of the disclosure diminish pain without producing significant (or any at all) tolerance, such as that caused by morphine and other well-known and widely used opioids and narcotics.

In certain embodiments, the compounds of the disclosure diminish pain without producing significant (or any at all) tachyphylaxis, such as that caused by morphine and other well-known and widely used opioids and narcotics.

In certain embodiments, the compounds of the disclosure diminish pain without producing emesis, such as that caused by morphine and other well-known and widely used opioids and narcotics.

In certain embodiments, the compounds of the disclosure diminish pain without producing adverse effects associated with withdrawal, such as that caused by morphine and other well-known and widely used opioids and narcotics.

In certain embodiments, the compounds of the disclosure diminish pain without producing dependence, such as that caused by morphine and other well-known and widely used opioids and narcotics.

In certain embodiments, the compounds of the disclosure diminish pain without producing one or more of the following phenomena: nausea, emesis, constipation, respiratory depression, somnolence, tolerance, tachyphylaxis, dependence and/or addiction.

The disclosure further provides methods of administering a compound of the disclosure to provide pain relief. In certain embodiments, the pain comprises chronic pain. In other embodiments, the pain comprises neuropathic pain. In yet other embodiments, the pain comprises nociceptive pain. In yet other embodiments, the pain comprises hyperalgesia. In yet other embodiments, the pain comprises allodynia.

Prior to the present day understanding of the β-arrestin pathways, many GPCR ligands were developed pursuing optimization of binding to the 7-transmembrane (7-TM) receptor and not surprisingly, many widely used therapeutics are now known to be promiscuous, engaging both G-protein and β-arrestin pathways. For example, morphine, the classic, prototypic MOR agonist, produces analgesia through G-protein signaling. However, this effect is therapeutically “offset” by β-arrestin driven effects that produce receptor internalization and desensitization, which result in tolerance that necessitates increasing doses to maintain analgesic effect. In addition, β-arrestin driven signaling leads to the production of additional on-target adverse effects such as constipation and respiratory depression.

Mice lacking β-arrestin-2 exhibit enhanced and prolonged analgesia with very little tolerance, as well as attenuated respiratory depression and acute constipation, as compared to wild type. This finding suggests that agonists that selectively activate G-protein signaling, but are devoid of 3-arrestin-mediated effects, can provide morphine-like analgesia without the classic adverse effect profile. The biased MOR agonist, oliceridine or N-[(3-methoxy thiophen-2-yl)methyl]-2-[(9R)-9-pyridin-2-yl-6-oxaspiro[4.5]decan-9-yl]ethanamine, which is selective for G-protein signaling (EC₅₀=8 nM, Eₘₐₓ=83% vs morphine) with only minor β-arrestin recruitment (14% Eₘₐₓ) in cell-based assays, exhibited decreased on-target adverse effects (such as nausea, and decrease in respiratory drive) in healthy volunteers and produced a higher level of analgesia in patients following bunionectomy. This work demonstrates the power of biased ligands for GPCR signaling, which maintain/enhance desired therapeutic benefits while removing adverse side effects.

In certain embodiments, the ability of a compound to activate G-protein or β-arrestin is not an on/off switch for a given pathway, but rather a continuous function. To capture this, each compound is measured for activity in cell assays that are linked to β-arrestin recruitment and G-protein signaling. By comparing the activity between these two measures (accounting for both EC₅₀ and maximal response reached, Eₘₐₓ) a relative activity (RA) value is determined.

$$Δ RA = LOG ( \frac{E {MAX}_{β - Arr}}{EC {50}_{β - Arr}} ) - LOG ( \frac{E {MAX}_{G - Protein}}{EC {50}_{G - Protein}} ) .$$

In the case of MOR, it is desirable to have high activation of the G-protein pathway while reducing activation of β-arrestin. In the case of the formula above, this means a lower RA value.

In one aspect, the present disclosure contemplates Compound I, also known as 3-(3S, 4R)-3-((dimethylamino)methyl)-4-hydroxy-1-(2-(thiophen-3-yl)ethyl)piperidin-4-yl-benzamide, or a therapeutically effective salt and/or solvate thereof:

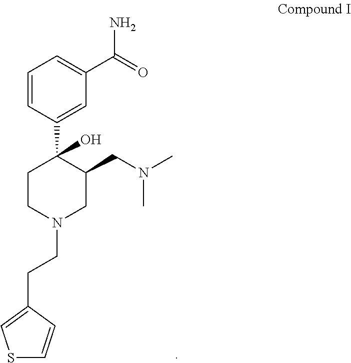 [SMILES: CN(C)C[C@H]1CN(CCc2ccsc2)CC[C@]1(O)c1cccc(C(N)=O)c1]

In one aspect, the present disclosure contemplates Compound II, also known as 3-(3S, 4R)-3-((methylamino)methyl)-4-hydroxy-1-(2-(thiophen-3-yl)ethyl)piperidin-4-yl-benzamide, or a therapeutically effective salt and/or solvate thereof:

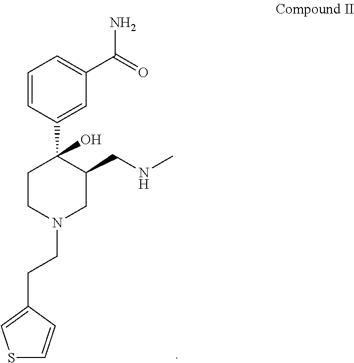 [SMILES: CNC[C@H]1CN(CCc2ccsc2)CC[C@]1(O)c1cccc(C(N)=O)c1]

Compound I was found to be an extremely cell-signaling-pathway-selective μ-opioid agonist. Using a ¹⁹F-protein nuclear magnetic resonance (NMR) platform (¹⁹Fluid) to detect G-Protein coupled receptor (GPCR) solution conformations that regulate pathway selectivity, one can differentiate agonists that are extremely selective in triggering G-protein signaling over β-arrestin signaling.

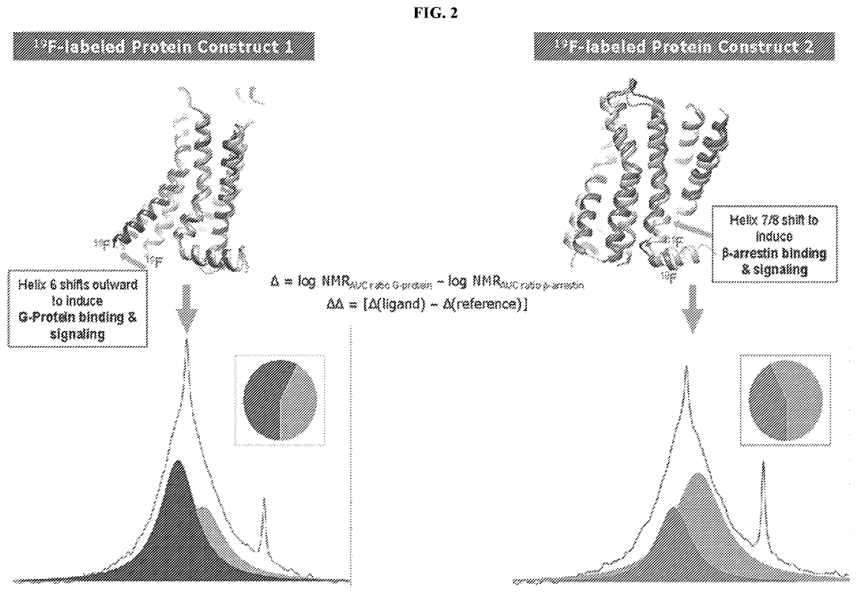

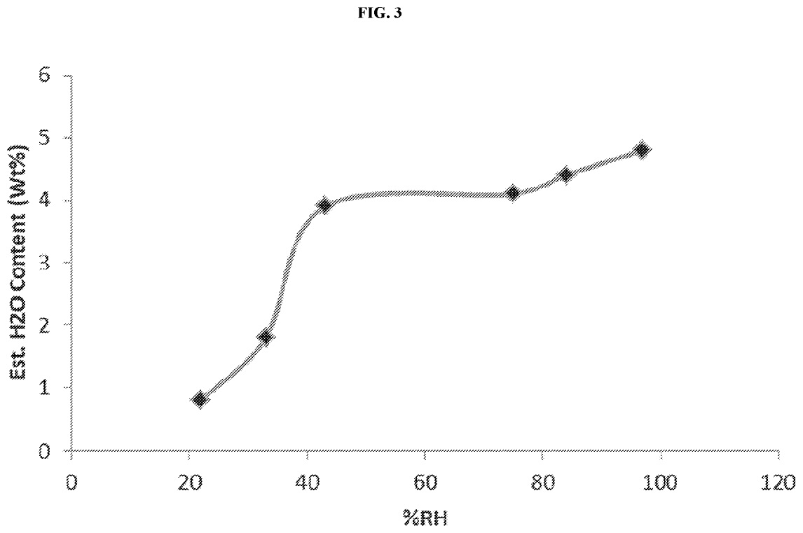

In this ¹⁹Fluid platform approach (FIG. 2), two ¹⁹F-labelled protein constructs of the human μ-opioid receptor were used—one to detect G-protein binding conformation and the other β-arrestin binding conformation. Applying ¹⁹F NMR, one can measure the quantity of G-protein signaling conformation, as well as that of β-arrestin signaling conformation, of the μ-opioid receptor as induced by an agonist. The high level of pathway selectivity detectable by ¹⁹F protein NMR far exceed that can be detected in common cell-based assays.

The antinociceptive effect of Compound I was found to be mediated exclusively by μ-opioid receptor. To separate efficacy from adverse effects for an μ-opioid receptor agonist, signally pathway bias has been introduced into Compound I, such that Compound I displayed no abuse liability in preclinical models including Conditioned Place Preference, Drug Self-Administration, Drug Discrimination, and only minimal Withdrawal symptoms. Most critically, Compound I caused no respiratory depression or other opiate-based adverse effects at exposures far above the ED₅₀ in rats.

Compound I has demonstrated in preclinical models the potential to provide pain relief comparable to Schedule 2 opiates, while greatly reducing or even eliminating the major respiratory and addictive adverse effect liabilities of those products. Compound I thus has the potential to provide opiate-level pain relief without introducing opiate-related adverse effects. It may further address the devastating problem of opioid abuse, overdose, and related death that is currently a major public health problem in the United States.

### Definitions

As used herein, each of the following terms has the meaning associated with it in this section.

Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, separation science, and organic chemistry are those well-known and commonly employed in the art.

As used herein, the articles “a” and “an” refer to one or to more than one (i.e. to at least one) of the grammatical object of the article. By way of example, “an element” means one element or more than one element.

As used herein, the term “about” is understood by persons of ordinary skill in the art and varies to some extent on the context in which it is used. As used herein when referring to a measurable value such as an amount, a temporal duration, and the like, the term “about” is meant to encompass variations of 20% or +10%, more preferably +5%, even more preferably 10%, and still more preferably +0.1% from the specified value, as such variations are appropriate to perform the disclosed methods.

In one aspect, the terms “co-administered” and “co-administration” as relating to a subject refer to administering to the subject a compound of the disclosure or salt thereof along with a compound that may also treat any disease or disorder contemplated herein and/or with a compound that is useful in treating other medical conditions but which in themselves may cause or facilitate any disease or disorder contemplated herein. In certain embodiments, the co-administered compounds are administered separately, or in any kind of combination as part of a single therapeutic approach. The co-administered compound may be formulated in any kind of combinations as mixtures of solids and liquids under a variety of solid, gel, and liquid formulations, and as a solution.

As used herein, “Compound I” or “MEB-1170” or “MEB1170” or “MEB 1170” refers to 3-(3S,4R)-3-((dimethylamino)methyl)-4-hydroxy-1-(2-(thiophen-3-yl)ethyl)piperidin-4-yl-benzamide, or a therapeutically effective salt and/or solvate thereof.

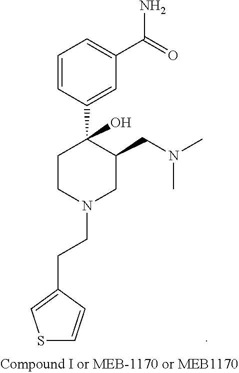 [SMILES: CN(C)C[C@H]1CN(CCc2ccsc2)CC[C@]1(O)c1cccc(C(N)=O)c1]

As used herein. “Compound II” or “MEB-373” or “MEB373” or “MEB 373” refers to 3-(3S,4R)-3-((methylamino)methyl)-4-hydroxy-1-(2-(thiophen-3-yl)ethyl)piperidin-4-yl-benzamide, or a therapeutically effective salt and/or solvate thereof:

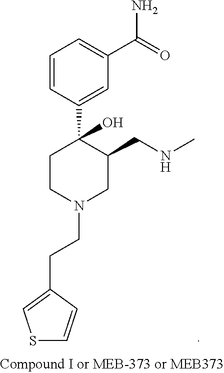 [SMILES: CNC[C@H]1CN(CCc2ccsc2)CC[C@]1(O)c1cccc(C(N)=O)c1]

As used herein, a “disease” is a state of health of a subject wherein the subject cannot maintain homeostasis, and wherein if the disease is not ameliorated then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

As used herein, the term “ED₅₀” refers to the effective dose of a formulation that produces 50% of the maximal effect in subjects that are administered that formulation.

As used herein, an “effective amount,” “therapeutically effective amount” or “pharmaceutically effective amount” of a compound is that amount of compound that is sufficient to provide a beneficial effect to the subject to which the compound is administered.

“Instructional material,” as that term is used herein, includes a publication, a recording, a diagram, or any other medium of expression that can be used to communicate the usefulness of the composition and/or compound of the disclosure in a kit. The instructional material of the kit may, for example, be affixed to a container that contains the compound and/or composition of the disclosure or be shipped together with a container that contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

As used herein, the term “pharmaceutical composition” or “composition” refers to a mixture of at least one compound useful within the disclosure with a pharmaceutically acceptable carrier. The pharmaceutical composition facilitates administration of the compound to a subject.

As used herein, the term “pharmaceutically acceptable” refers to a material, such as a carrier or diluent, which does not abrogate the biological activity or properties of the compound useful within the disclosure, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the subject.

Some examples of materials that may serve as pharmaceutically acceptable carriers include: sugars, such as lactose, glucose and sucrose; starches, such as com starch and potato starch; cellulose, and its derivatives, such as sodium carboxymethyl cellulose, ethyl cellulose and cellulose acetate: powdered tragacanth; malt; gelatin; talc: excipients, such as cocoa butter and suppository waxes; oils, such as peanut oil, cottonseed oil, safflower oil, sesame oil, olive oil, corn oil and soybean oil: glycols, such as propylene glycol; polyols, such as glycerin, sorbitol, mannitol and polyethylene glycol: esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the subject. Supplementary active compounds may also be incorporated into the compositions. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

The term “prevent,” “preventing” or “prevention,” as used herein, means avoiding or delaying the onset of symptoms associated with a disease or condition in a subject that has not developed such symptoms at the time the administering of an agent or compound commences. Disease, condition and disorder are used interchangeably herein.

By the term “specifically bind” or “specifically binds,” as used herein, is meant that a first molecule preferentially binds to a second molecule (e.g., a particular receptor or enzyme), but does not necessarily bind only to that second molecule.

As used herein, a “subject” may be a human or non-human mammal or a bird. Non-human mammals include, for example, livestock and pets, such as ovine, bovine, porcine, canine, feline and murine mammals. In certain embodiments, the subject is human.

The term “treat,” “treating” or “treatment,” as used herein, means reducing the frequency or severity with which symptoms of a disease or condition are experienced by a subject by virtue of administering an agent or compound to the subject.

The following abbreviations are used herein: β-arrestin, beta-arrestin-2 (protein); cAMP, cyclic adenosine monophosphate; EC₅₀, half-maximal (50%) effective concentration; E_{MAX}, maximal response; GPCRs, G-protein coupled receptors; MOR, mu opioid receptor; RA, relative activity.

Throughout this disclosure, various aspects of the disclosure may be presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compounds and Compositions

The present disclosure contemplates certain compounds described herein, and/or a salt, solvate, racemate, enantiomer, racemic diastereoisomer, single diastereoisomer, and/or tautomer thereof.

In one aspect, the present disclosure contemplates Compound I, also known as 3-(3S,4R)-3-((dimethylamino)methyl)-4-hydroxy-1-(2-(thiophen-3-yl)ethyl)piperidin-4-vl-benzamide, or a therapeutically effective salt and/or solvate thereof:

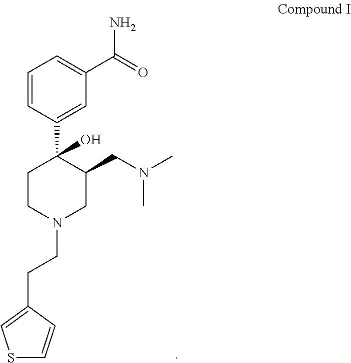 [SMILES: CN(C)C[C@H]1CN(CCc2ccsc2)CC[C@]1(O)c1cccc(C(N)=O)c1]

In one aspect, the present disclosure contemplates Compound I bisphosphate (also known as Compound I diphosphate), also known as 3-(3S,4R)-3-((dimethylamino)methyl)-4-hydroxy-1-(2-(thiophen-3-yl)ethyl)piperidin-4-yl-benzamide bisphosphate, or a therapeutically effective salt and/or solvate thereof:

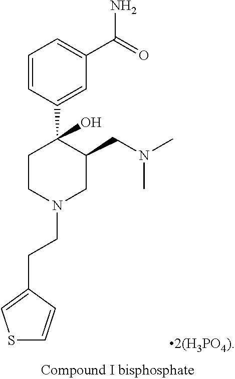 [SMILES: CN(C)C[C@H]1CN(CCc2ccsc2)CC[C@]1(O)c1cccc(C(N)=O)c1]

In one aspect, the present disclosure contemplates Compound II, or 3-(3S,4R)-3-((methylamino)methyl)-4-hydroxy-1-(2-(thiophen-3-yl)ethyl)piperidin-4-yl-benzamide, or a therapeutically effective salt and/or solvate thereof.

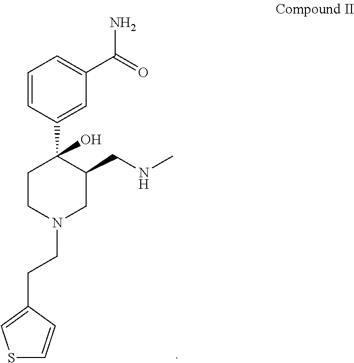 [SMILES: CNC[C@H]1CN(CCc2ccsc2)CC[C@]1(O)c1cccc(C(N)=O)c1]

The disclosure provides pharmaceutical compositions comprising certain amounts of Compound I. The disclosure further provides pharmaceutical compositions comprising certain amounts of Compound I bisphosphate.

In certain embodiments, the disclosure provides a pharmaceutical composition comprising 20 mg Compound I. In certain embodiments, the disclosure provides a pharmaceutical composition comprising 40 mg Compound I. In certain embodiments, the disclosure provides a pharmaceutical composition comprising 60 mg Compound I. In certain embodiments, the disclosure provides a pharmaceutical composition comprising 80 mg Compound I. In certain embodiments, the disclosure provides a pharmaceutical composition comprising 100 mg Compound I. In certain embodiments, the disclosure provides a pharmaceutical composition comprising 120 mg Compound I. In certain embodiments, the disclosure provides a pharmaceutical composition comprising 140 mg Compound I. In certain embodiments, the disclosure provides a pharmaceutical composition comprising 160 mg Compound I. In certain embodiments, the disclosure provides a pharmaceutical composition comprising 180 mg Compound I. In certain embodiments, the disclosure provides a pharmaceutical composition comprising 200 mg Compound I. In certain embodiments, the disclosure provides a pharmaceutical composition comprising 220 mg Compound I. In certain embodiments, the disclosure provides a pharmaceutical composition comprising 240 mg Compound I. In certain embodiments, the disclosure provides a pharmaceutical composition comprising 260 mg Compound I. In certain embodiments, the disclosure provides a pharmaceutical composition comprising 280 mg Compound I. In certain embodiments, the disclosure provides a pharmaceutical composition comprising 300 mg Compound I. In certain embodiments, the disclosure provides a pharmaceutical composition comprising 320 mg Compound I. In certain embodiments, the disclosure provides a pharmaceutical composition comprising 340 mg Compound I. In certain embodiments, the disclosure provides a pharmaceutical composition comprising 360 mg Compound I. In certain embodiments, the disclosure provides a pharmaceutical composition comprising 380 mg Compound I. In certain embodiments, the disclosure provides a pharmaceutical composition comprising 400 mg Compound I.

In certain embodiments, the pharmaceutical composition further comprises at least one pharmaceutically acceptable carrier or excipient.

In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereoisomers. Resolution of compounds and isomers thereof is achieved by any means including, by way of non-limiting example, chemical processes, enzymatic processes, fractional crystallization, distillation, and chromatography. All possible stereochemical configurations of a given compound containing chiral center(s) are contemplated. All possible mixtures enriched with a particular enantiomer or diastereoisomer(s) are contemplated. All pure individual enantiomers or diastereoisomers are contemplated.

The methods and formulations described herein include the use of N-oxides (if appropriate), crystalline forms (also known as polymorphs), solvates, amorphous phases, and/or pharmaceutically acceptable salts of compounds having the structure of any compound of the disclosure, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compounds of the disclosure may exist as tautomers. “Tautomerization” is a form of isomerization involving the migration of a proton accompanied by changes in bond order, often the interchange of a single bond with an adjacent double bond. Where tautomerization is possible, (e.g., in solution), a chemical equilibrium of tautomers can be reached. One well known example of tautomerization is between a ketone and its corresponding enol. Heterocycles may form tautomers such as the interconversion of pyrrolidinone and hydroxypyrrole. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compounds of the disclosure is susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

Compounds of the disclosure can in certain embodiments form acids or bases. In certain embodiments, the disclosure contemplates acid addition salts. In other embodiments, the disclosure contemplates base addition salts. In vet other embodiments, the disclosure contemplates pharmaceutically acceptable acid addition salts. In yet other embodiments, the disclosure contemplates pharmaceutically acceptable base addition salts. Pharmaceutically acceptable salts refer to salts of those bases or acids that are not toxic or otherwise biologically undesirable.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds of the disclosure include, for example, metallic salts including alkali metal, alkaline earth metal and transition metal salts such as, for example, calcium, magnesium, potassium, sodium, lithium and copper, iron and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons. 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

In certain embodiments, compounds of the disclosure can be prepared according to methods known in the art, such as those outlined in WO 2018/045229, US 2019/0194178 A1, WO 2018/136546, and US 2019/0337901 A1, all of which are incorporatde herein in their entireties by reference.

Administration/Dosage/Formulations The disclosure also encompasses pharmaceutical compositions and methods of their use. These pharmaceutical compositions may comprise an active ingredient (which can be one or more compounds of the disclosure, or pharmaceutically acceptable salts thereof) optionally in combination with one or more pharmaceutically acceptable agents. The compositions set forth herein can be used alone or in combination with additional compounds to produce additive, complementary, or synergistic effects.

The regimen of administration may affect what constitutes an effective amount. The therapeutic formulations may be administered to the subject either prior to or after the onset of a disease or disorder contemplated herein. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, preferably a mammal, more preferably a human, may be carried out using known procedures, at dosages and for periods of time effective to treat a disease or disorder contemplated herein. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated herein. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the disclosure is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect, and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder contemplated herein.

In certain embodiments, the compositions of the disclosure are formulated using one or more pharmaceutically acceptable excipients or carriers. In certain embodiments, the pharmaceutical compositions of the disclosure comprise a therapeutically effective amount of a compound of the disclosure and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin.

In certain embodiments, the compositions of the disclosure are administered to the patient in dosages that range from one to five times per day or more. In other embodiments, the compositions of the disclosure are administered to the patient in range of dosages that include, but are not limited to, once every day, every two, days, every three days to once a week, and once every two weeks. It is readily apparent to one skilled in the art that the frequency of administration of the various combination compositions of the disclosure varies from individual to individual depending on many factors including, but not limited to, age, disease or disorder to be treated, gender, overall health, and other factors. Thus, the disclosure should not be construed to be limited to any particular dosage regime and the precise dosage and composition to be administered to any patient is determined by the attending physical taking all other factors about the patient into account.

Compounds of the disclosure for administration may be in the range of from about 1 μg to about 10,000 mg, about 20 μg to about 9,500 mg, about 40 μg to about 9.000 mg, about 75 g to about 8,500 mg, about 150 μg to about 7,500 mg, about 200 μg to about 7,000 mg, about 350 μg to about 6,000 mg, about 500 μg to about 5.000 mg, about 750 μg to about 4,000 mg, about 1 mg to about 3,000 mg, about 10 mg to about 2.500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments there between.

In certain embodiments, the dose of a compound of the disclosure is from about 1 mg and about 2,500 mg. In other embodiments, a dose of a compound of the disclosure used in compositions described herein is less than about 10.000 mg, or less than about 8,000 mg, or less than about 6,000 mg, or less than about 5.000 mg, or less than about 3.000 mg, or less than about 2,000 mg, or less than about 1,000 mg, or less than about 500 mg, or less than about 200 mg, or less than about 50 mg. Similarly, in other embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a disease or disorder contemplated herein.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the disclosure include oral, nasal, rectal, intravaginal, parenteral, buccal, sublingual or topical. The compounds for use in the disclosure may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the disclosure may be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e. drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) melt.

The present disclosure also includes a multi-layer tablet comprising a layer providing for the delayed release of one or more compounds of the disclosure, and a further layer providing for the immediate release of a medication for treatment of diseases or disorders. Using a wax/pH-sensitive polymer mix, a gastric insoluble composition may be obtained in which the active ingredient is entrapped, ensuring its delayed release.

### Parenteral Administration

For parenteral administration, the compounds of the disclosure may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this disclosure include dosage forms as described in U.S. Pat. Nos. 6,340,475; 6,488,962; 6,451,808; 5,972,389; 5,582,837; and 5,007,790. Additional dosage forms of this disclosure also include dosage forms as described in U.S. Patent Applications Nos. 20030147952; 20030104062; 20030104053; 20030044466; 20030039688; and 20020051820. Additional dosage forms of this disclosure also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release that is longer that the same amount of agent administered in bolus form.

For sustained release, the compounds may be formulated with a suitable polymer or hydrophobic material that provides sustained release properties to the compounds. As such, the compounds for use the method of the disclosure may be administered in the form of microparticles, for example, by injection or in the form of wafers or discs by implantation.

In certain embodiments, the compounds of the disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present disclosure depends on the age, sex and weight of the patient, the current medical condition of the patient and the progression of a disease or disorder contemplated herein in the patient being treated. The skilled artisan is able to determine appropriate dosages depending on these and other factors.

A suitable dose of a compound of the present disclosure may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the disclosure is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days. 150 days, 180 days, 200 days, 250 days, 280 days, 300 days. 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the viral load, to a level at which the improved disease is retained. In certain embodiments, patients require intermittent treatment on a long-term basis upon any recurrence of symptoms and/or infection.

The compounds for use in the method of the disclosure may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents are considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

### EXPERIMENTAL EXAMPLES

The disclosure is now described with reference to the following Examples. These Examples are provided for the purpose of illustration only and the disclosure should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present disclosure and practice the claimed methods. The following working examples therefore, point out specific embodiments of the present disclosure, and are not to be construed as limiting in any way the remainder of the disclosure.

### Example 1: Phase I, Double-Blind, Placebo-Controlled, Single and Multiple Oral Ascending Dose Study to Assess Safety, Tolerability, Pharmacokinetics, and Pharmacodynamics of Compound I in Healthy Subjects

Compound I is a signaling pathway-selective μ-opioid agonist. Compound I bisphosphate is a crystalline white to off-white solid and can be encapsulated in size 2 capsules for oral dosing. In certain non-limiting embodiments, dosage strengths are 20 mg and 40 mg capsules. Placebo capsules mimicking the active capsules in appearance can be utilized for the purpose of blinding.

In one aspect, the present study aims to determine the safety and tolerability of single and multiple ascending oral doses of Compound I in healthy subjects.

In certain embodiments, the present study aims to determine the single and multiple oral dose pharmacokinetic profiles of Compound I and the primary metabolite, Compound II, in healthy subjects.

In certain embodiments, the present study aims to determine the effect of food on the pharmacokinetic (PK) profile of a single oral dose of Compound I in healthy subjects.

In certain embodiments, the present study aims to assess the pharmacodynamic (PD) response following single and multiple oral doses of Compound I.

This is a double-blind, placebo-controlled, single and multiple oral dose study conducted in two parts:

### Part A: SAD+FE

This is a randomised, double-blind, placebo-controlled, single ascending dose (SAD) design to assess the safety, tolerability, and PK profile of multiple doses of Compound I administered under fasting conditions in healthy subjects. Up to 5 dosing cohorts are planned.

Part A comprises a single ascending dose (SAD), sequential cohort study, incorporating a food effect (FE) evaluation. Up to 40 subjects are studied in 5 cohorts (Cohorts A1 to A5), each cohort consisting of 8 subjects (6 treated with Compound 1, 2 treated with placebo).

Subjects in Cohorts A1, A2, A4 and A5 participate in 1 treatment period only, residing at the CRU from Day −1 (the day before dosing) to Day 3 (48 hours post-dose). Subjects in Cohort A3 participate in 2 treatment periods (once in fasted state, once in fed state) separated by a minimum of 6 days. All subjects return for a poststudy visit approximately on Day 5 (+2 days) for a final safety assessment.

Each Cohort includes sentinel dosing such that two subjects (one active and one placebo) are dosed at least 48 hours before the remaining subjects in the cohort. Continuation to dose the remaining subjects is at the investigator's discretion.

In each of Cohorts A1 to A5, 6 subjects receive Compound I and 2 receive placebo. All doses are administered in accordance with a randomization schedule in the fasted state in the morning of Day 1, except for Cohort A3 Treatment Period 2 where Compound I is given 30 minutes after start of a high fat breakfast. Each subject in Cohorts A1. A2, A4 and A5 receives only a single dose of Compound I or placebo during the study.

Subjects in Cohort A3 participate in a 2-period treatment design in which they are assessed for both the single-dose of Compound I in a Fed and in a Fasted condition. Subjects receive the same treatment (i.e., either Compound I or placebo) in both Period I and Period 2, and thus subjects receive either two single doses of Compound I or two single doses of placebo during the study. Fasting state assessments occur in Period 1 and Fed state assessments in Period 2. It is planned that an approximate 7-day washout is needed between the Fast and Fed dosing periods. PD assessments are not performed for the food effect Cohort A3.

### Study Design:

### SAD Assessments:

Safety/tolerability throughout study
Physical examination, vital signs, clinical laboratory findings, and ECG
PK concentrations
PD assessments (pupillometry, capnography, oximetry, cold pressor testing)

Following the completion of each cohort, a safety and tolerability review are conducted by the Safety Review Committee (SRC; see elsewhere herein) prior to proceeding to the next cohort. Based on this review, a decision is made to continue the study as planned, repeat the same dose in another Cohort, assess a lower dose, add an intermediate dose, or terminate the study. Additionally, if no dose limiting toxicities are seen, further cohorts at higher doses may be added.

### Part B: MAD

Part B comprises a multiple ascending dose (MAD), sequential cohort study. This part is initiated after the first three SAD cohorts have been fully evaluated for safety and tolerability and the SRC has concluded that the MAD portion may commence. Up to 32 subjects are studied in 4 cohorts (Cohorts B1 to B4), each cohort consisting of 8 subjects.

In each of Cohorts B1 to B4, 6 subjects receive Compound 1 and 2 receive placebo. Once-daily dosing occurs on Days 1 to 7, inclusive, for all subjects. Each subject participates in 1 treatment period only, residing at the CRU from the evening of Day −1 (the day before dosing) until the morning of Day 9 (48 hours after the final dose on Day 7).

All subjects return for a poststudy visit on Day 15 (±2 days) after their final dose for a final safety assessment.

Dose levels to be studied are determined following review of data from Part A. Following completion of each cohort in Part B, a safety and tolerability review is conducted by the SRC prior to proceeding to the next cohort (see elsewhere herein). Based on this review, a decision is made to continue the study as planned, repeat the same dose in another Cohort, assess a lower dose, add an intermediate dose or terminate the study. Additionally, if no dose limiting toxicities are seen, further cohorts at higher doses may be added.

### MAD Assessments:

Safety/tolerability throughout study
Physical examinations, vital signs, clinical laboratory findings, and ECG
PK concentration (see Schedule of Assessments for details)
PD assessments (pupillometry, capnography, cold pressor testing, oximetry)

### Number of Subjects:

Part A: SAD+FE: Up to 40 subjects are studied in 5 cohorts (Cohorts A1 to A5), each cohort consisting of 8 subjects.
Part B: MAD: Up to 32 subjects are studied in 4 cohorts (Cohorts B1 to B4), each cohort consisting of 8 subjects.

### Diagnosis and Main Eligibility Criteria:

1. Healthy males and females as determined by medical history, physical examination, laboratory, and ECG findings
2. Age—18 to 55 years inclusive
3. BMI—18 to 32 kg/m² inclusive
4. Ethnic origin—Any
5. Smoking status—Current Non-smokers
6. Negative Covid antigen or PCR test
7. Ability to tolerate cold pressor test

### Test Product, Dose and Mode of Administration:

In certain embodiments, Compound I is provided as 20 mg and 40 mg capsules by oral administration.

Projected doses in SAD cohorts: 20 mg. 60 mg. 120 mg, 200 mg, 400 mg.

Projected doses in MAD cohorts: 60 mg, 100 mg, 200 mg, 400 mg. Based on the ongoing review of the preliminary safety and tolerability data, as well as available preliminary PK data from previous groups, subsequent planned doses may be adjusted downward or upward, delayed, not administered, or repeated.

If 200 mg dose is well-tolerated, dose has final escalation to 400 mg. If 400 mg dose not well-tolerated, a 300 mg dose group may be tested.

If 200 mg dose is not well-tolerated, a 300 mg dose may be substituted for the 400 mg dose.

For all cohorts EXCEPT the food-effect cohort, morning doses are administered after completing an overnight fast of at least 8 hours. Standard breakfast is available approximately 90 minutes following morning dosing. Evening doses, if determined to be necessary based on SAD PK findings, are included by a protocol amendment. Such doses are administered 12 hours after the morning dose and at least 1 hour after completing the evening meal. However, dosing and food considerations (fed or fasted) for the MAD portion of the study may change pending outcomes from the SAD and food-effect assessments.

For the food-effect cohort, dosing is administered with either a high-fat breakfast or in a fasting state. PD assessments is not performed for the food effect Cohort A3.

### Duration of Treatment:

Part A: SAD+FE: Each subject participates in 1 treatment period only, residing at the CRU from Day −1 (the day before dosing) to Day 3 (48 hours post-dose), except for Cohort A3, where each subject participates in 2 treatment periods, separated by a minimum of 6 days. Subjects return for a poststudy visit on Day 5 (+2 days) for a final safety assessment.
Part B: MAD: Each subject participates in 1 treatment period only, residing at the CRU from the evening of Day −1 (the day before dosing) until the morning of Day 9 (48 hours after the final dose on Day 7). Subjects will return for a poststudy visit on Day 15 (±2 days) for a final safety assessment.

### Reference Therapy, Dose and Mode of Administration:

### Matching Placebo Capsule Administered Orally.

For all cohorts EXCEPT the food-effect cohort, morning doses are administered after completing an overnight fast of at least 8 hours. Standard breakfast is available approximately 90 minutes following morning dosing. Evening doses, if included, are administered 12 hours after the morning dose and at least 1 hour after completing the evening meal. However, dosing and food considerations (fed or fasted) for the MAD portion of the study may change pending outcomes from the SAD and food-effect assessments.

For the food-effect cohort, dosing is administered with either a high-fat breakfast or in a fasting state.

### Criteria for Evaluation:

### Primary Assessments:

The safety assessments include clinical laboratory assessments (chemistry, hematology, urine), ECGs, physical examinations, vital signs, adverse events and suicidality assessment (Part B multiple dose only).

### Secondary Assessments:

### Single Dose PK:

Plasma Compound I concentrations and those of the primary metabolite Compound II is measured after a single oral dose of Compound I at pre-dose and at 0.5, 1, 1.5, 2, 2.5, 3, 3.5, 4, 6, 8, 12, 16, 24, 36 and 48 hours post-dose

### Multiple Dose PK:

Following the initial dose, plasma Compound I concentrations and those of the primary metabolite Compound II are measured at pre-dose and at 0.5, 1, 1.5, 2, 3, 4, 6, 8, 12, and 24 hours post-dose. For all subsequent doses except the final dose, Plasma Compound I and Compound II concentrations are measured at trough, prior to the next dose. For the final dose on Day 7, plasma Compound I and Compound II concentrations are measured pre-dose and at 0.5, 1, 1.5, 2, 2.5, 3, 3.5, 4, 6, 8, 12, 16, 24, 36, and 48 hours post-dose.

### Food Effect:

Within the single dose portion of the study, the third cohort of subjects (A3) completes a second treatment period in which plasma Compound I and Compound II concentrations are measured at pre-dose and 0.5, 1, 1.5, 2, 2.5, 3, 3.5, 4, 6, 8, 12, 16, 24, 36, and 48 hours post-dose, following receipt of a high fat meal 30 minutes prior to dosing. PD assessments will note be performed for the Food Effect cohort.

Pharmacodynamic (PD) responses to be assessed are pupillometry, capnography, oximetry, and cold pressor testing. The order should be pupillometry, capnography, oximetry, and then cold pressor.

### Single Dose PD:

Pupillometry, capnography, and oximetry assessments occur pre-dose (Day −1) and at hours 3, 6, and 9 post-dose. For cold pressor, assessments occur at Screening (to confirm subject ability to perform the test), and then pre-dose (Day −1) and then at 4.5 and 8.5 hours post-dose.

### Multiple Dose PD:

Pupillometry, capnography, and oximetry assessments occur pre-dose and 3, 6, and 9 hours post-dose on Days 2, 4, and 6. For cold pressor, assessments occur at Screening (to confirm subject ability to perform the test), and then pre-dose (Day −1) and then at 3 hours post-dose on Days 1, 3, 5, and 7.

### Safety

The safety variables to be assessed include adverse events; clinical laboratory parameters (chemistry, hematology, and urinalysis; details in Table 5); 12-lead electrocardiograms (ECGs); physical examinations; and vital signs (including blood pressure, heart rate, oral temperature, and respiratory rate).

### Pharmacokinetics

The pharmacokinetic parameters to be calculated and analyzed are presented in the statistical methods.

### Statistical Methods: Sample Size

This is a first-in-human study, in which the single ascending dose and multiple ascending dose effects on tolerability, safety, and PK of Compound I are assessed. A sample size of eight subjects per cohort, 6 treated with Compound I and 2 treated with matching placebo, is used. The sample size allows for a careful escalation from lower to higher doses and adequately characterizes the rate and extent of drug absorption as measured by selected PK parameters.

### GENERAL CONSIDERATIONS

The analysis is structured consistent with the study objectives. Specifically, this study has two ‘Parts’, each with objectives that, as a whole, are intended to lead towards design of a future larger efficacy study in the target population.

Part A assesses safety and tolerability in a single-dose fashion in a multi-cohort dose-escalation design and includes a food effect assessment. Part B is a multiple ascending dose assessment. Both Parts are performed in healthy subject volunteers. Dosing in Part A begins at 20 mg and escalates through planned doses of 60 mg. 120 mg, 200 mg, and 400 mg: dosages may be adjusted based on findings in previous cohorts. Part B dosing begins after the 3^{rd} Part A cohort (Cohort A3, the fasting/fed cohort) has been completed; see Table 5 and Table 6 for the planned doses.

The starting dose of 20 mg in Part A is more than tenfold below the no-observed-adverse-effect level (NOAEL) in the most sensitive toxicology species. The chosen dose can be higher, as long as within the pharmacological range. The following NOAEL for dogs and rats are:
Dog: NOAEL=60 mg/kg/day/1.8 (km for dog)=33.3 mg/kg (HED)*50 kg subject=1665 mg
Rat: NOAEL=60 mg/kg/day/6.2 (km for rat)=9.7 mg/kg (HED)*50 kg subject=485 mg.

Part A is an ascending single-dose assessment of Compound I. Five cohorts 20 mg, 60 mg, 120 mg, 200 mg, 400 mg) are sequentially enrolled, with 6 active and 2 placebo subjects in each. Data are reviewed by the SRC to determine if the maximum tolerated dose (MTD) has been reached at each successive dosing level. The final statistical analysis allows for inspection of dose-related trends in safety, tolerability, and PK across the dose range studied. All subjects in Part A Cohorts A1, A2. A4, and A5 are dosed in a “fasted” condition. i.e., study treatment will be taken without food.

Cohort A3 of Part A determines the effect of a high fat meal on the pharmacokinetics of Compound I, and thus the focus is on comparison of pharmacokinetic outcomes between the fasted and fed condition of subjects treated with Compound I. Subjects who enroll in Cohort A3 are administered a single dose of their assigned treatment under fasted conditions in Period 1, and under fed conditions in Period 2. The sequence of the dosing is not randomized in this study, to allow for dosing in the fasted condition first.

Part B is an ascending multiple-dose (7 days) assessment of Compound I. Four cohorts (starting at 60 mg dose, with planned escalations to 100 mg, 200 mg and 400 mg; if 200 mg is not very well-tolerated a 300 mg dose may be substituted) are sequentially enrolled, with 6 active and 2 placebo subjects in each. Similar review by the SRC and final data presentations is provided to allow for dose-related trends across the dose range studied. All subjects in Part B are dosed in a fasted condition, i.e., study treatment is taken without food.

Consistent with the study design, the data summaries and statistical analyses are performed separately for each of Part A and Part B. Placebo-treated subjects are included in the safety analysis but are not included in the comparison of PK parameters.

### Pharmacokinetics

The actual blood sampling dates and times relative to dosing time are listed by subject and nominal sampling time, with time deviation calculated, for all subjects with available plasma concentration data. Individual (for each subject) and mean Compound I and metabolite Compound II concentrations over time (for each cohort) are displayed graphically in linear and semi-logarithmic plots. The actual collection time is used for individual plasma Compound I and Compound II concentration curves and the nominal time is used for the plots of mean plasma Compound I and Compound II concentration curves.

For PK concentration data, the number of non-missing values, number of below limit of quantification (BLQ) values, arithmetic mean, standard deviation, median, minimum, maximum, coefficient of variation (CV %), geometric mean and geometric coefficient of variation (geo CV %) values are presented. For the calculation of summary statistics, unrounded data are used and reported to three significant figures with the exception of n, n BLQ, and CV % which are presented to the nearest integer and one decimal place, respectively.

For PK parameter data, the number of non-missing values, arithmetic mean, standard deviation, median, minimum, and maximum values are presented. Individual PK parameters are presented to three significant figures with the exception of Tₘₐₓ, which are presented to two decimal places.

### Pharmacodynamics

Pharmacodynamic endpoints include pupillometry, capnography, oximetry, and the cold pressor test. Analyses focuses on the changes from pre-treatment (screening) to on-treatment time points and an assessment of the dose-response profiles of those changes.

### Assessment of Food Effect (Part A Cohort A3 Only)

For Part A Cohort A3, assessment of the food-effect is performed for the following PK parameters:
AUC₀₋ₗₐₛₜ
Cₘₐₓ
AUC_{0-inf}

Comparisons between treatment conditions (fed vs fasted) are evaluated by an analysis of the pharmacokinetic parameters by performing a fixed effect analysis of covariance (ANCOVA), with terms for subject and treatment (fed vs fasted) effects, on log-transformed values of Cₘₐₓ, AUC0-last and AUC0-inf Because all subjects have the same sequence (fed to fasted), there is no subject-within-sequence (random effect) in the model.

From these analyses, least-squares (LS) means, LS treatment differences, and 90% confidence interval (CI) for the treatment differences on log-scale are obtained for Cₘₐₓ, AUC₀₋ₗₐₛₜ and AUC_{0-inf}. The reference treatment i fasted condition for all comparisons. The results are transformed back to the original scale by exponentiation to provide treatment geometric LS means, point estimates of the LS mean ratios (test/reference, i.e., T/R), and 90% CI for these ratios and the least squared means.

Without wishing to be limited by any theory, in certain embodiments, due to the small sample size of this study, it can be concluded that the rate and extent of absorption is no different between the test and reference groups if the point estimate for AUC_{0-inf} (or AUC₀₋ₗₐₛₜ, if AUC_{0-inf} could not be reliably estimated for each fed/fasted condition in all subjects) and the Cₘₐₓ ratios fall within the range of 80 to 125%. Inter-subject and intra-subject coefficients of variation (CV %) are estimated and reported. PD assessments will not be performed for the food effect Cohort A3.

### Assessment of Steady State (Part B Only)

Using Part B data, determinations of steady state concentrations of Compound I and Compound II are performed by applying Helmert Contrasts to the trough (i.e., morning pre-dose) concentrations on Days 2, 3, 4, 5, 6, 7, and 8 within a mixed model repeated measure analysis of variance (ANOVA) model. The ANOVA model includes individual C_{trough} levels as the outcome (Y) variable, time point (Day) as the independent variable defined as a class variable and subject as a repeated measures effect to take into account the within subject correlation between the daily trough levels.

Each Helmert contrast compares the mean at a given time point to the pooled mean over all subsequent time points. The earliest time point for which the Helmert contrast is not statistically significant is considered to correspond to the dosing interval at which steady state is attained.

Within Helmert contrasts, the first contrast tested compares the mean concentration at the first time point (Day 2) to the pooled mean over all remaining time points (Days 3 to 8). The second contrast compares the mean at the second time point (Day 2) to the pooled mean over all remaining time points (Days 4 to 8). Testing continues until the contrast is not statistically significant at the p-value <0.05 level. The first time point included in this last contrast is concluded to be the dosing interval on which steady state is attained.

### Pharmacokinetic Parameters for Each Part

The following PK parameters are derived for Part A (Table 1) and Part B (Table 2), for both Compound I and Compound II. The definitions may be modified to accommodate the actual data collection and dosing intervals.

TABLE 1
Pharmacokinetic Parameters for Part A (Single Dose)

| Parameter | Definition |
|---|---|
| Cₘₐₓ | Maximum concentration which is directly determined from the plasma |
| | concentration time profiles |
| Tₘₐₓ | Time to maximum concentration. If the same Cₘₐₓ concentration occurs |
| | at different time points, Tₘₐₓ is assigned to the first occurrence of Cₘₐₓ |
| T_{lag} | Time of observation prior to the first observation with a measurable |
| | (non-zero) concentration (only applicable for the first dosing interval) |
| C₂₄ | The concentration observed at 24 hours post-dose |
| AUC₀₋ₜ | Area under the drug concentration-time curve, from time zero to the last |
| | measurable concentration using the ‘Linear Up and Log Down’ method |
| AUC₀₋ₗₐₛₜ | Area under the drug concentration-time curve, from time zero hour to 24 |
| | hours post-dose using the ‘Linear Up and Log Down’ method |
| AUC_{0-inf} | Area under the drug concentration-time curve, from time zero to infinity |
| | (∞) using the following formula |
| | ${AUC}_{0 - ∞} = {AUC}_{0 - t} + \frac{{C}_{t}^{′}}{{λ}_{z}}$ |
| | Where, C'ₜ is the observed concentration at the time t (last time point |
| | with a measurable plasma concentration above the quantification limit) |
| | at which quantification was still possible, the calculation of λ_{z} or kₑₗ is given below. |
| AUC_{%extrap} | The percentage of the AUC that has been extrapolated beyond the last |
| | observed data point, using the following formula |
| | ${AUC}_{\% extrap} = ( \frac{{AUC}_{0 - ∞} - {AUC}_{0 - t}}{{AUC}_{0 - ∞}} ) * 100$ |
| λ_{z} or kₑₗ | The apparent terminal elimination rate constant, will be estimated from a |
| | regression of In(C) versus time over the terminal log-linear drug |
| | disposition portion of the concentration-time profiles. |
| t_{1/2} | Apparent terminal half-life, using the following formula |
| | ${T}_{1 / 2} = \frac{Ln ( 2 )}{{λ}_{z}} ,$ |
| CL/F | Apparent total plasma clearance, using the following formula |
| | $CL / F = \frac{D}{{AUC}_{0 - ∞}}$ |
| | Where D = Administered dose; AUC_{0-∞} = AUC_{0-inf} |
| V_{z}/F | Apparent terminal volume of distribution, using the following formula |
| | ${V}_{z} / F = \frac{CL / F}{{λ}_{z}} Or \frac{Dose}{{λ}_{z} \times {AUC}_{inf}}$ |
| MRT | Mean residence time, using the following formula |
| | $MRT = \frac{{AUMC}_{0 - ∞}}{{AUC}_{0 - ∞}}$ |
| | AUMC_{0-∞} is the first moment of AUC_{0-∞}. |
| Cₘₐₓ/D | Dose-normalized Cₘₐₓ, calculated as Cₘₐₓ divided by the dose administered |
| AUC_{0-inf}/D | Dose-normalized AUC_{0-inf}, calculated as AUC_{0-inf} divided by dose administered |
| AUC₀₋ₜ/D | Dose-normalized AUC₀₋ₜ, calculated as AUC₀₋ₜ divided by dose administered |
| AUC₀₋ₗₐₛₜ/D | Dose-normalized AUC₀₋ₗₐₛₜ, calculated as AUC₀₋ₗₐₛₜ divided by the total |
| | dose administered in 24 hr. |

TABLE 2
Pharmacokinetic Parameters for Part B (Multiple Dose)

| Parameter | Definition |
|---|---|
| Cₘₐₓ | Maximum steady state concentration time over the dosing interval |
| Cₘᵢₙ | Minimum steady state concentration time over the dosing interval |
| C_{avg} | Average steady state concentration, using the following formula |
| | C_{avg} = AUC_{0-τ}/τ |
| | Where, τ = dosing interval |
| Tₘₐₓ | Time to reach maximum concentration. If the same Cₘₐₓ concentration |
| | occurs at different time points, Tₘₐₓ is assigned to the first occurrence of Cₘₐₓ. |
| Tₘᵢₙ | Time of minimum observed concentration |
| T_{lag} | Time of observation prior to the first observation with a measurable |
| | (non-zero) concentration (only applicable for the first dosing interval) |
| C₂₄ | The concentration observed at 24 hours post-dose |
| AUC₀₋ₜ | Area under the drug concentration-time curve, from time zero to the last |
| | measurable concentration using the ‘Linear Up and Log Down’ method |
| AUC_{0-τ} | Area under the drug concentration-time curve, from time zero hr to end |
| | of the dosing interval (τ) |
| AUC_{0-inf} | Area under the drug concentration-time curve, from time zero to infinity |
| | (∞) using the following formula |
| | ${AUC}_{0 - ∞} = {AUC}_{0 - t} + \frac{{C}_{t}^{′}}{{λ}_{z}}$ |
| | Where, C'ₜ is the observed concentration at the time t (last time point |
| | with a measurable plasma concentration above the quantification limit) at |
| | which quantification was still possible, the calculation of lambda_{z} or kₑₗ is given |
| | below. Will be determined after the first dose of the MAD regimen only. |
| AUC_{%extrap} | The percentage of the AUC that has been extrapolated beyond the last |
| | observed data point, using the following formula |
| | ${AUC}_{\% extrap} = ( \frac{{AUC}_{0 - ∞} - {AUC}_{0 - t}}{{AUC}_{0 - ∞}} ) * 100$ |
| λ_{z} or kₑₗ | The apparent terminal elimination rate constant, will be estimated from a regression |
| | of In(C) versus time over the terminal log-linear drug disposition portion of the |
| | concentration-time profiles. |
| t_{1/2} | Apparent terminal half-life, using the following formula |
| | ${T}_{1 / 2} = \frac{Ln ( 2 )}{{λ}_{z}} ,$ |
| CL/Fₛₛ | Apparent total plasma clearance, using the following formula |
| | $CL / {F}_{ss} = Dose / {AUC}_{0 - τ}$ |
| V_{z}/Fₛₛ | Apparent terminal volume of distribution, using the following formula |
| | $CL / {F}_{ss} = ( Dose ) / ( {lambda}_{z} \cdot {AUC}_{0 - τ} )$ |
| MRT | Mean residence time, using the following formula |
| | $MRT = \frac{AUC 0 - τ + τ ( {AUMC}_{0 - ∞} - AUC 0 - τ )}{AUC 0 - τ}$ |
| | AUMC_{0-∞} is the first moment of AUC. |
| Swing | Determined using the following formula |
| | $Swing = ( {C}_{max} - {C}_{min} ) / ( {C}_{min} )$ |
| Fluctuation% | Determined using the following formula |
| | $Fluctuation \% = 100 \cdot ( {C}_{max} - {C}_{min} ) / ( {C}_{avg} )$ |
| Cₘₐₓ/D | Dose-normalized Cₘₐₓ, calculated as Cₘₐₓ divided by the dose administered |
| AUC_{0-inf}/D | Dose-normalized AUC_{0-inf}, calculated as AUC_{0-inf} divided by dose administered |
| AUC₀₋ₜ/D | Dose-normalized AUC₀₋ₜ, calculated as AUC₀₋ₜ divided by dose administered |
| RA_{AUC} | Accumulation ratio for AUC = AUC₀₋ₜ on Day 7/AUC₀₋ₜ on Day 1 after the first dose |
| RA_{Cmax} | Accumulation ratio for Cₘₐₓ = Cₘₐₓ on Day 7/Cₘₐₓ on Day 1 after the first dose |

Note:
Following individual PK parameters, where possible, can be determined for the first dosing interval on Day 1 and the last on Day 7). Parameters on Day 7, if steady-state is attained, can be referred with the following nomenclature (as an example): Cmax,ss

### Pharmacokinetic Subgroups

While the sample sizes preclude formal comparison of PK parameters by subgroups, key PK parameters (specifically, Cₘₐₓ, AUC₀₋ₗₐₛₜ, AUC_{0-τ}, AUC_{0-inf}, Tₘₐₓ) are derived by sex (males vs females) for each dosing cohort. Group-mean concentration-time profiles by sex are provided (on the same plot, one for each dose within each Part) to allow for visual inspection of the overall exposure profiles.

### Safety

Safety data include adverse events, clinical laboratory, vital signs, ECGs, and physical examinations. Suicidality risk is assessed in the MAD part of the study.

TEAEs are defined as adverse events that occur following the first administration of study treatment. The onset time and date are collected for all TEAEs in Parts A and B. TEAEs is the focus of the analysis of adverse events.

All AEs are coded using MedDRA. All AE summaries include TEAEs only: AEs occurring prior to first dose of study treatment are listed but not included in any tabulations. Summary tables include the number of subjects (%) experiencing an event and the number of events. Subjects are counted only once for each system organ class (SOC) and preferred term (PT) level (categorical descriptive analysis).

The TEAE summaries include:
Overall summary of TEAEs
TEAE summary by SOC and PT
TEAE summary of serious events by SOC and PT
TEAE summary by SOC, PT and severity
TEAE summary by SOC, PT and relationship to study treatment (not related, possibly related or probably related)
TEAE summary of events leading to the study discontinuation by SOC and PT

Vital sign, clinical laboratory, and ECG measurements are presented using summary statistics for the results at baseline and each scheduled post-baseline visit for each of the parameters. In addition, summaries are presented for the change from baseline values at each scheduled post-baseline visit (continuous descriptive analysis).

### Study Schedule of Assessments

TABLE 3A
Schedule of Assessments - Part A (Single Dose)

| | | Confinement/Treatment Period | | | | Follow-up |
|---|---|---|---|---|---|---|
| | | (Period 1 and 2 for Cohort A3) Study Day | | | | Visit or Early |
| | Screening | Admission | | | | Termination |
| | Days −28 | to CRU | | | | Day 5 |
| Procedure | to −2 | Day −1 | Day 1 | Day 2 | Day 3 | (+2 days) |
| Informed consent | X | | | | | |
| Inclusion/Exclusion | X | X | | | | |
| Criteria | | | | | | |
| Demographics | X | | | | | |
| Medical and Social | X | X | | | | |
| History | | | | | | |
| Covid Test (antigen | X | X | | | | |
| or PCR test) | | | | | | |
| Height | X | | | | | |
| Weight | X | X | | | | X |
| Triplicate ECGs^{a} | X | X | X (t = −1, | X (24 h | X (48 hr | X |
| | | | 2, 4, 8 hr) | post-Day 1 | post-Day 1 | |
| | | | | dose) | dose) | |
| Vital signs | X | X | X (t = −1, | X (24 h | X | X |
| (respiration rate, | | | 1, 3, 6, 9, | post-Day 1 | | |
| oral temperature, | | | 12, 16 hr) | dose) | | |
| and orthostatic | | | | | | |
| [supine, sitting, | | | | | | |
| standing] blood | | | | | | |
| pressure and pulse)^{b} | | | | | | |
| Physical | | X (full) | | X | X | X |
| Examination^{c} | | | | (symptom) | (symptom) | (symptom) |
| Serology (HIV, | X^{d} | | | | | |
| HbsAg, and HCV) | | | | | | |
| Chemistry and | X | X | | X | X | X |
| Hematology^{e} | | | | | | |
| Urine drug/cotinine | X | X | | | | |
| Alcohol screen | X | X | | | | |
| (breath or urine) | | | | | | |
| Pregnancy (all | X | X | | | | X |
| females) ^{f} | | | | | | |
| FSH (females)^{g} | X | | | | | |
| Urinalysis | X | X | | X | X | |
| Randomization | | | X | | | |
| Treatment | | | X (t = 0) | | | |
| Administration | | | (30 minutes | | | |
| | | | after starting | | | |
| | | | a high fat | | | |
| | | | breakfast in | | | |
| | | | Period 2 for | | | |
| | | | Cohort A3) | | | |
| Sedation Assessment | | | X (~t = 2, | | | |
| | | | 4, 6 and | | | |
| | | | 8 hours) | | | |
| PK Samples ^{h} | | | X | X | X | |
| Prior/Concomitant | X | X | X | X | X | X |
| Medications | | | | | | |
| Adverse Eventsⁱ | | X^{e} | X | X | X | X |

Pharmacodynamic Assessments^{j}

| | | | |
|---|---|---|---|
| Pupillometry, | | X | X (~t = 3, |
| capnography, and | | | 6, 9 hours) |
| oximetry ^{k} | | | |
| Cold Pressor Test ^{k} | X | X | X (~t = 4.5, |
| | | | 8.5 hours) |

^{a}Triplicate ECGs are performed at Screening, Day −1, Day 1 [1 hour pre-dose, and 2, 4 and 8 hours post-dose], Day 2 [approximately 24 hours after Day 1 dose] and Day 3 [approximately 48 hours after the Day 1 dose], and during the follow-up visit. ECGs are collected prior to vital signs and blood collection whenever possible.
^{b}All three blood pressure assessments should be obtained at each timepoint, approximately 3 minutes apart.
^{c}A full physical exam is conducted on Day −1, while an abbreviated (symptom-driven) physical examination based on emergent changes occurs on all subsequent days and at safety follow up visit.
^{d}For subjects with a positive result for hepatitis C virus antibody, the blood sample is tested for hepatitis C virus RNA.
^{e}Following an a minimum 8-hour fast.
^{f} Females only, performed in serum at screening and in urine at all other times.
^{g}FSH completed in all females, regardless of menopausal or sterility status.
^{h} Pre-dose, 0.5, 1, 1.5, 2, 2.5, 3, 3.5, 4, 6, 8, 12, 16, 24, 36, and 48 hours post-dose (16 samples). All PK draws have a window of ±5 minutes from target draw time.
ⁱAny adverse events reported prior to dosing should be added to medical history and are not considered treatment-emergent adverse events.
^{j}Pupillometry is first, followed by capnography, then oximetry. Times are noted as approximate to allow for completion of the set of assessments.
^{k} Only for Cohorts A1-A2, A3 Period 1, and A4-5; do not perform pupillometry, oximetry, capnography or cold pressor test for Cohort A3 (Fed State in Food Effect cohort).

TABLE 3B
Schedule of Assements - Part B (Multiple Dose)

| | Study Phase | | | | | | | | | | | |
|---|---|---|---|---|---|---|---|---|---|---|---|---|
| | | | | | | | | | | | | Follow- |
| | | | | | | | | | | | | up Visit |
| | | | | | | | | | | | | or Early |
| | Screening | Cosfinement Study Period | | | | | | | | | | Termination |
| | Study Day(s) | | | | | | | | | | | |
| | | Admission | | | | | | | | | | |
| | −28 | to | | | | | | | | | | |
| | to −2 | CRU −1 | 1 | 2 | 3 | 4 | 5 | 6 | 7 | 8 | 9 | 15 (±2) |
| Informed Consent | X | | | | | | | | | | | |
| Inclusion/Exclusion | X | X | | | | | | | | | | |
| Criteria | | | | | | | | | | | | |
| Demographics | X | | | | | | | | | | | |
| Medical and Social | X | X | | | | | | | | | | |
| History | | | | | | | | | | | | |
| Covid test (antigen | X | X | | | | | | | | | | |
| or PCR) | | | | | | | | | | | | |
| Height | X | | | | | | | | | | | |
| Weight | X | X | | | | | | | | | | X |
| Single ECG^{a} | X | X | X | X | X | | X | | X | | X | X |
| Vital Signs/Oral | X | X | X (pre- | X (Pre-dose, 1 hr) | | | | | X (pre- | X (24, | X | X |
| Temp^{b} | | | dose, | | | | | | dose, | 32 hr) | | |
| | | | 1, 4, 8, | | | | | | 1, 4, 8, | | | |
| | | | 12 hr) | | | | | | 12 hr) | | | |
| Physical Exam ^{c} | | X | | | | | | | | | X | X |
| | | | | | | | | | | | (symptom) | (symptom) |
| Serology^{d} | | | | | | | | | | | | |
| Hematology & | X | X | | X | | | X | | | X | | X |
| Chemistry ^{e} | | | | | | | | | | | | |
| Urine drug/cotinine | X | X | | | | | | | | | | |
| screen | | | | | | | | | | | | |
| Alcohol Screen | X | X | | | | | | | | | | |
| (breath or urine) | | | | | | | | | | | | |
| Pregnancy (females) ^{f} | X | X | | | | | | | | | | X |
| FSH (females) ^{g} | X | | | | | | | | | | | |
| Urinalysis | X | X | | X | | | X | | | X | | |
| C-SSRS | X | | | | | | | | | X | | X |
| Randomization | | | X | | | | | | | | | |
| Treatment | | | X | X | X | X | X | X | X | | | |
| Administration ^{h} | | | X | X | X | X | X | X | X | | | |
| Sedation | | | (~t = | (~t = | (~t = | (~t = | (~t = | (~t = | (~t = | | | |
| Assessment | | | 2, 4, 6 | 2, 4, 6 | 2, 4, 6 | 2, 4, 6 | 2, 4, 6 | 2, 4, 6 | 2, 4, 6 | | | |
| | | | and 8 | and 8 | and 8 | and 8 | and 8 | and 8 | and 8 | | | |
| | | | hours) | hours) | hours) | hours) | hours) | hours) | hours) | | | |
| PK Samples ⁱ | | | X | X | X | X | X | X | X | X | X | X |
| Prior and Concomitant | X | X | X | X | X | X | X | X | X | X | X | X |
| Medications | | | | | | | | | | | | |
| Adverse Events | X^{j} | X^{j} | X | X | X | X | X | X | X | X | X | X |
| Pupillometry, | | X (single | | X | | X | | X | | | | |
| capnography, and | | timepoint | | (~t = | | (~t = | | (~t = | | | | |
| oximetry^{k, l} | | only) | | 3, 6, | | 3, 6, | | 3 , 6, | | | | |
| | | | | 9 hr) | | 9 hr) | | 9 hr) | | | | |
| Cold pressor test^{l, m} | X | X (single | X (~t- | | X (~t- | | X (~t- | | X (~t- | | | |
| | | timepoint | 3 hr) | | 3 hr) | | 3 hr) | | 3 hr) | | | |
| | | only) | | | | | | | | | | |

ECG = electrocardiogram, FSH = folicle stimulating hormone; PK = pharmacokinetic, C-SSRS = Columbia Suicide Severity Rating Scale
^{a}Single ECGs collected at pre-dose, and then hours 1 and 4 post-dose. ECGs will be collected prior to vital signs and blood collection whenever possible.
^{b}Vital signs including, orthostatic (supine, sitting, and standing) blood pressure, pulse, respiration rate and oral temperature will be collected at Screening, Day −1 (time matched to Day 1 pre-dose), Days 1-7 (pre-dose, and 1, 2, 4, and 8 hours post-dose), Day 8 (24 and 32 hours following Day 7 dose) and Day 9 (48 hours following Day 7 dose), and at follow up visit on Day 15.
^{c} A full physical examination will be conducted on Day −1 and symptom-driven physical exams will be conducted on Day 9 and at the follow-up visit.
^{d}Serology for HIV, HbsAg, and HCV.
^{e} Following an a minimum 8-hour fast
^{f} Pregnancy test completed in all females regardless of child-bearing potential. Serum-based testing at screening, all other tests are urine-based.
^{g} FSH completed in all females, regardless of menopausal or sterility status
^{h} Morning doses of Compound I or placebo will be administered after completing an overnight fast of at least 8 hours. Standard breakfast will be available approximately 90 minutes following morning dosing. Evening doses, if included (see text above), will be administered 12 hours after the morning dose and at least 1 hour after completing the evening meal. However, dosing and food considerations (fed or fasted) for the MAD portion of the study may change pending outcomes from the SAD and food-effect assessments.
ⁱ Blood samples for Compound I concentrations will be drawn on Day 1 and Day 7 at pre-dose, 0.5, 1, 1.5, 2, 2.5, 3, 3.5, 4, 6, 8, 12, and 16 hours post-dose. A pre-dose trough sample will be collected on Days 2-6. Additional samples at 24 hours and 36 hours post-dose will be collected on Day 8, and a sample 48 hours following the last dose will be drawn on the morning of Day 9. A final blood draw will be made at the follow up visit, all PK blood draws have a window of ±5 minutes from the target draw time.
^{j}Any adverse events reported prior to dosing should be added to medical history and are not considered treatment-emergent adverse events.
^{k}Site can stagger times to accommodate, but the order should be pupillometry, capnography, oximetry, and then cold pressor.
^{l}Pupillometry, oximetry, and capnography assessments to be obtained at 3, 6, and 9 hours post-dose on Days 2, 4, and 6, and at the equivalent of 3 hours post-dose on Day −1, if possible.
^{m}Cold pressor test to be performed at Screening, at the equivalent of 3 hours post-dose on Day −1 and at 3 hours post-dose on Days 1, 3, 5. and 7.

### SSRS=Columbia Suicide Severity Rating Scale

a. Single ECGs collected at pre-dose, and then hours 1 and 4 post-dose. ECGs will be collected prior to vital signs and blood collection whenever possible.
b. Vital signs including, orthostatic (supine, sitting, and standing) blood pressure, pulse, respiration rate and oral temperature will be collected at Screening, Day −1 (time matched to Day 1 pre-dose), Days 1-7 (pre-dose, and 1, 2, 4, and 8 hours post-dose), Day 8 (24 and 32 hours following Day 7 dose) and Day 9 (48 hours following Day 7 dose), and at follow up visit on Day 15.
c. A full physical examination will be conducted on Day −1 and symptom-driven physical exams will be conducted on Day 9 and at the follow-up visit.
d. Serology for HIV, HbsAg, and HCV.
e. Following an a minimum 8-hour fast
f. Pregnancy test completed in all females regardless of child-bearing potential. Serum-based testing at screening, all other tests are urine-based.
g. FSH completed in all females, regardless of menopausal or sterility status
h. Morning doses of Compound I or placebo will be administered after completing an overnight fast of at least 8 hours. Standard breakfast will be available approximately 90 minutes following morning dosing. Evening doses, if included (see text above), will be administered 12 hours after the morning dose and at least 1 hour after completing the evening meal. However, dosing and food considerations (fed or fasted) for the MAD portion of the study may change pending outcomes from the SAD and food-effect assessments.
i. Blood samples for Compound I concentrations will be drawn on Day 1 and Day 7 at pre-dose, 0.5, 1, 1.5, 2, 2.5, 3, 3.5, 4, 6, 8, 12, and 16 hours post-dose. A pre-dose trough sample will be collected on Days 2-6. Additional samples at 24 hours and 36 hours post-dose will be collected on Day 8, and a sample 48 hours following the last dose will be drawn on the morning of Day 9. A final blood draw will be made at the follow up visit, all PK blood draws have a window of ±5 minutes from the target draw time.
j. Any adverse events reported prior to dosing should be added to medical history and are not considered treatment-emergent adverse events.
k. Site can stagger times to accommodate, but the order should be pupillometry, capnography, oximetry, and then cold pressor.
l. Pupillometry, oximetry, and capnography assessments to be obtained at 3, 6, and 9 hours post-dose on Days 2, 4, and 6, and at the equivalent of 3 hours post-dose on Day −1, if possible.
m. Cold pressor test to be performed at Screening, at the equivalent of 3 hours post-dose on Day −1 and at 3 hours post-dose on Days 1, 3, 5, and 7.

### LIST OF ABBREVIATIONS AND DEFINITIONS OF TERMS

The following abbreviations and specialist terms are used in this study protocol.

TABLE 4
Abbreviations and Specialist Terms

| Abbreviation or | |
|---|---|
| Specialist Term | Explanation |
| % AUCₑₓₜ | Percentage of AUC_{0-inf} extrapolated from the last data |
| | point to infinity |
| AE | Adverse Event |
| ALB | Albumin |
| ALP | Alkaline Phosphatase |
| ALT | Alanine aminotransferase |
| AMP | Amphetamines |
| APTT | Activated partial thromboplastin time |
| AST | Aspartate aminotransferase |
| ATC | Anatomical therapeutic chemical |
| AUC_{0-τ} | Area under the drug concentration-time curve, from time |
| | zero hr to end of the dosing interval (τ) |
| AUC₀₋₁₂ | Area under the plasma concentration curve from time |
| | zero to 12 h |
| AUC_{0-inf} | Area under the plasma concentration curve from time |
| | zerot o infinity |
| AUC₀₋ₗₐₛₜ | Area under the plasma concentration curve from time |
| | zero to time of last data point |
| AUC₀₋ₗₐₛₜ/D | Dose-normalised AUC₀₋ₗₐₛₜ |
| BAR | Barbiturates |
| BASO | Basophils |
| BICARB | Bicarbonate |
| BID | Twice daily |
| BILI | Total Bilirubin |
| BILIDIR | Direct Bilirubin |
| BIS | Bioelectrical impedance spectroscopy |
| BMI | Body Mass Index |
| BP | Blood pressure |
| bpm | Beats per minute |
| BZO | Benzodiazepines |
| C₂₄ | Concentration at 24 h |
| CA | Calcium |
| C_{avg} | Mean plasma concentration |
| cGMP | Current good manufacturing practice |
| CI | Confidence interval |
| CL | Chloride |
| CL/F | Apparent total clearance |
| CLss/F | Apparent clearance at steady state |
| Cₘₐₓ | Observed maximum plasma concentration |
| Cₘₐₓ/D | Dose-normalised Cₘₐₓ |
| COC | Cocaine |
| CPK | Creatine phosphokinase |
| CREAT | Creatinine |
| CRO | Clinical Research Organisation |
| CRP | C-reactive protein |
| CRU | Clinical research unit |
| CSR | Clinical study report |
| C_{trough} | Trough concentration |
| C_{trough}/D | Dose-normalised trough concentration |
| ECG | Electrocardiogram |
| eCRF | Electronic case Report Form |
| ESN | Eosinophils |
| FGF-2 | Fibroblast Growth Factor-2 |
| FNA | Fine needle aspiration |
| FSH | Follicle-stimulating hormone |
| GCP | Good Clinical Practice |
| G-CSF | Granulocyte colony stimulating factor |
| GGT | Gamma glutamyl transpeptidase |
| GLOBUL | Globulin |
| GLU | Glucose |
| HBsAg | Hepatitis B surface antigen |
| HCT | Haematocrit |
| HCV | Hepatitis C virus |
| HGB | Haemoglobin |
| HIV | Human immunodeficiency virus |
| HR | Heart rate |
| HREC | Human Research Ethics Committee |
| IB | Investigators Brochure |
| ICF | Informed Consent Form |
| ICH | International Council for Harmonisation |
| IL-10 | Interleukin 10 |
| IL-1β | Interleukin 1 beta |
| IL-4 | Interleukin 4 |
| IL-6 | Interleukin 6 |
| INR | International normalised ratio |
| IP | Investigational product |
| IPF | idiopathic pulmonary fibrosis |
| ITT | Intent-to-treat |
| IUD | Intrauterine device |
| IV | Intravenous |
| K | Potassium |
| lambda z | Terminal elimination rate constant |
| LFT | Liver function test |
| LYM | Lymphocytes |
| LYMQOL | Lymphoedema Quality of Life Tool, Arm |
| MAD | Multiple ascending dose |
| MCP-1 | Monocyte Chemoattractant Protein-1 |
| MDMA | Methylenedioxymefvthamphetamine |
| MedDRA ® | Medical Dictionary for Regulatory Activities |
| MM | Medical Monitor |
| MRT | Mean residence time |
| MTD | Methadone |
| MTD | Maximum tolerated dose |
| n | Number |
| NA | Sodium |
| NEUT | Neutrophils |
| NSAID | Nonsteroidal anti-inflammatory drug |
| OAAS | Observer's Assessment of Alertness and Sedation |
| OPI | Opiates |
| OTC | Over the counter |
| OUD | Opiate Use Disorder |
| PCP | Phencyclidine |
| PD | Pharmacodynamics |
| PE | Physical examination |
| PHOS | Phosphate |
| PI | Principal Investigator |
| PK | Pharmacokinetics |
| PLAT | Platelets |
| PP | Pharmacokinetic population |
| PROT | Protein |
| PT | Preferred term |
| PTF | Peak trough fluctuation |
| QoL | Quality of life |
| QTcF | QT interval corrected using Fridericia's formula |
| Rac(AUC) | Accumulation ratio calculated from the area under the |
| | plasma concentration time curve |
| RBC | Red blood cells |
| RETI | Reticulocytes |
| RPSD | Randomization plan and specifications document |
| RR | Respiration rate |
| SAE | Serious Adverse Event |
| SAP | Statistical Analysis Plan |
| SD | Standard deviation |
| SOC | System organ class |
| SPGRAV | Specific Gravity |
| SRC | Safety Review Committee |
| t_{1/2} | Elimination half-life |
| TDC | Tissue dielectric constant |
| TEAE | Treatment-emergent adverse event |
| THC | Tetrahydrocannabinol |
| TID | Three times a day |
| TIMP-1 | Tissue inhibitor of matrix metalloproteinase 1 |
| t_{lag} | Lag time |
| Tₘₐₓ | Time of observed maximum plasma concentration |
| U | Urea |
| UROBIL | Urobilinogen |
| Vz/F | Apparent volume of distribution during the terminal |
| | phase |
| Vzss/F | Apparent volume of distribution at steady state |
| WBC | Leukocytes |
| WHO | World Health Organisation |
| WOCBP | Woman of childbearing potential |

TABLE 5
Part A Planned Dose Levels - Single
Doses (Study Compound I-101)

| | | Total | | |
|---|---|---|---|---|
| | Planned | Amount (mg) | Number of Subjects | |
| | Dose of | Compound I | | Compound |
| Cohort | Compound I | (or Placebo) | Placebo | I |
| A1 | 20 mg Fasting | 20 mg | 2 | 6 |
| A2 | 60 mg Fasting | 60 mg | 2 | 6 |
| A3 | Period 1: | 240 mg | 2 | 6 |
| (2-period FE) | 120 mg Fasting | | | |
| | Period 2: | | | |
| | 120 mg Fed | | | |
| A4 | 200 mg Fasting | 200 mg | 2 | 6 |
| A5 | 400 mg Fasting | 400 mg | 2 | 6 |

FE = Food Effect
For all cohorts EXCEPT the food-effect cohort, morning doses will be administered after completing an overnight fast of at least 8 hours. Standard breakfast will be available approximately 90 minutes following morning dosing.
For the food-effect cohort, dosing will be administered in a fasted state and then in a fed state.
Note:
Further Cohorts may be added as the data evolve and/or dose levels adjusted based on outcomes in prior Cohorts.

### Part A:

All Part A cohorts are dosed with a single morning dose only, in a fasted state.

Additional cohorts and intermediate doses can be selected in lieu of predefined doses as noted and in accordance with safety and tolerability responses, but these do not exceed 400 mg for the SAD portion of the study.

If clinically significant safety signals assessed as >Mild/Grade 1 and thought to be possibly related to study treatment are observed in the 2 sentinel subjects in advance of dosing the remaining 6 subjects, the SRC reviews safety data before the remaining 6 subjects are treated.

Up to 40 subjects are enrolled in Part A (n=6 Compound I and n=2 placebo in each cohort), unless additional intermediate cohorts are needed. Subjects are admitted to the CRU on the evening of Day −1 and are discharged on Day 3 in the absence of clinically significant safety signals. A follow-up safety assessment is performed in the clinic on Day 5. Eligibility for Period 2 is reviewed prior to treatment, and an approximate 6-day washout is included between the two dosing days.

For the Cohort A3 fed period (Period 2), dosing is 30 minutes after the start of consumption of their standardized (high fat) breakfast.

For all subjects in all cohorts, standardized lunch and dinner are served. Subjects return to the study for a follow-up visit on Day 5 (+2 days) for a final safety assessment. For all cohorts in Part A, the decision to escalate or modify the dose prior to dosing of the next Cohort is determined by the SRC.

### Part B: Treatment Period

Part B comprises a multiple ascending dose (MAD), sequential cohort study. This part is initiated after the first three SAD cohorts have been fully evaluated for safety and tolerability and the safety review committee has concluded that the MAD portion may commence. Up to 32 subjects are studied in 4 cohorts (Cohorts B1 to B4), each cohort consisting of 8 subjects.

In each of Cohorts B1 to B4, 8 subjects receive Compound I and 2 receive placebo. Once daily dosing occurs on Days 1 to 7, inclusive, for all subjects. Each subject participates in 1 treatment period only, residing at the CRU from the evening of Day −1 (the day before first dose) until the morning of Day 9 (48 hours after the final dose, given on the morning of Day 7). All subjects return for a poststudy visit on Day 15 (±2 days) for a final safety assessment. Table 6 provides the Part B planned dose levels.

TABLE 6
Part B Planned Dose Levels - Multiple
Dosing (Study Compound I-101)

| | | | Total | | |
|---|---|---|---|---|---|
| | | | Dose | Number of Subjects | |
| | Planned Daily | Number | (mg) | | Com- |
| Co- | Dose of | of Days | Compound I | | pound |
| hort | Compound I | Dosed | (or Placebo) | Placebo | I |
| B1 | 60 mg Fasting | 7 | 420 mg | 2 | 6 |
| B2 | 100 mg Fasting | 7 | 700 mg | 2 | 6 |
| B3 | 200 mg Fasting | 7 | 1400 mg | 2 | 6 |
| B4 | 300 mg or 400 mg ^{a} | 7 | 2100 or 2800 | 2 | 6 |
| | Fasting | | mg | | |

Note:
Further Cohorts may be added as the data evolve.
^{a} If 200 mg dose is well-tolerated, dose has final escalation to 400 mg. If 400 mg dose is not well-tolerated, a 300 mg dose group may be tested.

For all cohorts, morning doses are administered after completing an overnight fast of at least 8 hours. Standard breakfast is available approximately 90 minutes following morning dosing. Evening doses are administered 12 hours after the morning dose and at least 1 hour after completing the evening meal. However, dosing and food considerations (fed or fasted) for the MAD portion of the study may change pending outcomes from the SAD and food-effect assessments.

### Number of Subjects

Part A includes 40 healthy subjects, while Part B includes 32 healthy subjects, and thus a total of 72 subjects are planned. Additional cohorts (and therefore additional subjects) may be added as the data evolve. Replacement of subjects who drop out is not planned unless fewer than 4 subjects complete within a given cohort either Part A or Part B of the study; however, enrolment of additional of subjects at any given dose level (cohort) or new cohort is possible as the data evolve.

### Safety Oversight and Stopping Rules

The study is overseen by a Safety Review Committee (SRC). The SRC convenes to review any safety signals deemed relevant relative to the study conduct i.e., incidence and nature of any AEs, serious adverse events (SAEs), vital sign changes, changes in physical findings, ECGs and laboratory abnormalities.

### Individual Subject Stopping Rules for Part A

As Part A uses a single dose treatment, no criteria are necessary to discontinue dosing for an individual subject in this Part, except that subjects in Cohort A3 who would have participated in the Food Effect Period 2 (Fed State) will not do so if they experience:
Any serious or severe (i.e., Grade 3) adverse event or laboratory abnormalities, or major ECG finding. Grade 3 toxicities are defined in Appendix 1 (FDA Guidance 2007). Major ECG findings include atrial fibrillation or flutter, high-degree atrioventricular dissociation, left bundle-branch block, right bundle-branch block, indeterminate conduction delay, isolated ischemic abnormalities, left ventricular hypertrophy with ST-T abnormalities, and other miscellaneous arrhythmias (e.g., supraventricular tachycardia, ventricular preexcitation, ventricular tachycardia (Denes 2007).
Any serious or severe (Grade 3, per Appendix 1) change in vital signs or physical examination as determined by the investigator.

### Individual Subject Stopping Rules for Part B

Individual subjects in Part B will be discontinued if they experience:
Any serious or severe (i.e., Grade 3) adverse event or laboratory abnormalities, or major ECG finding. Grade 3 toxicities are defined in Appendix 1 (FDA Guidance 2007). Major ECG findings include atrial fibrillation or flutter, high-degree atrioventricular dissociation, left bundle-branch block, right bundle-branch block, indeterminate conduction delay, isolated ischemic abnormalities, left ventricular hypertrophy with ST-T abnormalities, and other miscellaneous arrhythmias (e.g., supraventricular tachycardia, ventricular preexcitation, ventricular tachycardia. (Denes 2007)
Any serious or severe (Grade 3, per Appendix 1) change in vital signs or physical examination as determined by the investigator.

At any phase of the study, administration of study treatment to all subjects will be paused for dose-limiting toxicity as defined below. Dose-limiting toxicity is defined as follows:
Occurrence of two severe (i.e., Grade 3, per Appendix 1; FDA Guidance 2007) adverse events, laboratory abnormalities, vital sign readings or major ECG findings (specified in individual stopping rules; Denes 2007).
Any serious adverse event (SAE) that is considered at least possibly related to study treatment; or
An AE or group of AEs that singularly or in aggregate indicate that the study treatment is poorly tolerated and further treatment per protocol may not be safe.
Any serious or severe potentially opiate-related AEs (ORAEs), defined as bradypnea, constipation, dizziness, hypoxia, respiratory disorder, nausea, somnolence, sedation, vomiting, and pruritus.

### Subject Inclusion Criteria

To be eligible for this study, subjects must meet all of the following inclusion criteria:
1. Provides written IRB-approved informed consent prior to any study procedures.
2. Male or female between 18 and 55 years old (inclusive) at the time of screening.
3. In good general health at screening, free from clinically significant unstable medical, surgical or psychiatric illness, at the discretion of the Investigator.
4. Subjects have a BMI between ≥18.0 and ≤32.0 kg/m² at screening.
5. Vital signs (measured in supine position after a 5-minute rest) at screening:
   a. Systolic blood pressure ≥90 and ≤140 mmHg
   b. Diastolic blood pressure ≥50 and ≤90 mmHg
   c. Heart rate ≥45 and ≤100 bpm
   d. Temperature ≥35.5° C. and≤37.5° C.
   e. Vital signs may be repeated once, within a minimum of 10 minutes of the completion of the last set of vital signs (while maintaining supine position until the repeated set of vital signs are collected), if it is suspected that falsely high or low levels have been obtained.
   6. No clinically significant ECG or laboratory abnormalities. Specifically, liver function tests, hematocrit, and WBC must be within normal limits as defined by the clinical laboratory.
   7. Adequate venous access to allow collection of multiple blood samples.
   8. Negative Covid PCR test upon admission to the CRU.
      a. Subjects in Cohort A3 will need a second COVID test prior to admission the CRU for Period 2.
   9. No relevant dietary restrictions and willingness to consume standard meals and snacks.
   10. Willing to comply with all study procedures and requirements
   11. Ability to tolerate the cold pressor test (determined at screening)
   12. Women of childbearing potential (WOCBP) must be non-pregnant and non-lactating, and must use two acceptable, highly effective methods of contraception from screening until study completion, including the follow-up. Abstinence as a lifestyle choice is also acceptable. WOCBP must have a negative serum pregnancy test at Screening and negative urine pregnancy test at Day −1 and be willing to have additional pregnancy tests as required throughout the study. WOCBP must also use two acceptable, highly effective methods of contraception from screening until study completion, including the follow-up period and for 30 days after the last dose (please see Section 10.4.2 for acceptable methods of contraception). Women not of childbearing potential must be post-menopausal for ≥12 months or be surgically sterile. Hysterectomy with retention of ovary function is permitted. Post-menopausal status will be confirmed through testing of FSH levels ≥40 IU/mL at screening for amenorrhoeic female subjects.
   13. Male subjects must be surgically sterile (>30 days since vasectomy per medical history or verbal confirmation), or, if engaged in sexual relations with a WOCBP, the subject and his partner must use two acceptable, highly effective methods of contraception from screening until study completion, including the follow-up period and 30 days after the last dose. Abstinence as a lifestyle choice is also acceptable.

### Subject Exclusion Criteria

To be eligible for this study, subjects must not meet any of the following exclusion criteria:
1. Pregnant or lactating at screening or baseline or planning to become pregnant (self or partner) at any time during the study, including the specified follow-up period.
2. History or presence of malignancy at screening or baseline, with the exception of adequately treated localised skin cancer (basal cell or squamous cell carcinoma) or carcinoma in-situ of the cervix.
3. Clinically significant infection within 28 days of the start of dosing, or infections requiring parenteral antibiotics within the 6 months prior to screening.
4. Clinically significant surgical procedure within 3 months of screening, at the discretion of the Investigator.
5. Currently suffering from clinically significant systemic allergic disease at screening or baseline or has a history of significant drug allergies including a history of anaphylactic reaction; allergic reaction due to any drug which led to significant morbidity.
6. Chronic administration (defined as more than 14 consecutive days) of immunosuppressants or other immune-modifying drugs within 3 months prior to study treatment administration; corticosteroids are permitted at the discretion of the Investigator), or exposure to any significantly immune suppressing drug within 30 days prior to screening or 5 half-lives, whichever is longer.
7. History or presence at screening or baseline of a condition associated with significant immunosuppression.
8. Positive test for hepatitis C (HCV), hepatitis B (HBsAg), COVID, or human immunodeficiency virus (HIV) antibody at screening.
9. Symptoms of dysphagia at screening or baseline or known difficulty in swallowing capsules.
10. Any condition at screening or baseline (e.g., chronic diarrhea, inflammatory bowel disease or prior surgery of the gastrointestinal tract) that would interfere with drug absorption or any disease or condition that is likely to affect drug metabolism or excretion, at the discretion of the Investigator.
11. History or presence at screening or baseline of clinically significant cardiac arrhythmia or congenital long QT syndrome.
12. QT interval corrected using Fridericia's formula (QTcF)>450 msec for males or >470 msec for females.
13. Abnormal liver function tests, hematocrit, or WBC outside the normal limits as defined by the clinical laboratory
14. Clinically significant findings on the C-SSRS at screening, as determined by the investigator.
15. Use of tobacco or nicotine containing products in the previous month prior to dosing or a positive urine cotinine test at Screening or Baseline.
16. Lack of willingness to abstain from the consumption of tobacco or nicotine-containing products throughout the duration of the study and until completion of the final Follow-up visit.
17. Regular alcohol consumption defined as >21 alcohol units per week (where 1 unit=284 mL of beer, 25 mL of 40% spirit or a 125 mL glass of wine) or the subject is unwilling to abstain from alcohol for 48 h prior to admission and 48 h prior to Follow-up study visit.
18. Positive toxicology screening panel [urine test, including qualitative identification of barbiturates, tetrahydrocannabinol (THC), amphetamines, benzodiazepines, opiates and cocaine] or alcohol test (breath or urine) during Screening or at any time during the Study.
19. History of substance abuse or dependency or history of recreational IV drug use, over the last 5 years.
20. Use of any prescription drugs (other than permitted contraception) within 14 days prior to dosing or throughout the duration of the study, without prior approval of the Investigator and written approval of the CRO Medical Monitor.
21. Use of OTC medication including nonsteroidal anti-inflammatory drugs (NSAIDs), herbal remedies, supplements, or vitamins within 7 days prior to dosing. An exception to this is use of acetaminophen up to 4 g/day to treat mild discomfort.
22. Use of any investigational drug or device within 30 days or 5 half-lives, whichever is longer, prior to screening.
23. Clinically significant blood loss, or blood or blood product donation >250 mL within 28 days of screening.
24. Subject is unwilling to refrain from strenuous exercise from 7 days prior to admission to the clinical trial unit until completion of the final onsite follow-up visit, where strenuous exercise is defined as a significant increase in the Subject's usual level of physical activity.
25. Any other reason that, in the opinion of the Investigator, might interfere with the evaluation required by the study.

### Blood and Urine Sample Collection

Blood samples (for PK, clinical laboratories for safety) and urine samples (for screening and safety evaluations) are obtained at the time points delineated in the applicable Schedule of Assessments (see Table 2 and Table 3). The total volumes of blood to be taken from each subject in Parts A and B and are detailed in the Appendix 2 Table 1 and Appendix 2 Table 2, respectively.

### Pharmacokinetics

In Part A, blood samples (4 mL each) for pharmacokinetics are collected for Cohorts A1 through A5 at each time as follows:
Day 1 (pre-dose), 0.5, 1, 1.5, 2, 2.5, 3, 3.5, 4, 6, 8, 12, and 16 hours
Day 2, 24 hours
Day 3, 48 hours
For Cohort A3, the same PK collection as above is performed for Period 1 and for Period 2

In Part B, blood samples (4 mL each) for pharmacokinetics are collected as follows:
Day 1 (pre-dose), 0.5, 1, 1.5, 2, 2.5, 3, 3.5, 4, 6, 8, 12, and 16 hours
Day 2, 24 hours (immediately pre-dose)
Day 3, immediately pre-dose
Day 4, immediately pre-dose
Day 5, immediately pre-dose
Day 6, immediately pre-dose
Day 7, immediately pre-dose, 0.5, 1, 1.5, 2, 2.5, 3, 3.5, 4, 6, 8, 12, and 16 hours
Day 8: 24 hours and 36 hours post-Day 7 dose
Day 9: 48 hours post-Day 7 dose

### Sample Analysis

If the metabolite Compound II is found in the early SAD cohorts to have such low concentrations as to be unimportant to the description of the pharmacokinetics of Compound I, bioanalysis of plasma samples may not be performed for detection of Compound II in later SAD and/or MAD cohorts.

### Pharmacodynamic Endpoints

For Part A (single dose), pupillometry, capnography, and oximetry assessments are made on Day −1, and then at 3, 6, and 9 hours post-dose. Cold pressor testing is performed at Screening, Day −1, and then post-dose (Day 1) at 4.5 and 8.5 hours.

For Part B (multiple dose), these assessments are made on Day −1 and then on Days 2, 4, and 6, at hours ~t=3, 6, and 9 post-dose. Cold pressor testing is performed at Screening, on Day −1 (at the equivalent of 3 hours post-dose), and 3 hours post-dose on Days 1, 3, 5, and 7.

The site can stagger these times to accommodate daily schedule, but the order should be pupillometry, capnography, oximetry, and then cold pressor.

### Capnography

EtCO2 is measured in millimeters of mercury (mm Hg) using noninvasive capnography.

A Masimo/Philips EMMA® Capnograph (Americas Headquarters: Masimo Corporation, 52 Discovery, Irvine, CA 92618, USA) is used with an airway adapter and subject mouthpiece. The EMMA Capnograph provides clear, continuous capnograph of carbon dioxide values, is simple, easy-to-use, with audible and visual alarm system for No Adapter, Clogged Adapter, No Breath (Apnea), Low Battery and adjustable High and Low EtCO2 alarm. Subjects are instructed to breathe normally through the mouthpiece for 1 minute (according to the instructions provided with the capnograph).

### Oximetry

Oximetry measures oxygen saturation of circulating blood (SpO2). Measurements should be performed using the same finger during the course of the subjects' participation. For each time point indicated, a single SpO2 measure is recorded.

### Pupillometry

Pupil size and reactivity are measured using a pupillometer device such as the NeuroOptics NPi-300 pupillometer. All pupillometer measurements are made in the same windowless room, using standardized dim lighting.

### Cold Pressor Test

Subjects are asked to place their left hand and forearm into an apparatus containing an ice bath, with the instruction to remove the arm from the water when they can no longer tolerate it. The upper limit for the duration of the test, which is not be communicated to the subjects, is 5 minutes (300 seconds).

### Assessment of Safety

### Vital Signs

Vital signs (oral body temperature, blood pressure, heart rate, respiratory rate) are measured at the time points specified in the study schedules with subjects resting for at least 5 minutes in a supine position. When the time of vital signs measurement coincides with a blood draw, the vital signs are taken before the scheduled blood draw where possible while ensuring the blood draw is within the window specified in the protocol.

Additional vital signs may be performed at other times if deemed necessary.

### Weight and Height

Body height (centimetres) and body weight (kilograms) are measured at the time points delineated in the study schedules and are used to calculate BMI. BMI is calculated by dividing the subject's body weight in kilograms by the subject's height in meters squared (kg/m²). Body weight and height are obtained with the subject's shoes and jacket, or coat removed.

### Physical Examination

Complete physical examinations are performed by a licensed physician, nurse practitioner or physician's assistant at the time points specified in the study schedules.

Complete physical examinations include: general appearance, head, ears, eves, nose, throat, dentition, thyroid, chest (heart, lungs), abdomen, skin, extremities, back, neck, musculoskeletal, and lymph nodes.

Physical examinations are performed at various unscheduled time points if deemed necessary by the Investigator.

### Electrocardiogram

A 12-lead ECG is taken at the time points delineated in the study schedules.

Additional ECG monitoring may be performed at other times if deemed necessary. Note that triplicate ECGs are required in Part A, while single ECGs are required in Part B.

ECGs are performed prior to vital signs with subjects in a supine position. Subjects must be in this position for at least 5 minutes before the reading is taken.

All ECG tracings are reviewed by the PI or designee.

When the time of ECG monitoring coincides with a blood draw, the ECG is taken before the scheduled blood draw while ensuring the blood draw is within the window specified in the protocol.

### Laboratory Assessments

Safety laboratory tests (hematology, biochemistry, and urinalysis) are performed at the time points specified in the study schedules. Additional clinical laboratory tests may be performed at other times if deemed necessary based on the subject's clinical condition.

Medically indicated laboratory tests (emergency or unscheduled tests) should be conducted at the local laboratory.

A blood sample is taken from each subject for hematology and biochemistry analyses at the time points delineated in the study schedules.

### Hematology

Hematology parameters to be tested are:
Hemoglobin (HGB)
Hematocrit (HCT)
Erythrocytes (RBC)
Platelets (PLAT)
Leukocytes with differential (including Eosinophils (ESN), Neutrophils (NEUT), Basophils (BASO), Lymphocytes (LYM) and Reticulocytes (RETI)

### Biochemistry

Biochemistry parameters to be tested are:
C-reactive protein (CRP)
Urea (U)
Creatinine (CREAT)
Total Bilirubin (BILI)
Direct Bilirubin (BILIDIR)
Urate (URATE)
Albumin (ALB)
Alkaline Phosphatase (ALP)
Creatine phosphokinase (CPK)
Troponin 1 (TROPI)
Aspartate Aminotransferase (AST)
Alanine Aminotransferase (ALT)
Gamma—glutamyl transpeptidase (GGT)
Glucose (GLU) (fasting labs only)
Sodium (NA)
Potassium (K)
Calcium (CA)
Chloride (CL)
Phosphate (PHOS)
Bicarbonate (BICARB)

### Urinalysis

A urinalysis is performed for each subject. Urinary analysis is performed at Screening and other times according to the study schedule. If abnormality is noted for protein, blood, nitrite, or leukocyte esterase (and at the discretion of the Investigator) a microscopic examination of RBC, WBC, bacteria and casts is performed.

Macroscopic urinalysis parameters to be tested are:
pH (PH)
Specific Gravity (SPGRAV)
Creatinine (CREATININE)
Protein (PROT)
Glucose (GLUC)
Ketones (KETONES)
Total Bilirubin (BILl)
Occult Blood (OCCBLD)
Nitrite (NITRITE)
Urobilinogen (UROBIL)
Leukocytes (WBC)

### Viral Serology

HBsAg, anti-HCV, and HIV antibody testing are performed at Screening. COVID PCR test is performed at Screening and upon admission to the CRU.

### Urine Drug Screen

A urine drug screen is performed at Screening and at admission to the CRU (Day −1). This includes screening for:
Amphetamines (AMP)
Methamphetamines (MET)
Methadone (MTD)
Barbiturates (BAR)
Benzodiazepines (BZO)
Cocaine (COC)
Opiates (OPI)
Methylenedioxymethamphetamine (MDMA)
Phencyclidine (PCP)
THC

A urine cotinine test is performed at Screening and at admission to the CRU (Day −1).

### Adverse and Serious Adverse Events

Safety and tolerability are assessed throughout Part A and Part B by monitoring AEs, physical examination, vital signs, 12-lead ECGs, clinical laboratory values (hematology panel, multiphasic chemistry panel and urinalysis), and concomitant treatments. The C-SSRS assessment of suicidality risk is also be performed in Part B MAD.

In this study, AEs are reported for all subjects from the time of consent until the completion of the Follow-up visit. AEs reported prior to the first dose are denoted as pre-treatment. SAEs are reported for all subjects (enrolled and not enrolled) from the time of consent. AEs reported from the time of consent to confinement on Day −1 are recorded as pre-treatment AEs. Treatment-emergent AEs are evaluated from the first administration of study treatment until the Follow-up visit. AEs that are ongoing at the final onsite visit will be marked as Not Recovered/Not Resolved on the AE eCRF page.

All spontaneously volunteered and enquired for, as well as observed AEs, are recorded in the subject's medical records and the eCRF.

Given the nature of this compound, sedation is measured as a specific safety assessment using the Observer's Assessment of Alertness and Sedation (OAAS, Table 7; Höhener 2008) every two hours through 8 hours after administration of each dose, i.e., hours 2, 4, 6, and 8, in both Parts A and B of the study. If sedation is observed, it should also be reported as an adverse event.

TABLE 7
Observer's Assessment of Alertness/Sedation

| | Sedation | | | Facial | |
|---|---|---|---|---|---|
| Score | level | Responsiveness | Speech | expression | Eyes |
| 5 | Alert | Responds | Normal | Normal | Clear, |
| | | readily to name | | | no |
| | | | | | ptosis |
| 4 | Light | Lethargic | Mild slowing | Mild | Glazed |
| | | response to | | relaxation | or mild |
| | | name | | | ptosis |
| 3 | Moderate | Response only | Slurring or | Marked | Glazed |
| | | after name is | prominet | relaxation | and |
| | | called loudly | slowing | | marked |
| | | | | | ptosis |
| 2 | Deep | Responds only | Few | — | — |
| | | after mild | recognizable | | |
| | | prodding or | words | | |
| | | shaking | | | |
| 1 | Deep sleep, | Does not | — | — | — |
| | unconscious | respond to mild | | | |
| | | prodding or | | | |
| | | shaking | | | |

[from Höhener, D; Blumenthal, S; Borgeat, A (2008). Sedation and regional anaesthesia in the adult patient. British Journal of Anaesthesia, 100(1): 8-16.]

### Definition of Adverse Events

An AE is any event, side-effect, or other untoward medical occurrence that occurs in conjunction with the use of a medicinal product in humans, whether or not considered to have a causal relationship to this treatment. An AE can, therefore, be any unfavourable and unintended sign (that could include a clinically significant abnormal laboratory finding), symptom, or disease temporally associated with the use of a medicinal product, whether or not considered related to the medicinal product.

Events meeting the definition of an AE include:
Exacerbation of a chronic or intermittent pre-existing condition including either an increase in frequency and/or intensity of the condition.
New conditions detected or diagnosed after study treatment administration that occur during the reporting periods, even though it may have been present prior to the start of the study.
Signs, symptoms, or the clinical sequelae of a suspected interaction.
Signs, symptoms, or the clinical sequelae of a suspected overdose of either study treatment or concomitant medications (overdose per se will not be reported as an AE/SAE).

Events that do not meet the definition of an AE include:
Medical or surgical procedure (e.g, endoscopy, appendectomy); the condition that leads to the procedure should be reported as an AE if it meets the criteria of an AE.
Situations where an untoward medical occurrence did not occur (e.g., social and/or convenience admission to a hospital).
Anticipated day-to-day fluctuations of pre-existing disease(s) or condition(s) present or detected at the start of the study that do not worsen.

### Severity of an Adverse Event

Severity of AEs are graded by the Investigator as one of
Mild (Grade 1): A type of AE that is usually transient and may require only minimal treatment or therapeutic intervention. The event does not generally interfere with usual activities of daily living.
Moderate (Grade 2): A type of AE that is usually alleviated with additional specific therapeutic intervention. The event interferes with usual activities of daily living, causing discomfort but poses no significant or permanent risk of harm to the research subject.
Severe (Grade 3): A type of AE that interrupts usual activities of daily living, or significantly affects clinical status, or may require intensive therapeutic intervention.

Appendix 1 contains reference material from FDA “Guidance for Industry Toxicity Grading Scale for Healthy Adult and Adolescent Volunteers Enrolled in Preventive Vaccine Clinical Trials” from 2007 that provides AE grading guidelines.

### Causal Relationship of an Adverse Event

Relationship between study treatment and the occurrence of each AE is assessed. The following definitions are general guidelines to help assign grade of attribution:
NOT RELATED: An event that is clearly and incontrovertibly due to extraneous causes (disease, environment, etc).
UNLIKELY RELATED: An event where there is a small possibility that the event is likely to have been caused by the study treatment. The event may follow no known pattern of response and an alternative cause seems probable, but not definite. The AE has no clear temporal relationship to the study treatment and follows a known pattern of response, but a potential alternative cause is present.
POSSIBLY RELATED: An event where there is a reasonable possibility that the event might have been caused by study participation. A possibly related event may follow no known pattern of response and an alternative cause seems more likely. In other circumstances there may be significant uncertainty about the cause of the event, or a possible relationship to study participation cannot reasonably be ruled out.
PROBABLY RELATED: An event where there is a reasonable possibility that the event is likely to have been caused by the study treatment. The AE has a timely relationship to the study procedure(s) and follows a known pattern of response, but a potential alternative cause may be present.
DEFINITELY RELATED: An event that is clearly caused by the study treatment. A definitely related event has a strong temporal relationship, and an alternative cause is unlikely.

### Definition and Reporting of Serious Adverse Event

An SAE is an AE occurring during the study treatment that fulfils one or more of the following:
Results in death
It is immediately life-threatening
It requires in-patient hospitalisation or prolongation of existing hospitalisation
It results in persistent or significant disability or incapacity
Results in a congenital abnormality or birth defect
It is an important medical event that may jeopardise the subject or may require medical intervention to prevent one of the outcomes listed above

Important medical events that may not be one of the above may be considered an SAE by the Investigator when, based upon appropriate medical judgment, they are considered clinically significant and may jeopardise the subject, or may require medical or surgical intervention to prevent one of the outcomes listed above.

An AE is considered “life-threatening” if its occurrence places the subject at immediate risk of death. It does not include an AE that, had it occurred in a more severe form, might have caused death.

### Clinical Laboratory Abnormalities and Other Abnormal Assessments as Adverse Events and Serious Adverse Events

Abnormal laboratory findings (e.g., biochemistry, hematology, and urinalysis) or other abnormal assessments (e.g., ECG and vital signs) per se are not reported as AEs. However, those abnormal findings that are deemed clinically significant or are associated with signs and/or symptoms must be recorded as AEs if they meet the definition of an AE (and recorded as an SAE if they meet the criteria of being serious) as previously described. Clinically significant abnormal laboratory or other abnormal findings that are detected after consent or that are present at baseline and worsen after consent are included as AEs (and SAEs if serious).

### REFERENCES

Guidance for Industry: Toxicity Grading Scale for Healthy Adult and Adolescent Volunteers Enrolled in Preventive Vaccine Clinical Trials (September 2007) U.S. Department of Health and Human Services, Food and Drug Administration, Center for Biologics Evaluation and Research September 2007
Denes P, Larson J C, Lloyd-Jones D M. Prineas R J, Greenland P. Major and minor ECG abnormalities in asymptomatic women and risk of cardiovascular events and mortality. JAMA. 2007 Mar. 7; 297(9):978-85. doi: 10.1001/jama.297.9.978. PMID: 17341712.
Höhener, D; Blumenthal, S; Borgeat, A (2008). Sedation and regional anaesthesia in the adult patient. British Journal of Anaesthesia, 100(1):8-16.

### Appendix 1: Grade 3 Toxicity Reference Tool

The following tables should be utilized to assess adverse event severity and are excerpted from “Guidance for Industry: Toxicity Grading Scale for Healthy Adult and Adolescent Volunteers Enrolled in Preventive Vaccine Clinical Trials” issued September 2007) for use in grading toxicities.

A. Tables for Clinical Abnormalities

| | | | | Potentially Life |
|---|---|---|---|---|
| Local Reaction to | | | Severe | Threatening |
| Injectable Product | Mild (Grade 1) | Moderate(Grade 2) | (Grade 3) | (Grade 4) |
| Pain | Does not interfere | Repeated use of non- | Any use of | Emergency room |
| | with activity | narcotic pain | narcotic pain | (ER) visit or |
| | | reliever >24 hours or | reliever or | hospitalization |
| | | interferes with | prevents daily | |
| | | activity | activity | |
| Tenderness | Mild discomfort | Discomfort with | Significant | ER visit or |
| | to touch | movement | discomfort | hospitalization |
| | | | at rest | |
| Erythema/Redness * | 2.5-5 cm | 5.1-10 cm | >10 cm | Necrosis or |
| | | | | exfoliative |
| | | | | dermatitis |
| Induration/Swelling ** | 2.5-5 cm and | 5.1-10 cm or | >10 cm or | Necrosis |
| | does not interfere | interferes with | prevents daily | |
| | with activity | activity | activity | |

* In addition to grading the measured local reaction at the greatest single
diameter, the measurement should be recorded as a continuous variable.
** Induration/Swelling should be evaluated and graded using
the functional scale as well as the actual measurement.

| | | | | Potentially Life |
|---|---|---|---|---|
| Vital | Mild | | Severe | Threatening |
| Signs * | (Grade 1) | Moderate(Grade 2) | (Grade 3) | (Grade 4) |
| Fever (° C.) ** | 38.0-38.4 | 38.5-38.9 | 39.0-40 | >40 |
| (° F.) ** | 100.1-101.1 | 101.2-102.0 | 102.1-104 | >104 |
| Tachycardia - | 101-115 | 116-130 | >130 | ER visit or |
| beats | | | | hospitalization |
| per minute | | | | for arrhythmia |
| Bradycardia - | 50-54 | 45-49 | <45 | ER visit or |
| beats | | | | hospitalization |
| per minute*** | | | | for arrhythmia |
| Hypertension | 141-150 | 151-155 | >155 | ER visit or |
| (systolic) - | | | | hospitalization |
| mm Hg | | | | for malignant |
| | | | | hypertension |
| Hypertension | 91-95 | 96-100 | >100 | ER visit or |
| (diastolic) - | | | | hospitalization |
| mm Hg | | | | for malignant |
| | | | | hypertension |
| Hypotension | 85-89 | 80-84 | <80 | ER visit or |
| (systolic) - | | | | hospitalization |
| mm Hg | | | | for hypotensive |
| | | | | shock |
| Respiratory | 17-20 | 21-25 | >25 | Intubation\| |
| Rate - breaths | | | | |
| per minute | | | | |

* Subject should be at rest for all vital sign measurements.
** Oral temperature; no recent hot or cold beverages or smoking.
***When resting heart rate is between 60-100 beats per minute. Use clinical judgement when
characterizing bradycardia among some healthy subject populations, for example, conditioned athletes.

| | | | | Potentially Life |
|---|---|---|---|---|
| Systemic | Mild | | Severe | Threatening |
| (General) | (Grade 1) | Moderate(Grade 2) | (Grade 3) | (Grade 4) |
| Nausea/vomiting | No interference | Some interference | Prevents daily | ER visit or |
| | with activity or 1-2 | with activity or >2 | activity, requires | hospitalization |
| | episodes/24 hours | episodes/24 hours | outpatient IV | for hypotensive |
| | | | hydration | shock |
| Diarrhea | 2-3 loose stools | 4-5 stools or | 6 or more watery | ER visit or |
| | or <400 | 400-800 gms/24 | stools or >800 | hospitalization |
| | gms/24 hours | hours | gms/24 hours | |
| | | | or requires | |
| | | | outpatient IV | |
| | | | hydration | |
| Headache | No interference with | Repeated use of non- | Significant; any | ER visit or |
| | activity | narcotic pain | use of narcotic | hospitalization |
| | | reliever >24 hours or | pain reliever or | |
| | | some interference | prevents daily | |
| | | with activity | activity | |
| Fatigue | No interference with | Some interference with | Significant; | ER visit or |
| | activity | activity | prevents daily | hospitalization |
| | | | activity | |
| Myalgia | No interference with | Some interference with | Significant; | ER visit or |
| | activity | activity | prevents daily | hospitalization |
| | | | activity | |

| | | | | Potentially Life |
|---|---|---|---|---|
| | | | | Threatening |
| Systemic Illness | Mild (Grade 1) | (Moderate(Grade 2) | Severe (Grade 3) | (Grade 4) |
| Illness or clinical adverse | No interference | Some interference | Prevents daily | ER visit or |
| event (as defined | with activity | with activity not | activity and | hospitalization |
| according to applicable | | requiring medical | requires medical | |
| regulations) | | intervention | intervention | |

B. Tables for Laboratory Abnormalities
The laboratory values provided in the tables below serve as guidelines and are dependent upon institutional normal
parameters. Institutional normal reference ranges should be provided to demonstrate that they are appropriate.

| | | | | Potentially Life |
|---|---|---|---|---|
| | Mild | Moderate | Severe | Threatening |
| Serum * | (Grade 1) | (Grade 2) | (Grade 3) | (Grade 4)** |
| Sodium - | 132-134 | 130-131 | 125-129 | <125 |
| Hyponatremia mEq/L | | | | |
| Sodium - | 144-145 | 146-147 | 148-150 | >150 |
| Hypernatremia mEq/L | | | | |
| Potassium - | 5.1-5.2 | 5.3-5.4 | 5.5-5.6 | >5.6 |
| Hyperkalemia mEq/L | | | | |
| Potassium - | 3.5-3.6 | 3.3-3.4 | 3.1-3.2 | <3.1 |
| Hypokalemia mEq/L | | | | |
| Glucose - | 65-69 | 55-64 | 45-54 | <45 |
| Hypoglycemia mg/dL | | | | |
| Glucose - Hyperglycemia | | | | Insulin |
| Fasting - mg/dL | 100-110 | 111-125 | >125 | requirements or |
| Random - mg/dL | 110-125 | 126-200 | >200 | hyperosmolar |
| | | | | coma |
| Blood Urea Nitrogen | 23-26 | 27-31 | >31 | Requires |
| BUN mg/dL | | | | dialysis |
| Creatinine - mg/dL | 1.5-1.7 | 1.8-2.0 | 2.1-2.5 | >2.5 or requires |
| | | | | dialysis |
| Calcium - | 8.0-8.4 | 7.5-7.9 | 7.0-7.4 | <7.0 |
| hypocalcemia mg/dL | | | | |
| Calcium - | 10.5-11.0 | 11.1-11.5 | 11.6-12.0 | >12.0 |
| hypercalcemia mg/dL | | | | |
| Magnesium - | 1.3-1.5 | 1.1-1.2 | 0.9-1.0 | <0.9 |
| hypomagnesemia mg/dL | | | | |
| Phosphorous - | 2.3-2.5 | 2.0-2.2 | 1.6-1.9 | <1.6 |
| hypophosphatemia mg/dL | | | | |
| CPK - mg/dL | 1.25-1.5 × | 1.6-3.0 × ULN | 3.1-10 × ULN | >10 × ULN |
| | ULN*** | | | |
| Albumin - | 2.8-3.1 | 2.5-2.7 | <2.5 | — |
| Hypoalbuminemia g/dL | | | | |
| Total Protein - | 5.5-6.0 | 5.0-5.4 | <5.0 | — |
| Hypoproteinemia g/dL | | | | |
| Alkaline phosphate - | 1.1-2.0 × ULN | 2.1-3.0 × ULN | 3.1-10 × ULN | >10 × ULN |
| increase by factor | | | | |
| Liver Function Tests-ALT, | 1.1-2.5 × ULN | 2.6-5.0 × ULN | 5.1-10 × ULN | >10 × ULN |
| AST increase by factor | | | | |
| Bilirubin - when | 1.1-1.25 × ULN | 1.26-1.5 × ULN | 1.51-1.75 × ULN | >1.75 × ULN |
| accompanied by | | | | |
| any increase in | | | | |
| Liver Function Test | | | | |
| increase by factor | | | | |
| Bilirubin - when Liver | 1.1-1.5 × ULN | 1.6-2.0 × ULN | 2.0-3.0 × ULN | >3.0 × ULN |
| Function Test is normal; | | | | |
| increase by factor | | | | |
| Cholesterol | 201-210 | 211-225 | >226 | — |
| Pancreatic enzymes - | 1.1-1.5 × ULN | 1.6-2.0 × ULN | 2.1-5.0 × ULN | >5.0 × ULN |
| amylase, lipase | | | | |

* The laboratory values provided in the tables serve as guidelines and are dependent upon institutional normal
parameters. Institutional normal reference ranges should be provided to demonstrate that they are appropriate.
**The clinical signs or symptoms associated with laboratory abnormalities might result in characterization of the laboratory
abnormalities as Potentially Life Threatening (Grade 4). For example, a low sodium value that falls within a grade 3 parameter (125-129 mE/L)
should be recorded as a grade 4 hyponatremia event if the subject had a new seizure associated with the low sodium value.
***ULN [symbol] is the upper limit of the normal range.

| | | | | Potentially Life |
|---|---|---|---|---|
| | Mild | Moderate | Severe | Threatening |
| Hematology * | (Grade 1) | (Grade 2) | (Grade 3) | (Grade 4) |
| Hemoglobin | 11.0-12.0 | 9.5-10.9 | 8.0-9.4 | <8.0 |
| (Female) - gm/dL | | | | |
| Hemoglobin (Female) | Any | 1.6-2.0 | 2.1-5.0 | >5.0 |
| change from baseline | decrease-1.5 | | | |
| value - gm/dL | | | | |
| Hemoglobin | 12.5-13.5 | 10.5-12.4 | 8.5-10.4 | <8.5 |
| (Male) - gm/dL | | | | |
| Hemoglobin (Male) | Any | 1.6-2.0 | 2.1-5.0 | >5.0 |
| change from baseline | decrease-1.5 | | | |
| value - gm/dL | | | | |
| WBC Increase - cell/mm³ | 10,800-15,000 | 15,001-20,000 | 20,001-25,000 | >25,000 |
| WBC Decrease - cell/mm³ | 2,500-3,500 | 1,500-2,499 | 1,000-1,499 | <1,000 |
| Lymphocytes | 750-1,000 | 500-749 | 250-499 | <250 |
| Decrease - cell/mm³ | | | | |
| Neutrophils | 1,500-2,000 | 1,000-1,499 | 500-999 | <500 |
| Decrease - cell/mm³ | | | | |
| Eosinophils - cell/mm³ | 650-1500 | 1501-5000 | >5000 | Hypereosinophilic |
| Platelets Decreased - cell/mm³ | 125,000-140,000 | 100,000-124,000 | 25,000-99,000 | <25,000 |
| PT - increase by factor | 1.0-1.10 × | 1.11-1.20 × ULN | 1.21-1.25 × ULN | >1.25 × ULN |
| (prothrombin time) | ULN*** | | | |
| PTT - increase by factor | 1.0-1.2 × ULN | 1.21-1.4 × ULN | 1.41-1.5 × ULN | >1.5 × ULN |
| (partial thromboplastin time) | | | | |
| Fibrinogen increase - mg/dL | 400-500 | 501-600 | >600 | — |
| Fibrinogen decrease - mg/dL | 150-200 | 125-149 | 100-124 | <100 or associated |
| | | | | with gross bleeding |
| | | | | or disseminated |
| | | | | intravascular |
| | | | | coagulation (DIC) |

* The laboratory values provided in the tables serve as guidelines and are dependent upon institutional normal
parameters. Institutional normal reference ranges should be provided to demonstrate that they are appropriate.
** [symbol] ULN [symbol] is the upper limit of the normal range.

| | | | | Potentially Life |
|---|---|---|---|---|
| | | Moderate | | Threatening |
| Urine * | Mild (Grade 1) | (Grade 2) | Severe (Grade 3) | (Grade 4) |
| Protein | Trace | 1+ | 2+ | Hospitalaztion or |
| | | | | dialysis |
| Glucose | Trace | 1+ | 2+ | Hospitalization for |
| | | | | hyperglycemia |
| Blood (microscopic) - | 1-10 | 11-50 | >50 and/or gross | Hospitalization or |
| red blood cells per | | | blood | packed red blood |
| high power field (rbc/hpf) | | | | cells (PRBC) |
| | | | | transfusion |

* The laboratory values provided in the tables serve as guidelines and are dependent upon institutional normal parameters. Institutional normal reference ranges should be provided to demonstrate that they are appropriate. [symbol]
indicates data missing or illegible when filed

### Appendix 2: Blood Volumes

Estimates for the total volumes of blood to be collected per subject in Part A and Part B are presented in Appendix Table 1 and Appendix Table 2 respectively.

APPENDIX TABLE 1
Estimated Blood Volumes in Part A

| | | Total Number | | |
|---|---|---|---|---|
| Test | Per Test Volume | of Samples | Total Volume | |
| HIV, hepatitis B | Two tubes, 3.5 mL | 1 | 8.5 | mL |
| & C blood screen | | | | |
| Pregnancy test | From chemistry tube | | 0 | |
| and/or FSH (as | | | | |
| applicable) | | | | |
| Blood Chemistry | 2.7 mL | 6 | 16.2 | mL |
| Hematology | 4 mL | 6 | 24 | mL |
| PK | 4 mL | 18 | 72 | mL |

APPENDIX TABLE 2
Estimated Blood Volumes in Part B

| | | Total Number | | |
|---|---|---|---|---|
| Test | Per Test Volume | of Samples | Total Volume | |
| HIV, hepatitis B | Two tubes, 3.5 mL | 1 | 8.5 | mL |
| & C blood screen | | | | |
| Pregnancy test | From chemistry tube | | 0 | |
| and/or FSH (as | | | | |
| applicable) | | | | |
| Blood Chemistry | 2.7 mL | 3 | 8.1 | mL |
| Hematology | 4 mL | 3 | 12 | mL |
| PK | 4 mL | 40 | 160 | mL |

### Example 2: Salt Screen of Compound I Free-Base

The present disclosure describes a salt screen performed using Compound I:

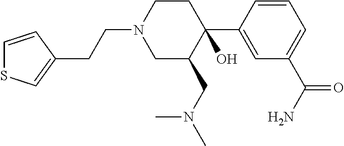 [SMILES: CN(C)C[C@H]1CN(CCc2ccsc2)CC[C@]1(O)c1cccc(C(N)=O)c1]

Thirty different salt forming acids and several potential co-formers were investigated. Characterization of salts produced at a small scale allowed the selection of one salt, which was scaled up and subjected to a polymorph screen.

All crystalline salts isolated during the experiments described herein were hydrates and/or solvates. Whereas the sulfate salt was crystalline, had high solubility in unbuffered water, and was extremely hygroscopic, the diphosphate salt was crystalline, had high solubility in unbuffered water, and was moderately hygroscopic. Other salts, namely the glycolate, acetate and dibenzoate salts, possessed either low thermal stability or were difficult to isolate as a crystalline solid. During process development, it was found that the sulfate salt was not easily isolable as a workable solid due to its hygroscopicity. The diphosphate salt, despite being a monohydrate, proved to be more stable and therefore more processable. Thus, in certain preferred embodiments, the Compound I salt is the diphosphate salt of Compound I.

The salt and/or polymorph solid forms described herein were characterized using methods common to those in the art, including powder X-ray diffraction (XRD), Nuclear Magnetic Resonance Spectroscopy (NMR), Dynamic Vapor Sorption (DVS), Differential Scanning Calorimetry (DSC), and Thermogravimetric Analysis (TGA).

### Materials and Methods

### Differential Scanning Calorimetry (DSC)

DSC data were collected on a TA Instruments Q2000 DSC. In general, samples in the mass range of 1 to 10 mg were crimped in aluminum sample pans and scanned from 25 to 300° C. or 300° C. at 10° C./minute using a nitrogen purge of 50 mL/min.

### Thermogravimetric Analysis (TGA)

TGA data were collected on a TA Instruments Q500 TGA. In general, samples in the mass range of 2 to 15 mg were placed in an open, pre-tared platinum sample pan and attached by fine wire to a microbalance. The sample was suspended in a furnace, which was heated from 25 to about 300° C. at 10° C./min using a nitrogen purge at 100 mL/min. The sample weight change as a function of temperature was observed.

### Proton Nuclear Magnetic Resonance Spectroscopy (¹H-NMR)

The samples were prepared by dissolving 1 to 10 mg of the API in deuterated DMSO with 0.05% (v/v) tetramethylsilane (TMS). The spectra were collected at ambient temperature on a Bruker 400 MHz NMR spectrometer.

### X-Ray Powder Diffraction (XRD)

X-ray powder diffraction patterns were obtained using a Bruker D8 Discovery diffractometer equipped with an XYZ stage, laser video microscope for positioning and a Vantec 500 (Photon) detector. Collection times were nominally 120 seconds. A Cu Kα radiation 1.5406 angstrom source operating at 40 kV and 40 mA was used to irradiate the samples. The X-ray optics consists of a Gobel mirror coupled with a pinhole collimator of 0.5 mm. Theta—theta continuous scans were employed with a sample-detector distance of 30 cm, which gives an effective 20 range of 4 to 40°2θ. Samples were mounted on a low background quartz plates.

### Hygroscopicity—Dynamic Vapor Sorption (DVS)

DVS is a gravimetric technique that measures how quickly and how much of a solvent (water) is sorbed by a sample. The relative humidity or vapor concentration surrounding the sample is varied while the change in mass of the sample is measured. A vapor sorption isotherm shows the equilibrium amount of vapor sorbed as a function of relativity humidity. The mass values at each relative humidity step are used to generate an isotherm. Isotherms are divided in two components: sorption for increasing humidity steps and desorption for decreasing humidity steps. A plot of kinetic data is also used to show the change in mass and humidity as a function of time.

The samples were analyzed using a TA Q5000 automated dynamic vapor sorption analyzer. The samples were dried at 60° C. over 180 minutes and then cooled to 25° C. with a dry nitrogen purge. The samples were then subjected to 0 to 90% RH, back to 10% RH at 25° C. in 10% RH steps.

### Characterization of Supplied Material (i.e., Compound I Free-Base)

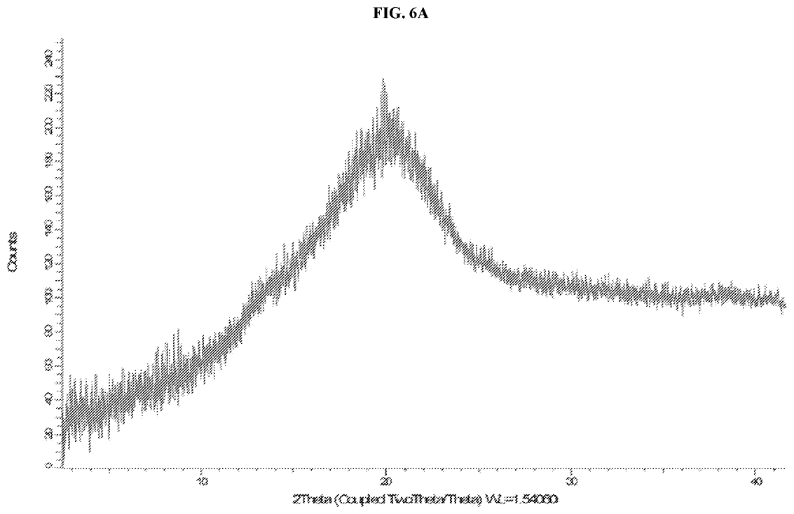

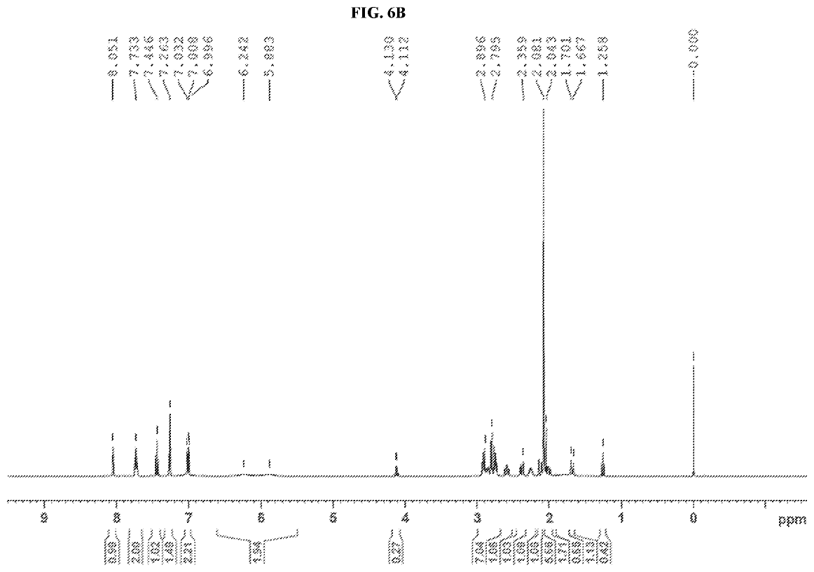

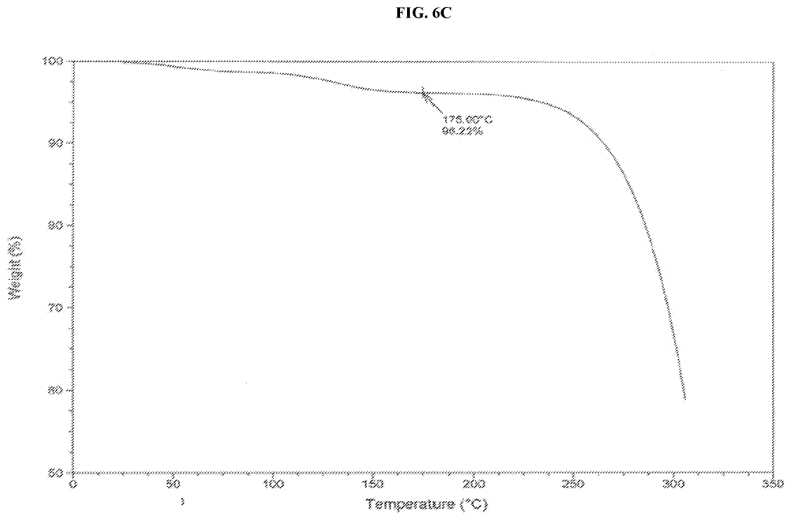

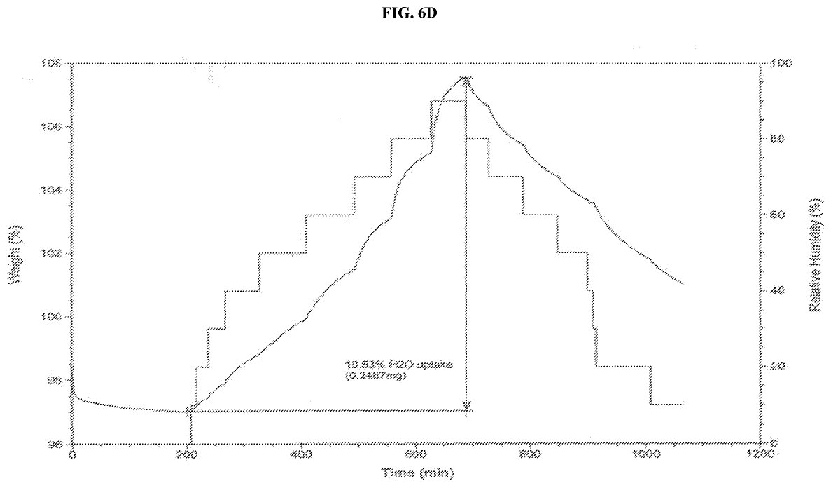

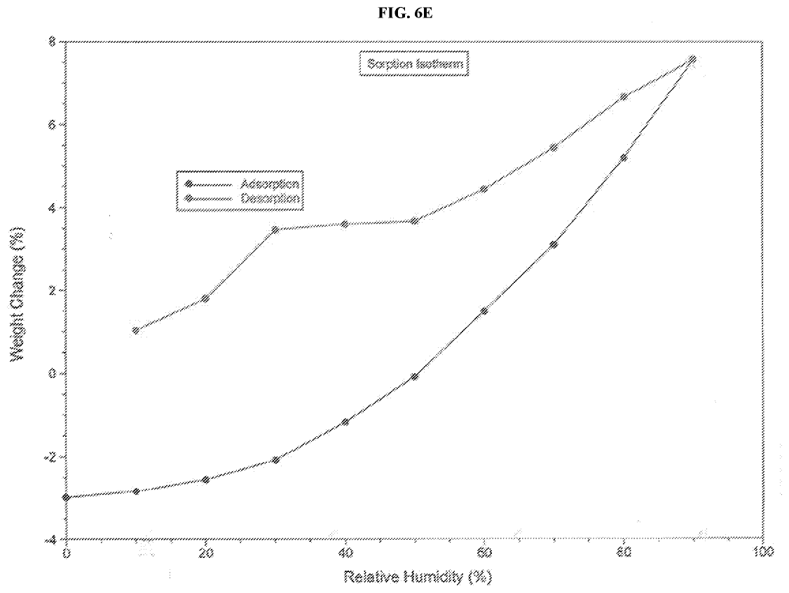

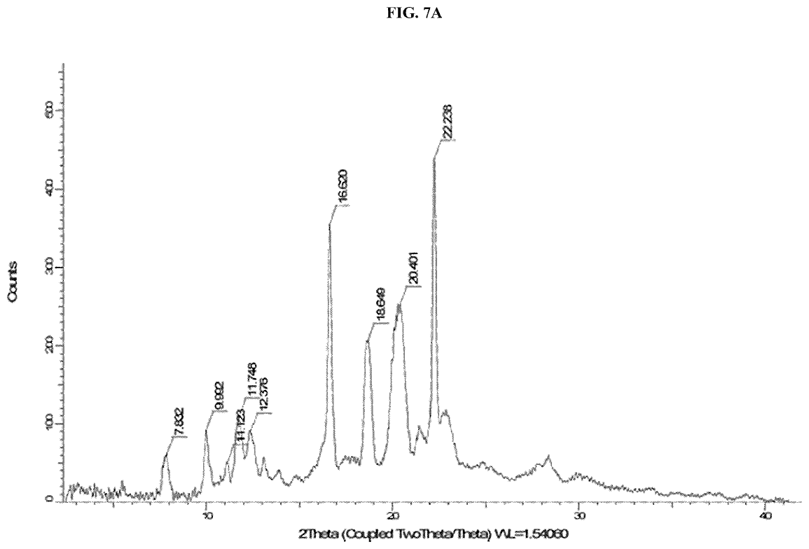

The starting material used in the experiments described herein is the Compound I free base (i.e., 3-((3S,4R)-3-((dimethylamino)methyl)-4-hydroxy-1-(2-(thiophen-3-yl)ethyl)piperidin-4-vl)benzamide). The starting material was characterized by XRD, NMR, TGA, and DVS. X-ray powder diffraction was used to examine the supplied lot to determine if it was crystalline. XRD analysis of Compound I free base indicated it was amorphous, as shown in FIG. 6A. The NMR spectrum of Compound I free base is shown in FIG. 6B and is consistent with the expected structure of Compound I. The TGA profile shows a 3.78 wt % mass loss from 25 to 175° C. and is shown in FIG. 6C. Further, A DVS scan of Compound I free base showed it was hygroscopic with a maximum moisture uptake of approximately 10.5 wt % (FIG. 6D). The sample exhibited some hysteresis, as shown in the isotherm plot in FIG. 6E.

### Primary Salt Screen

Thirty acids were used during in the salt screen described herein (Table 8). The acids can be divided into three types of classes for use in drug substances (i.e., Classes I-III). In certain embodiments, Class I salt formers are those that have unrestricted use because they form physiologically ubiquitous ions or they occur as intermediate metabolites in biochemical pathways. In certain embodiments, Class II salt formers are not naturally occurring, but have shown to exhibit low toxicity and good tolerability. In certain embodiments, Class III salt formers show safety status that is somewhat less desirable and is less advisable for general use.

TABLE 8
Salt forming acids

| Acid | Salt | pKa | Class |
|---|---|---|---|
| Adipic acid | Adipate | 4.4, 5.4 | I |
| Aspartic acid | Aspartate | 1.9, 3.7, 9.6 | I |
| Benzoic acid | Benzoate | 4.2 | II |
| Benzenesulfonic acid | Besylate | 0.7 to 1.6 | II |
| Camphorsulfonic acid | Camsylate | 2.2 | III |
| Citric acid | Citrate | 3.1, 4.8, 6.4 | I |
| 1,2-Ethanedisulfonic acid | Edisylate | −2.1, −1.5 | II |
| Ethanesulfonic acid | Esylate | −2.1 | II |
| Fumaric acid | Fumarate | 3.0, 4.4 | I |
| Gluconic acid | Gluconate | 3.8 | I |
| Glutamic acid | Glutamate | 2.2, 4.3, 9.7 | I |
| Glycolic acid | Glycolate | 3.3 | I |
| Hippuric acid | Hippurate | 3.6 | I |
| Hydrochloric acid | Hydrochloride | −6 to −3 | I |
| Malic acid | Malate | 3.5, 5.1 | I |
| Maleic acid | Maleate | 1.9, 6.2 | I |
| Methanesulfonic acid | Mesylate | −1.2 | II |
| Mucic acid | Mucate | 3.1, 3.6 | I |
| 1,5-Naphthalenedisulfonic acid | Napadisylate | −3.4, −2.6 | II |
| Naphthalene-2-sulfonic acid | Napsylate | 0.2 | II |
| Nicotinic acid | Nicotinate | 4.9 | II |
| Oxalic acid | Oxalate | 1.3, 4.3 | II |
| Pamoic acid | Pamoate | 2.5, 3.1 | II |
| Phosphoric acid | Phosphate | 2.0, 7.2, 12.4 | I |
| Sebacic acid | Sebacate | 4.6, 5.6 | I |
| Stearic acid | Stearate | 4.9 | I |
| Sulfuric acid | Sulfate | −3.0, 1.9 | I |
| Tartaric acid | Tartrate | 3.0, 4.3 | I |
| p-Toluenesulfonic acid | Tosylate | −0.5 | II |

The salt formation experiments were prepared at a 40-80 mg scale. In the first panel, the free base was placed in a test tube and salt forming acid solutions (0.1 M, 2 mL in methanol) were added. All experiments contained 1:1 molar ratios of the acid to free base. Extra solvent (~7 mL methanol) was added to increase the volume each solution. The samples were crystallized by evaporation under nitrogen purge (~2 psi) at ambient temperature. Almost every sample yielded amorphous solids, which were later reconstituted in water and evaporated under a nitrogen purge. Two samples had unique diffraction patterns (i.e., sulfate from methanol and acetate from water) while the rest were either weakly crystalline, amorphous solids, or glasses. The results of Panel 1 are summarized in Table 9.

TABLE 9
Summary of results for crystallization panel 1

| | Recryst. from | Recryst. from |
|---|---|---|
| Acid | MeOH | water |
| Citric | Amorphous solid | Glass |
| L-Tartaric | Amorphous solid | Glass |
| Oxalic | Amorphous solid | Glass |
| Benzoic | Amorphous solid | Glass |
| Succinic | Amorphous solid | Glass |
| Maleic | Amorphous solid | Glass |
| Fumaric | Amorphous solid | Glass |
| Phosphoric | Amorphous solid | Glass |
| Benzenesulfonic | Amorphous solid | Glass |
| L-Malic | Amorphous solid | Glass |
| Acetic | Amorphous solid | Crystalline solid |
| p-Toluenesulfonic | Amorphous solid | Glass |
| HCl | Amorphous solid | Amorphous solid |
| Naphthalene-2-sulfonic | Amorphous solid | Glass |
| Methanesulfonic | Amorphous solid | Glass |
| Ethanesulfonic | Amorphous solid | Glass |
| Sulfuric | Crystalline solid | Amorphous solid |
| Sulfuric (0.5 eq) | Amorphous solid | Amorphous solid |
| Ethanedisulfonic | Amorphous solid | Amorphous solid |
| Ethanedisulfonic (0.5 eq) | Amorphous solid | Weakly |
| | | crystalline solid |
| Naphthalenedisulfonic | Amorphous solid | Glass |
| Naphthalenedisulfonic (0.5 eq) | Amorphous solid | Glass |

In Panel 2, each sample was crystallized from 80/20 mixture of acetonitrile/methanol (Table 10). Crystalline solids were isolated from experiments with sulfuric and succinic acid, while every other sample yielded glasses or amorphous solids.

TABLE 10
Summary of results for crystallization panel 2

| Acid | Result |
|---|---|
| Methanesulfonic | Glass |
| Ethanesulfonic | Glass |
| Naphthalene-2-sulfonic | Glass |
| Acetic | Glass |
| Phosphoric | Amorphous solid |
| p-Toluenesulfonic | Glass |
| HCl | Glass |
| Benzenesulfonic | Glass |
| L-Malic | Amorphous solid |
| Citric | Amorphous solid |
| L-Tartaric | Amorphous solid |
| Fumaric | Glass |
| Oxalic | Glass |
| Benzoic | Glass |
| Succinic | Weakly crystalline solid |
| Maleic | Glass |
| Sulfuric | Crystalline solid |
| Sulfuric (0.5 eq) | Crystalline solid |
| Ethanedisulfonic | Amorphous solid |
| Ethanedisulfonic (0.5 eq.) | Amorphous solid |
| Naphthalenedisulfonic | Amorphous solid |
| Naphthalenedisulfonic (0.5 eq.) | Amorphous solid |

Panel 3 was carried out using two equivalents of acid in order to explore the possible formation of di-salts (Table 11). Evaporation from methanol yielded one crystalline solid, the diphosphate monohydrate salt, while the rest resulted in glasses. However, the dibenzoate salt eventually crystallized as a monohydrate after sitting in the fume hood for an extended period of time (i. e., a period of several weeks).

TABLE 11
Summary of results for crystallization panel 3

| Acid | Result |
|---|---|
| Sulfuric | Glass |
| Succinic | Glass |
| Benzoic | Crystalline solid* |
| L-Tartaric | Glass |
| Benezenesulfonic | Glass |
| p-Toluenesulfonic | Glass |
| Oxalic | Glass |
| L-Malic | Glass |
| HCl | Glass |
| Maleic | Glass |
| Fumaric | Glass |
| Citric | Glass |
| Phosphoric | Crystalline solid |
| Methanesulfonic | Glass |
| Naphthalene-2-sulfonic | Glass |
| Ethanesulfonic | Glass |
| Acetic | Glass |
| Ethanedisulfonic | Glass |
| Naphthalenedisulfonic | Glass |

*initially a glass, with crystallization occurring after several weeks.

In Panel 4, the salt library was expanded to include bases, potential co-crystal formers, and acids which are infrequently used in salt formation experiments. All experiments in Panel 4 were performed at 1:1 stoichiometry. The only notable discovery in Panel 4 was the glycolate salt, which was isolated as a hydrate. The results of Panel 4 are summarized in Table 12.

TABLE 12
Summary of results for crystallization panel 4

| Acid/Base/Co-Former | Solvent | Result |
|---|---|---|
| L-Glutamic acid | Water | Amorphous solid |
| D-Glutamic acid | Water | Weakly crystalline |
| | | solid |
| Mucic acid | Water | Amorphous solid |
| L-Aspartic acid | Water | Weakly crystalline |
| | | solid |
| Hippuric acid | Water | Glass |
| Glycolic acid | Water | Crystalline solid |
| (S)-Camphor-10- | Water | Glass |
| sulfonic acid | | |
| (R)-Camphor-10- | Water | Glass |
| sulfonic acid | | |
| D-Gluconic acid | Water | Glass |
| Sodium hydroxide | Water | Glass |
| Potassium hydroxide | Water | Amorphous solid |
| Adipic acid | Ethanol | Glass |
| Stearic acid | Ethanol | Glass |
| Aspartame | Ethanol/water | Glass |
| | (1:1) | |
| Ferulic acid | Ethanol | Glass |
| Saccharin | Ethanol | Glass |
| Sebacic acid | Ethanol | Glass |
| Methyl | Ethanol | Glass |
| 4-hydroxybenzoate | | |
| D-Mannitol | Ethanol/water | Crystalline solid |
| | (9:1) | (D-Mannitol) |
| Nicotinic acid | Ethanol | Glass |
| Pamoic acid | DMF | Amorphous solid |

In an attempt to discover an anhydrous salt form, the four crystalline salts which were isolated as hydrates (i.e., acetate, phosphate, sulfate, and glycolate crystalline salts) were recrystallized from a variety of organic solvents, along with the amorphous HCl salt. The results are presented in Table 13. No new forms were discovered in the recrystallization panel, as only the starting material or amorphous solids were recovered.

TABLE 13
Summary of results for miscellaneous recrystallization experiments

| Salt | Solvent | Result |
|---|---|---|
| Diphosphate | DMF | Crystalline solid* |
| Diphosphate | THF | Crystalline solid* |
| Diphosphate | Acetone | Crystalline solid* |
| Diphosphate | Trifluoroethanol | Amorphous solid |
| Diphosphate | Toluene | Crystalline solid* |
| Diphosphate | Ethyl acetate | Crystalline solid* |
| Diphosphate | Methylene chloride | Crystalline solid* |
| Diphosphate | Acetonitrile | Crystalline solid* |
| HCl | DMF | Glass |
| HCl | THF | Not enough solid |
| | | recovered |
| HCl | Acetone | Not enough solid |
| | | recovered |
| HCl | Trifluoroethanol | Glass |
| HCl | Toluene | Amorphous solid |
| HCl | Ethyl acetate | Glass |
| HCl | Methylene chloride | Amorphous solid |
| HCl | Acetonitrile | Amorphous solid |
| Glycolate | DMF | Glass |
| Glycolate | THF | Glass |
| Glycolate | Acetone | Glass |
| Acetate | DMF | Glass |
| Acetate | THF | Glass |
| Acetate | Acetone | Glass |
| Acetate | DMA | Glass |
| Acetate | Toluene | Crystalline solid* |
| Acetate | Acetonitrile | Glass |
| Sulfate | DMF | Crystalline solid* |
| Sulfate | THF | Crystalline solid* |
| Sulfate | Acetone | Crystalline solid* |
| Sulfate | DMA | Amorphous solid |

*Same diffraction pattern as starting material.

### Characterization of Salts

### Compound I Acetate

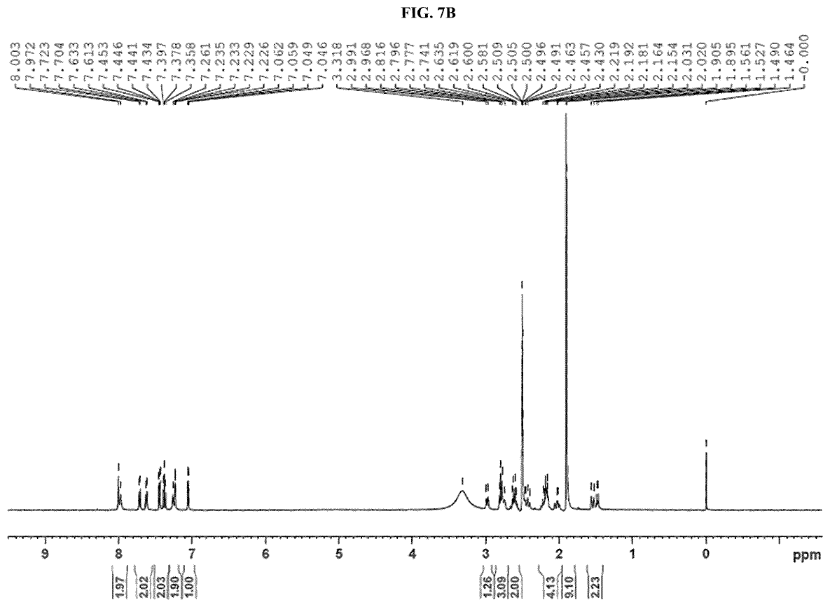

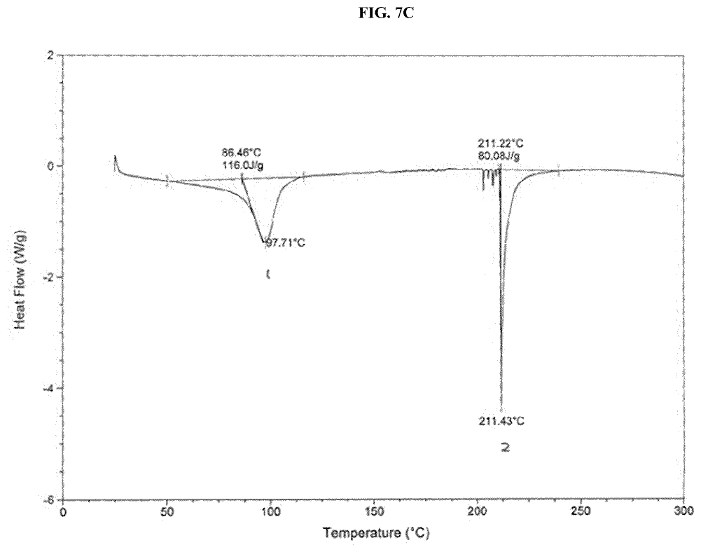

The XRD pattern of the acetate salt indicated the batch was partially crystalline as shown in FIG. 7A. The NMR spectrum of the acetate salt was consistent with the protonated API and showed 3 molar equivalents of acetic acid, despite the fact that only 1 equivalent of acetic acid was used to make the salt (FIG. 7B). The DSC profile exhibited a sharp melting endotherm at an onset of 211.2° C., peak temperature of 211.4° C. and estimated enthalpy of fusion of 80.1 J/g. The melting endotherm was preceded by a broad desolvation endotherm between 75° C. and 110° C. (FIG. 7C).

On the basis of the DSC data, the acetate salt may be a hydrate. Additionally, the acetate salt had low thermal stability, as it decomposed after heating overnight at 105° C. in an oven.

### Compound I Sulfate

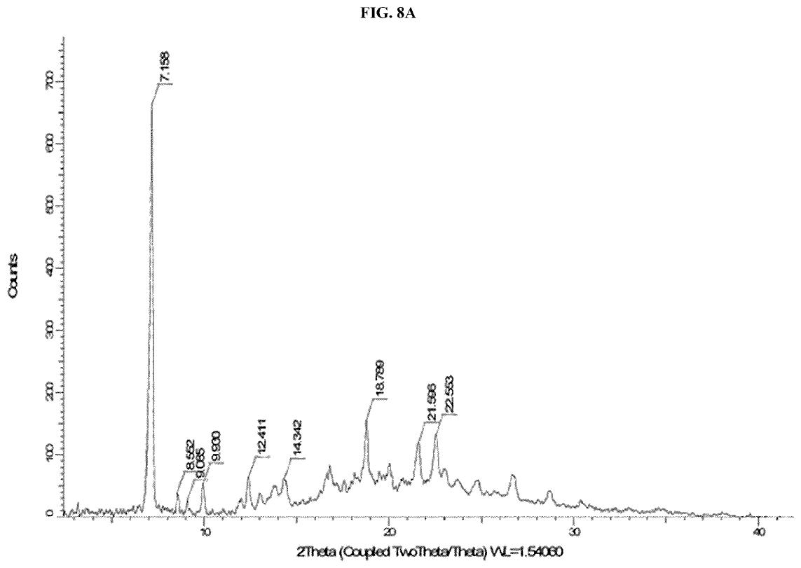

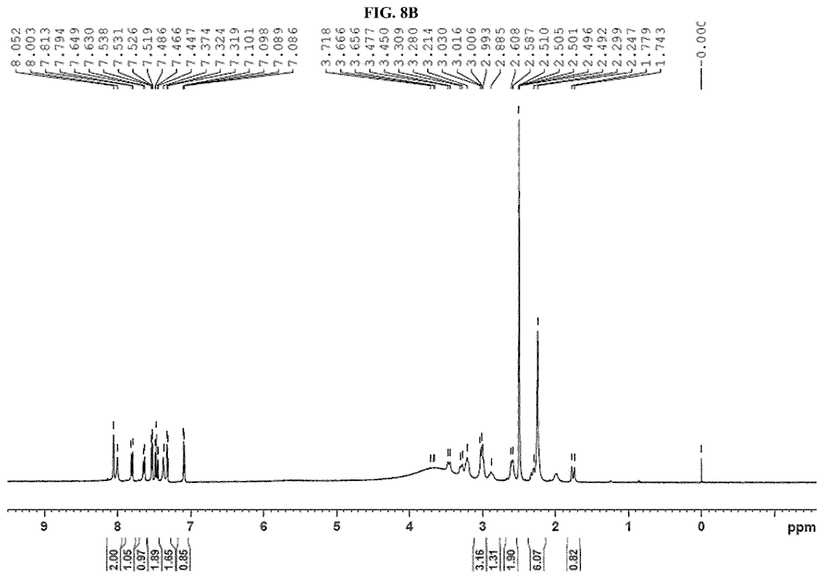

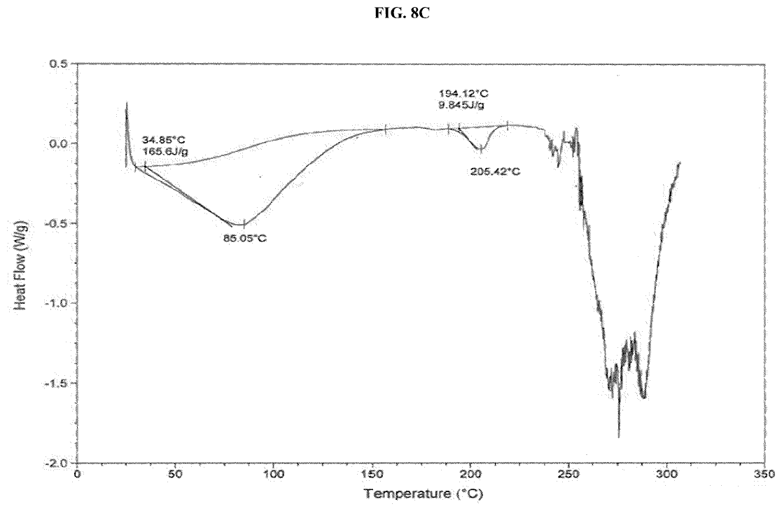

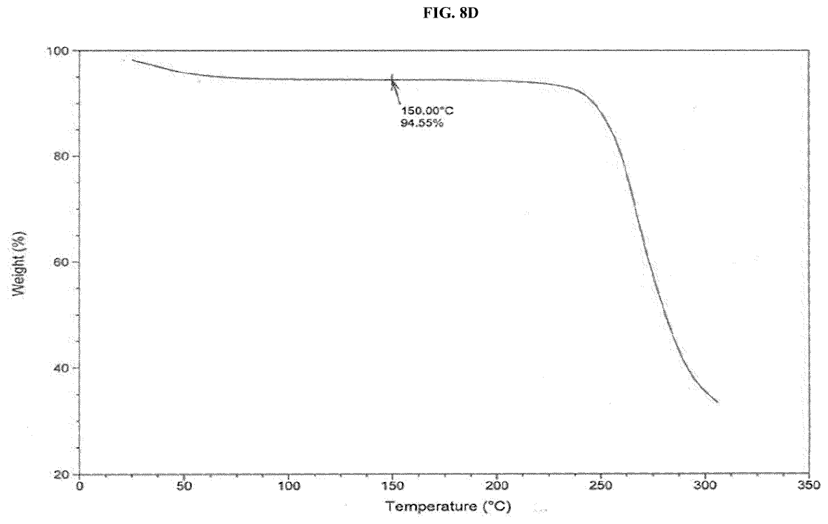

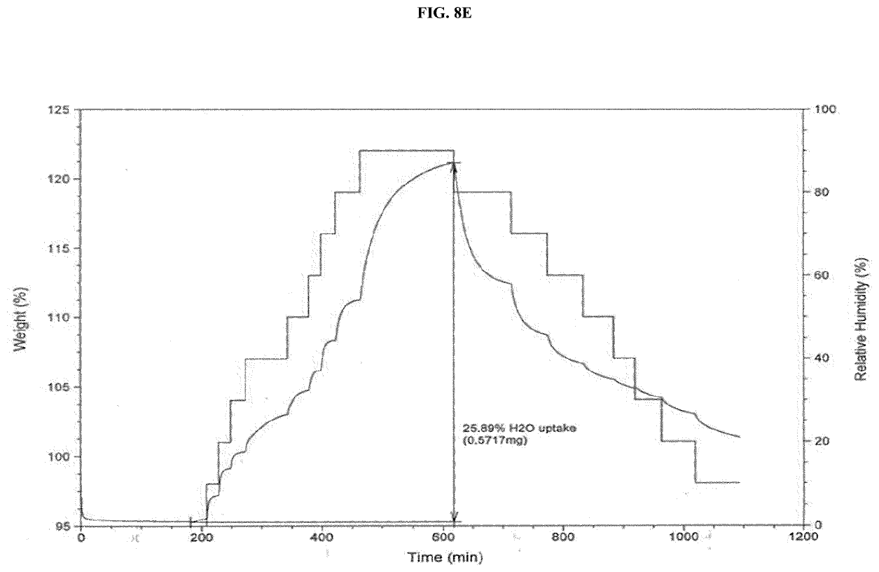

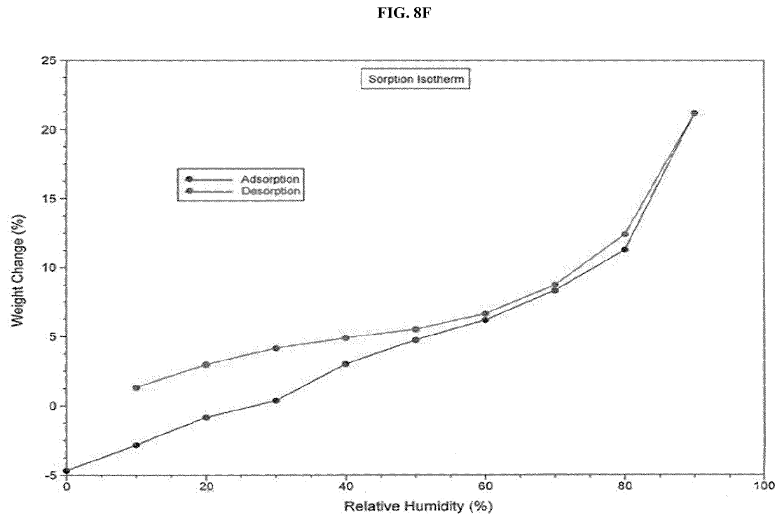

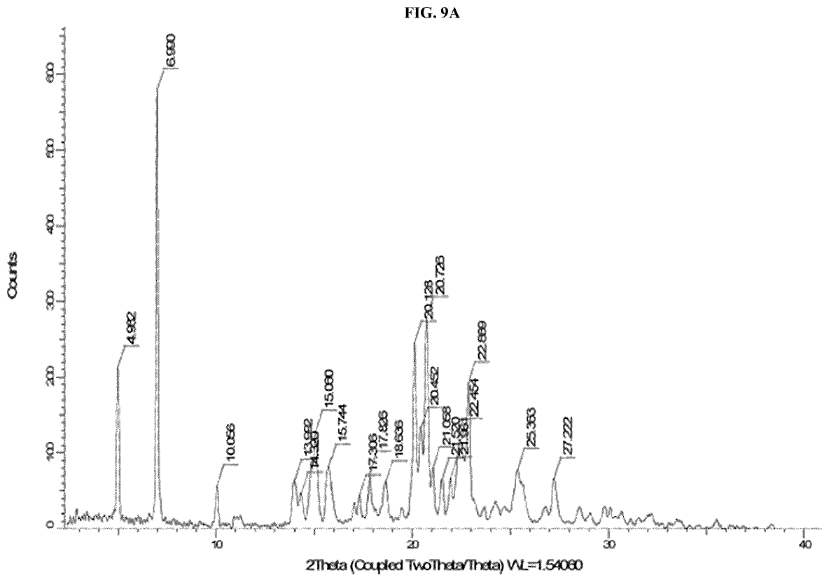

The XRD pattern of the sulfate salt indicated the batch was a partially crystalline solid as shown in FIG. 8A.

The NMR spectrum of the sulfate salt is consistent with the protonated API with no residual solvents except water (FIG. 8B). The DSC profile exhibited two broad endotherms with peak maxima at 85° C. and 205° C. (FIG. 8C), followed by decomposition. The TGA profile shows 5.45 wt % loss from 25° C. to 150° C. (FIG. 8D). DVS data showed the salt was extremely hygroscopic, with a maximum water uptake of 25.9 wt % at 90% RH (FIG. 8E). No hysteresis was observed in the isotherm plot, as shown in FIG. 8F.

The sulfate salt was isolated as a hydrate as well as ethanol and methanol solvates. A crystalline hydrate of the sulfate salt could not be reliably reproduced under process conditions due to its tendency to deliquesce when filtered.

### Compound I Diphosphate

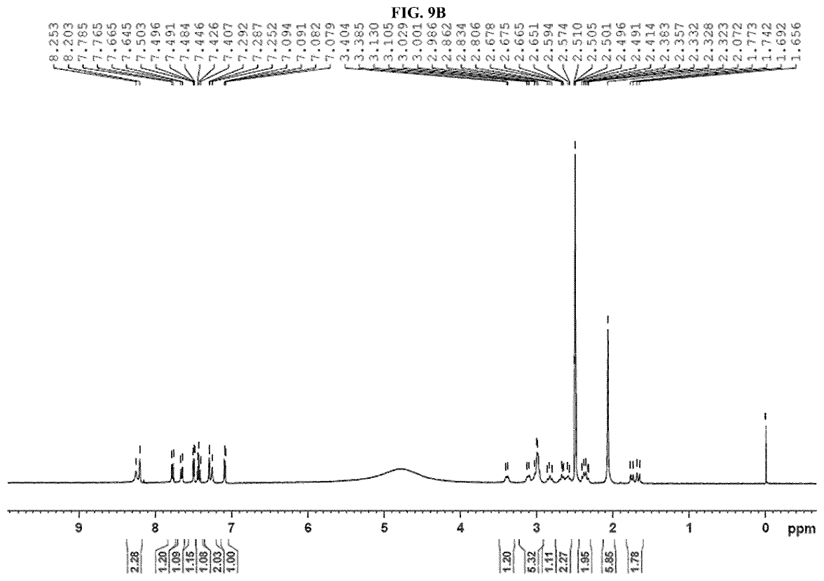

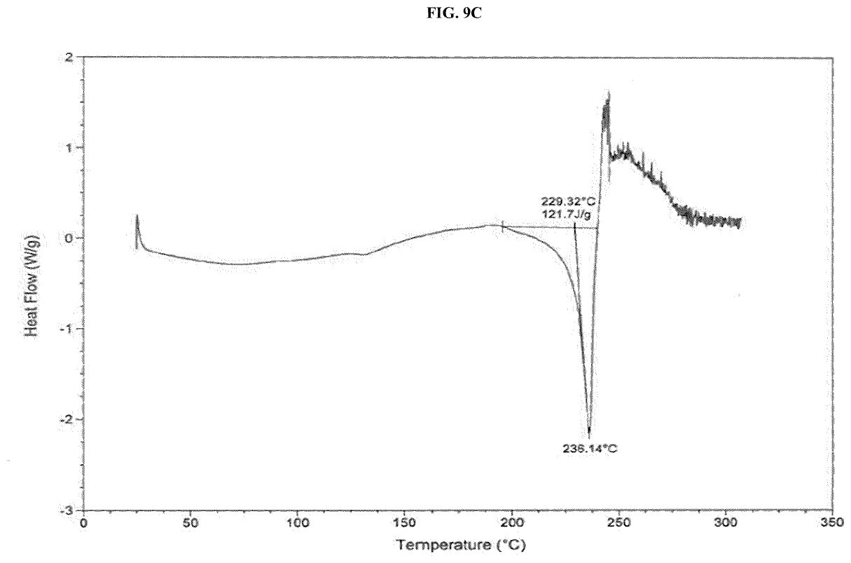

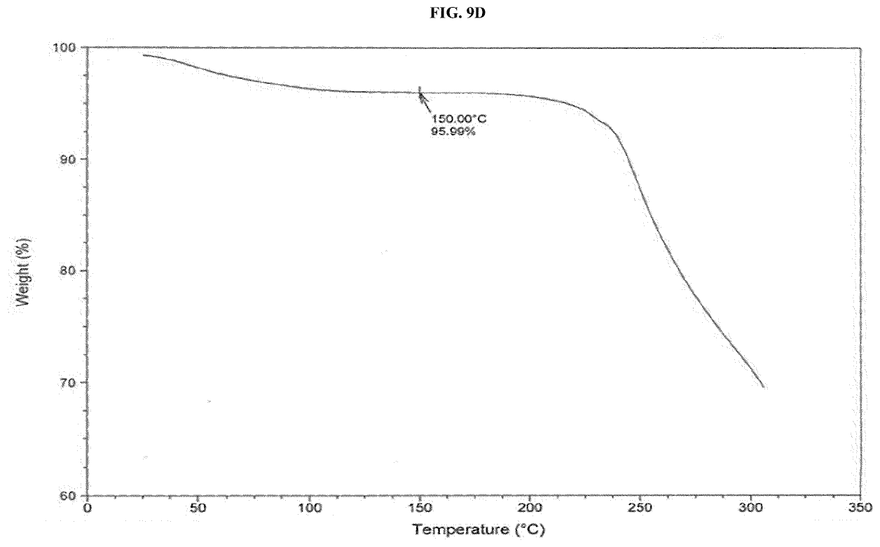

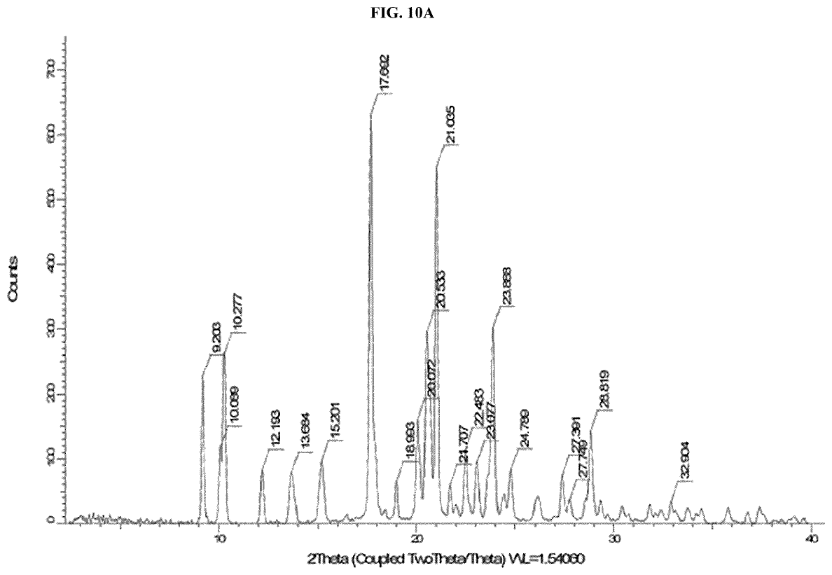

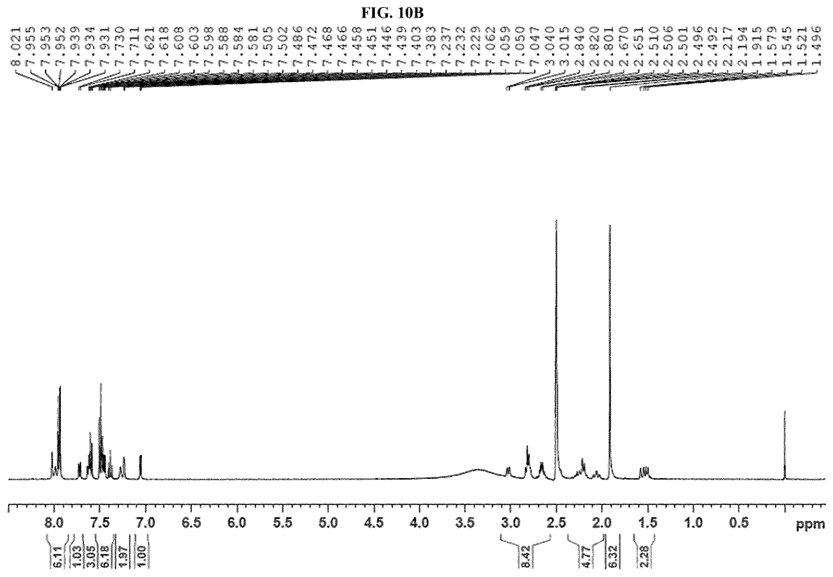

The XRD pattern of the diphosphate salt indicated the batch was a crystalline solid as shown in FIG. 9A. The ¹H NMR spectrum of the phosphate salt is consistent with the expected structure and is shown in FIG. 9B. ³¹P NMR was used to confirm the stoichiometry of the salt (found 31.1% H₃PO₄, expected 32.6%). The DSC profile exhibited a melting endotherm at an onset of 229.3° C., peak maximum of 236.1° C., and enthalpy of fusion of 121.7 J/g. The melting endotherm was preceded by a broad desolvation endotherm and followed by decomposition (FIG. 9C). The TGA profile shows a 4.01 wt % mass loss from 25 to 150° C. (FIG. 9D).

On the basis of Karl Fischer data, the diphosphate salt is likely a monohydrate. This salt can be easily synthesized and shows improved stability (thermally and chemically) over the other salts exemplified herein.

### Compound I Dibenzoate

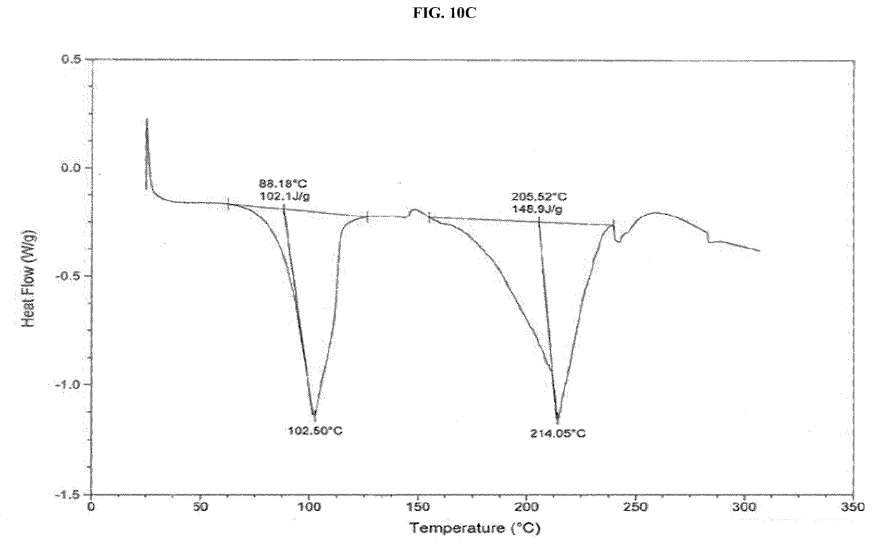

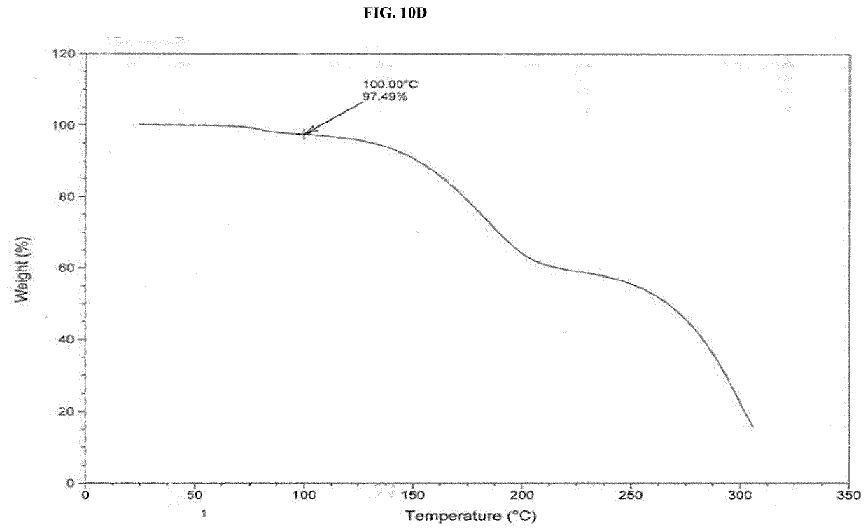

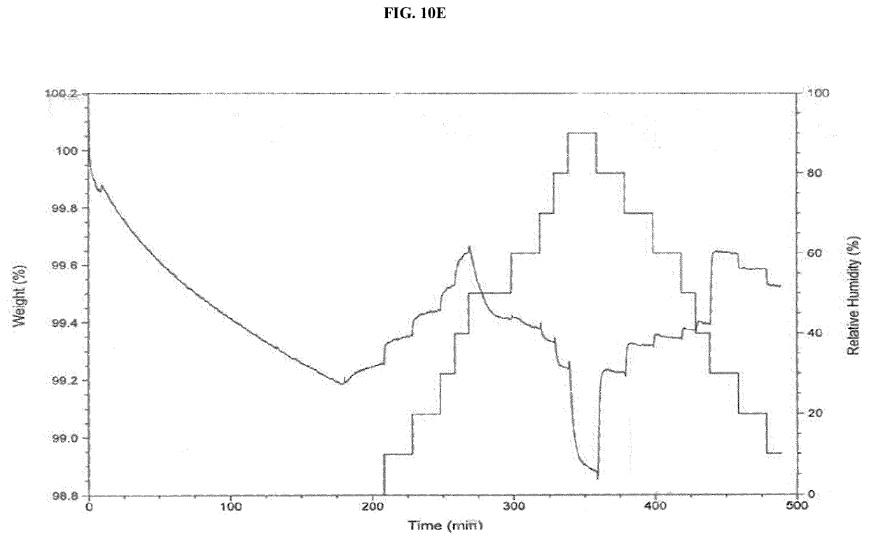

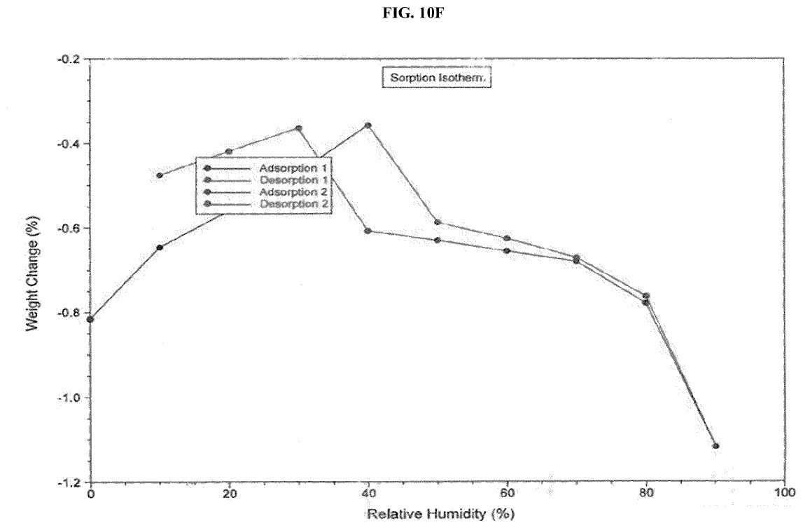

The XRD pattern of the dibenzoate salt indicated the batch was a crystalline solid as shown in FIG. 10A. This sample initially yielded a glassy material, but eventually crystallized after sitting in a fume hood for several weeks. The ¹H NMR spectrum of the dibenzoate salt is consistent with the protonated API and 2 molar equivalents of benzoic acid. No residual solvents apart from water were detected. The spectrum is shown in FIG. 10B. The DSC profile exhibited two broad endotherms with peak maxima at 103° C. and 214° C., followed by decomposition (FIG. 10C). The TGA profile shows a mass loss of 2.51 wt % from 25 to 150° C. (FIG. 10D). DVS data showed the salt was essentially non-hygroscopic with no significant moisture uptake. The kinetic and isotherm DVS plots are shown in FIGS. 10E-IOF. Further, preparation of a crystalline dibenzoate salt could not be reproduced under any conditions.

### Compound I Glycolate

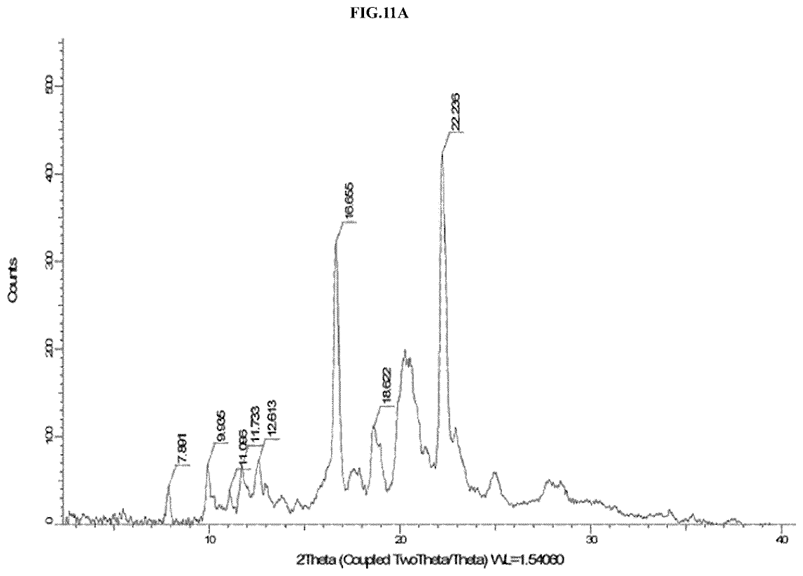

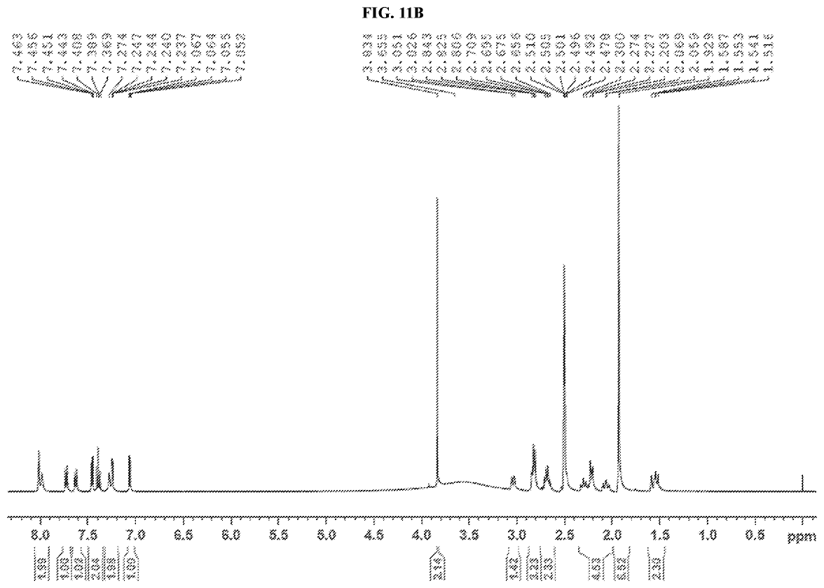

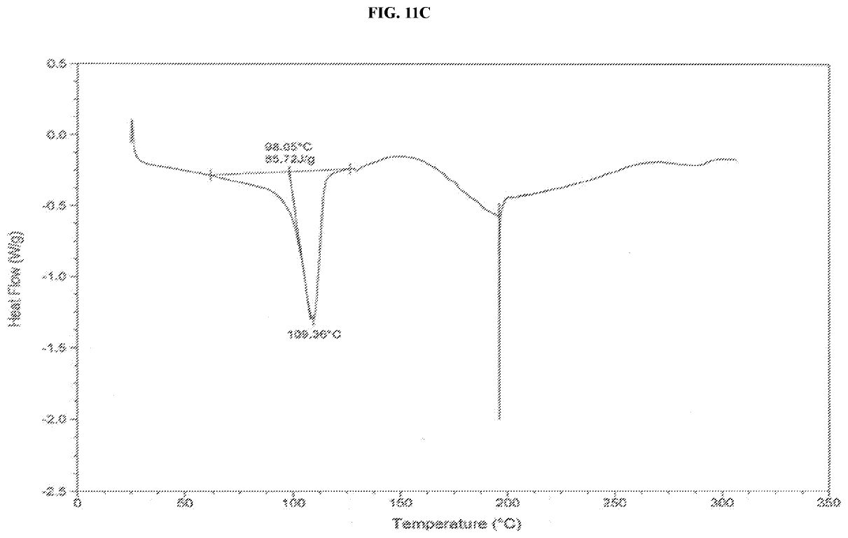

The XRD pattern of the glycolate salt indicated the batch was a partially crystalline solid as shown in FIG. 11A. The ¹H NMR spectrum of the glycolate salt is consistent with the protonated API and one molar equivalent of glycolic acid. The spectrum is shown in FIG. 11B. The DSC profile exhibited a broad desolvation endotherm with a peak maximum of 109° C., followed by decomposition (FIG. 11C). The TGA profile shows a mass loss of 5.15 wt % from 25 to 150° C. (FIG. 11D).

Based on thermal data, the glycolate salt was a hydrate which suffered from a lack of thermal stability.

### Summary of Salt Screen

A salt screening study was performed to identify suitable salt candidates of Compound I. The salt library explored thirty different salt forms as well as some potential co-formers. Characterization of the solids isolated during the primary salt screen facilitated the identification of five crystalline salt forms of Compound I (i.e., acetate, glycolate, dibenzoate, diphosphate and sulfate salts).

Four of the five salts suffered major setbacks which hindered the viability of the salts as the final API. The acetate and glycolate salts were hindered by poor thermal stability. The dibenzoate salt lacked appropriate reproducibility for isolation as a crystalline solid. Lastly, the sulfate salt was unfavorably hygroscopic for process conditions.

The diphosphate salt, however, exhibited good crystallinity and thermal properties, and has high aqueous solubility. The salt can be prepared and processed on a large scale and is stable to storage under ambient conditions.

Overall, Compound I was found to only form crystalline salts when water is incorporated into the crystal structure (i.e., hydrates). The diphosphate was found to be the preferred salt for this development of Compound I.

### Example 3: Polymorph Screen of the Compound I Diphosphate Salt

Based on the results of the primary salt screen described elsewhere herein, the diphosphate salt was selected for polymorph screening. The Compound I diphosphate salt was prepared at the 17 g scale by precipitation from aqueous acetone solution. The sample was subsequently dried under vacuum at 55° C. until the residual acetone content was <5000 ppm, and this material was used in the polymorph screening studies described herein without further purification.

The screen was performed using solvent recrystallizations using different conditions, as well as non-competitive slurry experiments, to manipulate the solid-state form of the test material. Samples generated during the study were characterized using differential scanning calorimetry (DSC), thermogravimetric analysis (TGA), powder X-ray diffraction (XRD), and dynamic vapor sorption (DVS). The polymorph screen revealed that Compound I diphosphate salt is monomorphic, existing as either a crystalline hydrate (Form A) or an amorphous solid.

### Materials and Methods

### Solvent Recrystallization

To perform the solvent-based portion of the polymorph screen, the test material was recrystallized using various solvents under approximately 78 different crystal growth conditions. The scale of the recrystallization experiments was approximately 1 to 20 mL. The method of changing the crystal growth conditions was by using single and binary arrays of solvent mixtures. The saturation temperature, growth temperature, and evaporation rate were also varied to create additional differences in crystal growth conditions.

Saturated solutions were prepared by agitating excess test material in contact with the various solvent systems at the saturation temperature. The mother liquor was separated from the residual solids by filtration if solids remained in the solution. The mother liquor was then heated above the saturation temperature to dissolve any remaining solids. The temperature of each solution was then adjusted to the growth temperature and a controlled nitrogen shear flow was introduced to begin solvent evaporation.

The recrystallization conditions for the three solvent based panels used during the study are summarized in Tables 14-16. Only Form A and the amorphous form were recovered during the recrystallization panels.

TABLE 14
Fast evaporation experiments from single
solvent systems at ambient temperature

| Solvent | Form |
|---|---|
| Ethanol | Amorphous |
| Isopropyl ether | * |
| Ethyl acetate | * |
| 2-butanol | * |
| Isopropyl acetate | * |
| Dioxane | * |
| DMF | Amorphous |
| Diethyl ether | * |
| MTBE | * |
| THF | * |
| Acetone | * |
| t-butanol | * |
| DMA | Amorphous |
| Chloroform | Amorphous |
| Nitromethane | * |
| 1-propanol | Amorphous |
| 2-methyl-tetrahydrofuran | * |
| Water | A |
| Toluene | * |
| 2-propanol | * |
| Methanol | Amorphous |
| 2-butanone | * |
| Heptane | * |
| Acetonitrile | * |

*Not enough solid recovered to determine form.

TABLE 15
Fast evaporation experiments from single solvent systems at 65° C.

| Solvent* | Form |
|---|---|
| Ethanol | Amorphous |
| Dioxane | * |
| 2-butanol | * |
| 1-propanol | Amorphous |
| THF | * |
| t-butanol | * |
| Ethyl acetate | * |
| DMF | A |
| DMA | A |
| Methanol | A |
| 2-butanone | * |
| Acetonitrile | * |
| 2-propanol | * |
| Water | A |

*10 drops of water were added to each sample;
**Not enough solid recovered to determine form.

TABLE 16
Fast evaporation experiments from binary
solvent systems at ambient temperature

| Solvent 1 | Solvent 2 | Form |
|---|---|---|
| Acetone | Water | A |
| Dioxane | Water | A |
| t-butanol | Water | Glass |
| Ethyl acetate | Water | A |
| Ethanol | Water | A |
| 1-propanol | Water | A |
| DMF | Water | Amorphous |
| THF | Water | A |
| 2-butanol | Water | Glass |
| 2-butanone | Water | Glass |
| Acetonitrile | Water | A |
| 2-propanol | Water | A |
| Methanol | Water | A |

### Crystallization by Cooling

Samples of Compound I diphosphate were dissolved in ~10 mL of organic solvent plus 10 drops of water, filtered, and placed in a freezer at −20° C. After several days, no crystallization was observed in any of the samples (Table 17).

TABLE 17
Crystallization by cooling experiments at −20° C.

| Solvent* | Form |
|---|---|
| Acetone | * |
| 2-butanol | * |
| DMA | * |
| Ethyl acetate | * |
| DMF | * |
| Ethanol | * |
| 1-propanol | * |
| THF | * |
| Methanol | * |
| Acetonitrile | * |
| 2-butanone | * |
| 2-propanol | * |

*10 drops of water were added to each sample;
**Not enough solid recovered to determine form.

### Non-Competitive Slurry Experiments

In addition to the solvent recrystallization experiments, non-competitive slurry experiments were performed to search for new solid-state forms. These experiments rely on solubility differences of different polymorphic form, if the compound exists in different polymorphic forms. As such, only polymorphs having a lower solubility (i.e., increased stability) than the original crystalline form can result from a non-competitive slurry experiment.

Essentially, when a solid is mixed with solvent to create slurry, a saturated solution is formed. The solution is saturated with respect to the polymorphic form dissolved. However, the solution is supersaturated with respect to any polymorphic form that is more stable (more stable forms have lower solubility) than the polymorphic form initially dissolved. Therefore, any of the more stable polymorphic forms can nucleate and precipitate from solution. In addition, non-competitive slurry experiments are often useful in identifying solvents that form solvates with the compound.

The slurry experiments described herein were performed by exposing excess supplied material (i.e., Form A) to solvents and agitating the resulting suspensions for one week at ambient temperature. The solids were filtered and analyzed by XRD to determine the resulting form(s). A summary ofnon-competitive slurry experiments is shown in Table 18.

Based on their X-ray scattering behavior, no change in form was observed in any of the slurry experiments.

TABLE 18
Non-competitive slurry experiments

| Solvent | Water Added | Appearance | Final Form |
|---|---|---|---|
| THF | 2 drops | White solid | A |
| t-butanol | 2 drops | White solid | A |
| 1-propanol | 2 drops | White solid | A |
| Ethanol | 2 drops | White solid | A |
| Dioxane | 2 drops | White solid | A |
| DMF | 2 drops | White paste | — |
| Methanol | 2 drops | White solid | A |
| Acetonitrile | 2 drops | White solid | A |
| 2-propanol | 2 drops | White solid | A |
| 2-butanol | 2 drops | White solid | A |
| Acetone | 2 drops | White solid | A |
| Acetone | 10% | White solid | A |
| Acetone | 20% | White solid | A |
| Acetone | 30% | White solid | A |
| Acetone | 50% | —* | —* |

*Not enough solid recovered to determine form.

### Static Vapor Sorption Studies

Static vapor sorption studies were done in hermetic humidity chambers using saturated salts, or environmental humidity chambers to control the temperature and humidity. Data collected during dynamic vapor sorption studies often are not at thermodynamic equilibrium. To gain a better understanding of the water uptake and the critical humidity level of the API, samples of the starting material (i.e., Form A) were monitored in static humidity chambers to allow the sample to equilibrate with atmospheric moisture.

In these studies, water vapor sorption was determined gravimetrically by storing samples in open aluminum dishes. Compound I diphosphate salt lot was used for this study. Samples were stored at 7 different humidity levels at ambient temperature (Table 19). Approximately 50 mg of Form A was staged at each humidity condition. The samples were weighed before placing them in the chamber and then re-weighed after 3 weeks. After re-weighing, the samples were immediately analyzed by XRD to determine whether a change in form had occurred.

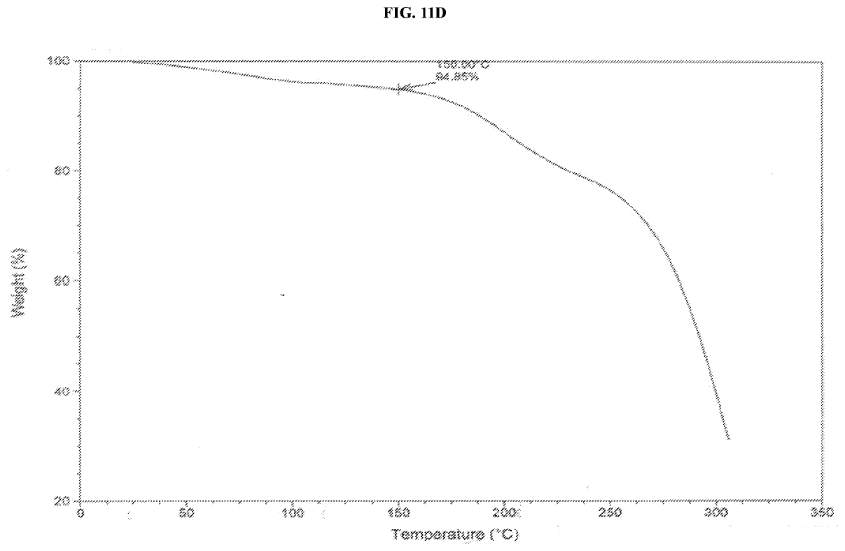

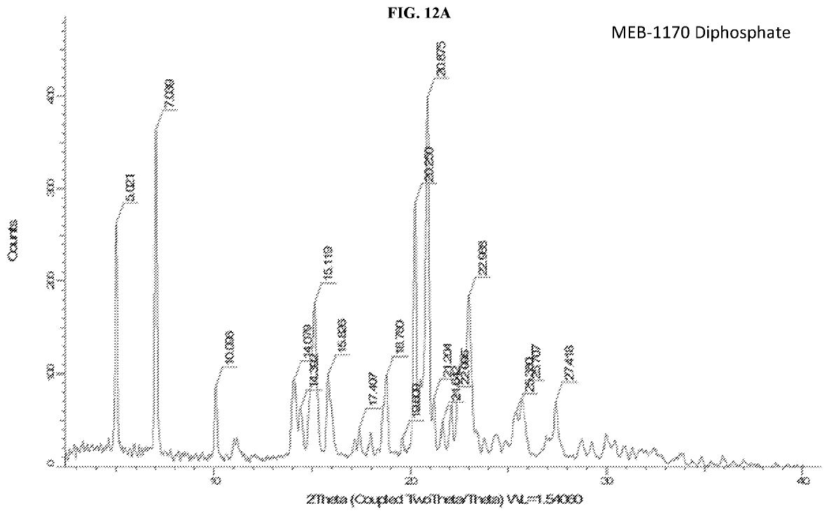

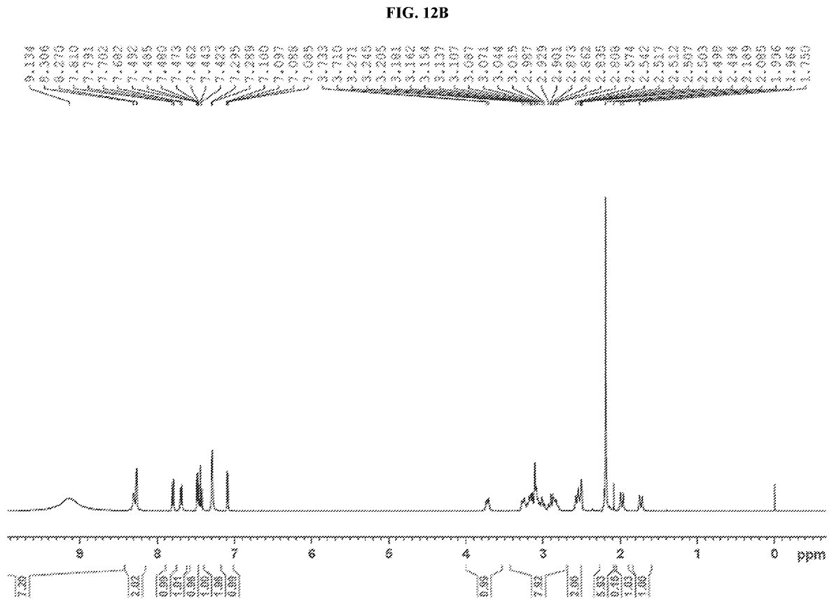

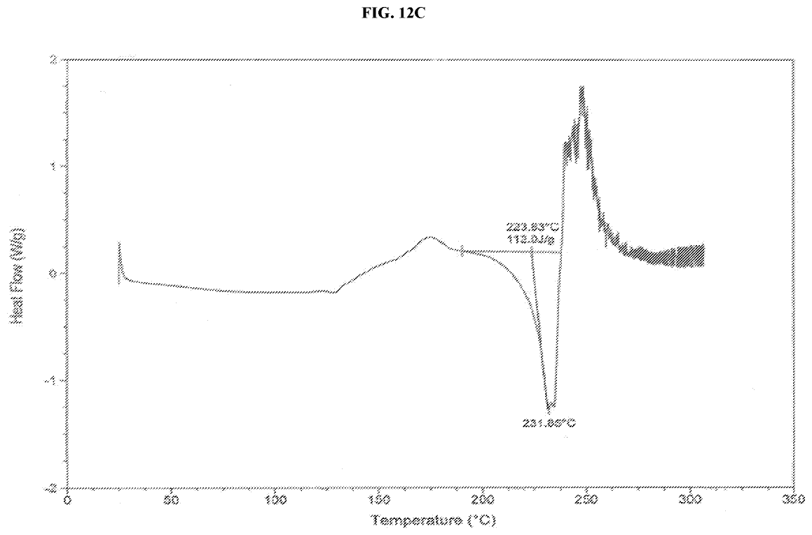

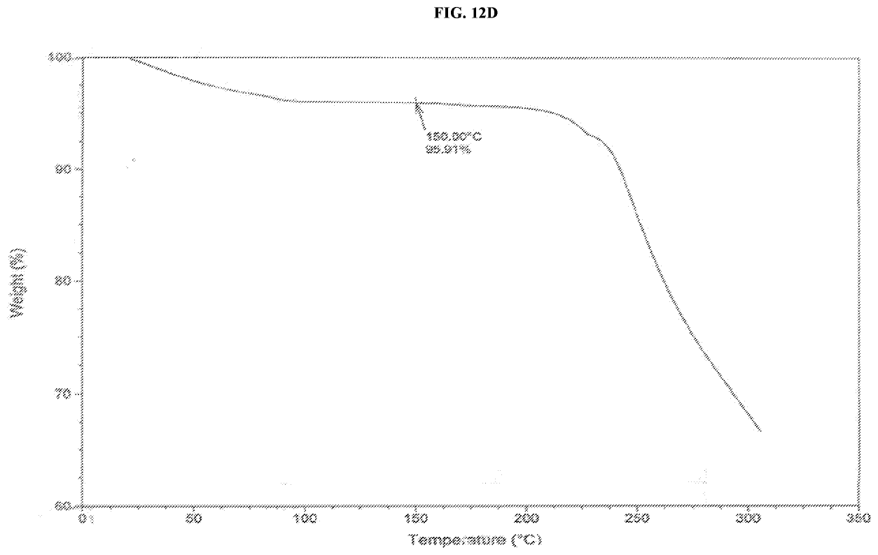

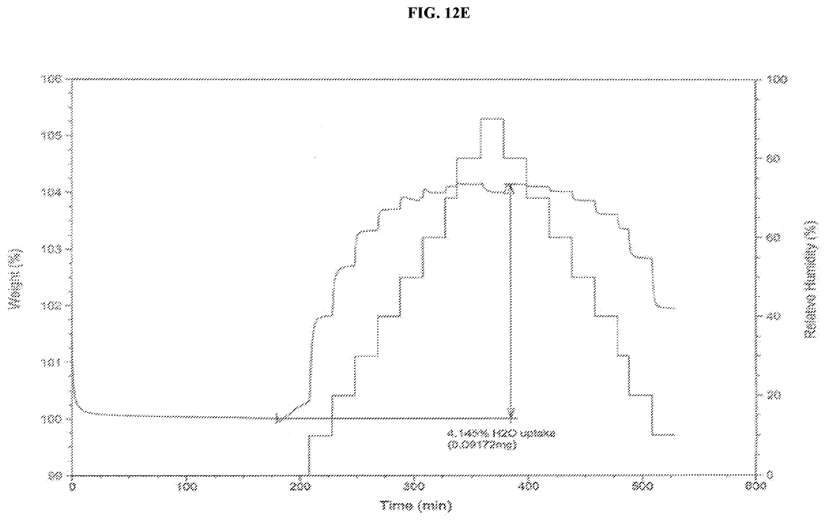

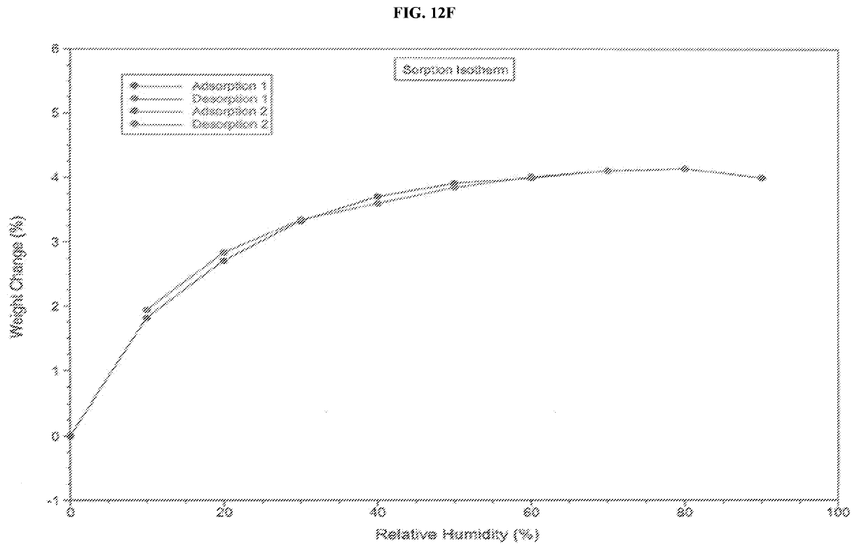

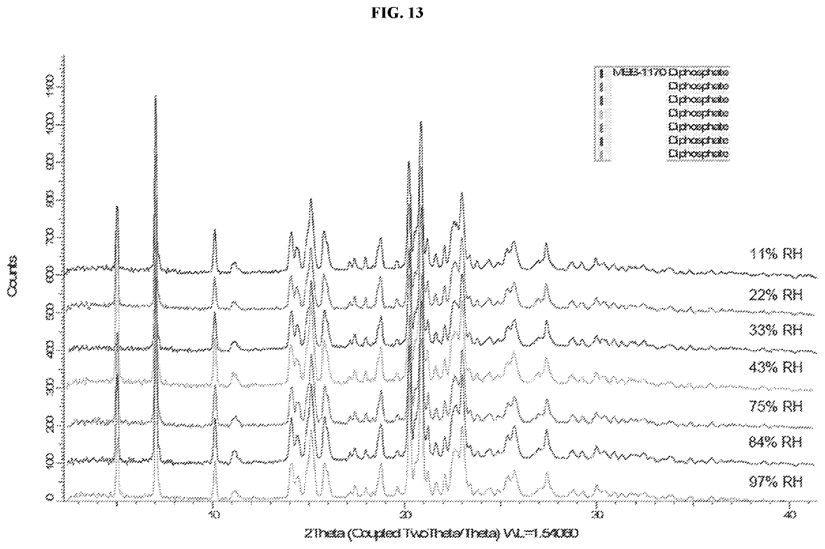

The sample stored at 11% RH was an outlier and was therefore omitted from the final isotherm plot. The static isotherm plot is roughly consistent with the DVS isotherm plot (FIG. 12F) with a large moisture uptake at around 40% RH and a maximum uptake between 4 and 5 weight percent. The solid state form of the salt did not change at any humidity level. The overlay of XRD patterns for the static vapor sorption experiments is shown in FIG. 13.

TABLE 19
Static vapor sorption experiments

| Relative | Wt % | Est. H₂O | |
|---|---|---|---|
| Humidity (%) | Change | Content (%) | Form |
| 11 | −0.2 | 3.1 | A |
| 22 | −2.5 | 0.8 | A |
| 33 | −1.5 | 1.8 | A |
| 43 | +0.6 | 3.9 | A |
| 75 | +0.8 | 4.1 | A |
| 84 | +1.1 | 4.4 | A |
| 97 | +1.5 | 4.8 | A |

### X-Ray Analysis of Screening Samples

Solids generated from the solvent based recrystallization panels and otherwise provided samples were analyzed by powder XRD. To mitigate preferred grain effects, a two dimensional detection system was used to collect all the XRD screening data. The two dimensional detector integrates along the concentric Debye cones which helps reduce pattern variation. If bright spots appear in the conical rings, it indicates strong preferred grain effects that can lead to considerable variability in the observed diffraction patterns including changes in peak intensities. Some samples of Compound I diphosphate exhibited preferred grain effects based on the appearance of the scattering behavior.

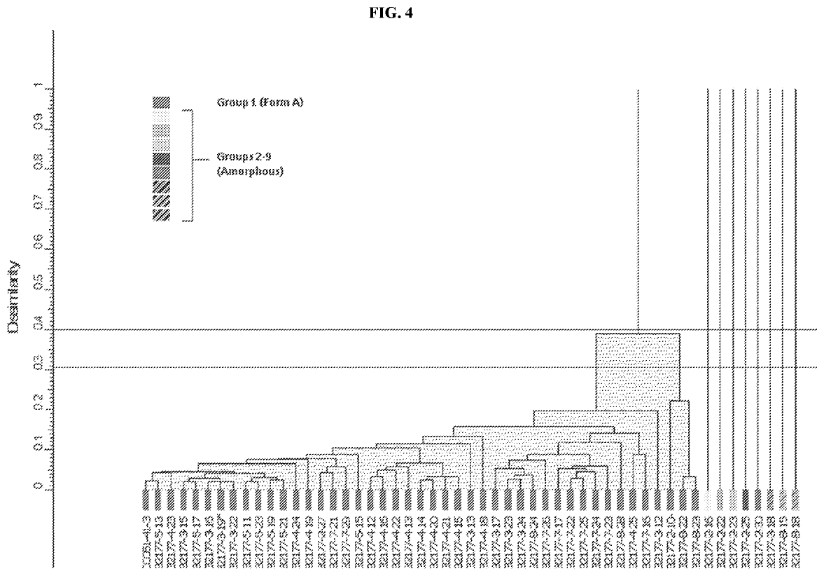

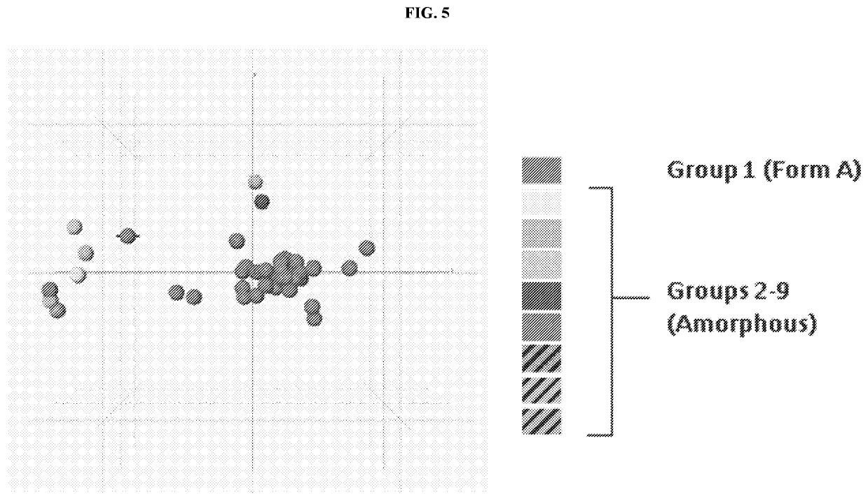

The results of this analysis revealed Compound I diphosphate exists as one primary hydrate form (i.e., Form A) as well as an amorphous form. The XRD data collected during the study was evaluated using a full profile chemometric treatment to determine if the crystalline form of the samples had changed during the recrystallization and other manipulations performed during the study. The analysis entailed cluster analysis and multivariate statistics to group together any patterns that were determined to be statistically the same. The results of this analysis are summarized in the dendrogram and cluster plots disclosed herein (FIGS. 4-5).

In the dendrogram, each pair of diffraction patterns is connected via a single node, which corresponds to a dissimilarity value (between 0 and 1) on the y-axis. The higher the connecting node is, the more dissimilar the two patterns are. A threshold dissimilarity value (~0.4 in this case) is used to group similar patterns into clusters, which should correspond to different solid state forms. The cluster plot is a 3D representation of the same analysis.

The chemometric analysis of the diffraction data categorized the samples into 9 different groups labeled 1-9. Practically speaking, however, there are only 2 groups: one for Form A and another for the amorphous samples. A summary showing the number of members in each group is shown in Table 20.

TABLE 20
Approximate number of members in each group

| Group | Designated Form | Members |
|---|---|---|
| 1 | A | 45 |
| 2-9 | Amorphous | 8 |

### Characterization of Supplied Material (i.e., Compound I Diphosphate Salt)

The starting material was characterized by XRD, NMR, TGA, and DVS. X-ray powder diffraction was used to examine the supplied lot to determine if it was crystalline. FIG. 12A shows the XRD pattern of this material. The starting material was confirmed crystalline and identified as Form A. The ¹H NMR spectrum was found consistent with the expected structure of Compound I diphosphate (FIG. 12B).

The thermal characteristics of the staring material was determined by DSC and TGA. The DSC thermogram exhibited a melting endotherm with an onset of 223.8° C. and peak maximum at 231.9° C. (FIG. 12C). The heat of fusion was 112.0 J/g. A broad desolvation endotherm precedes the melting endotherm. The TGA thermogram showed a mass loss of 4.09 wt % from 25° C. to 150° C. (FIG. 12D).

### Polymorph Screening

The purpose of the polymorph screen was to discover as many crystalline forms of the compound as possible. The screen was performed using solvent-based recrystallization, followed by X-ray diffraction analysis of the solids. Suspension slurry experiments were also employed to search for additional solid-state forms.

To perform the solvent-based portion of the polymorph screen, the test material was recrystallized using various solvents under different crystal growth conditions. The scale of the recrystallization experiments was approximately 10-50 mg. The techniques employed are listed below.

### Characterization of Polymorph Forms

After classifying the data into different forms based on diffraction behavior, each form was studied to determine if other properties of the forms could be differentiated. The characterization of each form began by comparing the diffraction data representative of each form with that from the other forms. This was generally followed by NMR, DSC, and TGA.

### Form A

Form A was obtained in most crystallization and slurry experiments which contained water. The characteristic diffraction behavior of this form is shown in FIG. 12A. The ¹H NMR spectrum of Form A is consistent with the expected structure of Compound I diphosphate (FIG. 12B).

The DSC thermogram exhibited a melting endotherm with an onset of 223.8° C. and peak maximum at 231.9° C. The heat of fusion was 112.0 J/g. A broad desolvation endotherm precedes the melting endotherm (FIG. 12C). The TGA thermogram for Form A showed a mass loss of 4.09 wt % from 25° C. to 150° C. (FIG. 12D). A DVS scan of Form A showed it was moderately hygroscopic, with a maximum moisture uptake of 4.15 wt % at 90% RH (FIG. 12E). No hysteresis was observed in the isotherm plot, shown in FIG. 12F. The moisture content of Form A at ambient conditions, as determined by coulometric Karl Fischer titration, was 3.29 wt % (3.0 wt % expected for a monohydrate).

Overall, Form A is the only known crystalline form of Compound I diphosphate. Based on its Karl Fischer moisture content, Form A is a monohydrate.

### Amorphous Form

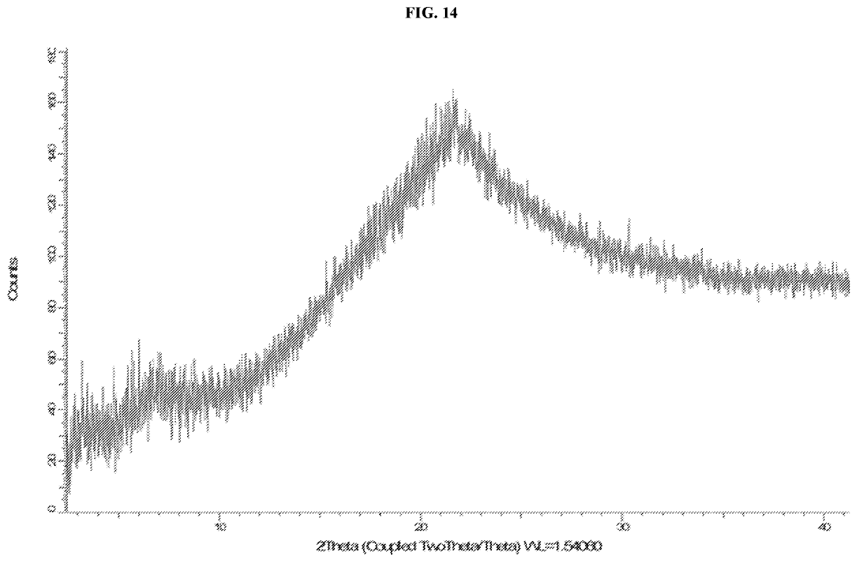

The amorphous form was obtained by crystallization in anhydrous solvents. The characteristic diffraction behavior of this form is shown in FIG. 14. No other characterization data was collected for the amorphous form.

### Summary of Polymorph Screen

A total of 100 Compound I diphosphate samples from the polymorph screen crystallization panels were produced during the course of the study, as summarized in Table 21.

TABLE 21
Summary of polymorph screening for the free acid of Compound I

| | No. of | Form | Amorphous | No solid |
|---|---|---|---|---|
| Panel | Experiments | A | Form | recovered |
| Fast evaporation, single | 24 | 1 | 6 | 17 |
| solvent, ambient | | | | |
| Fast evaporation, single | 14 | 4 | 2 | 8 |
| solvent, 65° C. | | | | |
| Cooled to −20° C., | 12 | 0 | 0 | 12 |
| single solvent | | | | |
| Fast evaporation, | 13 | 9 | 1 | 3 |
| binary solvent | | | | |
| Anti-solvent addition | 15 | 9 | 0 | 6 |
| Non-competitive slurries | 15 | 13 | 0 | 2 |
| Static humidity | 7 | 7 | 0 | 0 |
| Total | 100 | 43 | 9 | 48 |
| % of Total | 100% | 43% | 9% | 48% |

The raw diffraction data generated from the polymorph screening experiments (i.e., solvent recrystallization and non-competitive slurries) were categorized into two forms. Samples of these different forms were used to perform additional experiments (e.g., DSC, TGA, and NMR, inter alia) to further characterize the forms. A brief description of the discovered solid forms is summarized in Table 22.

Compound I diphosphate was found to exist as one of two solid state forms: a crystalline hydrate (Form A) and an amorphous form. Form A is readily obtained by crystallization in the presence of water and is stable at ambient conditions. This hydrate retains its structure in both low and high humidity environments and can withstand heating up to ~70° C. The amorphous form may be obtained by crystallization from anhydrous solvents or by thermal desolvation of the hydrate. In certain embodiments, the preferred polymorph of Compound I diphosphate is Compound I diphosphate monohydrate (Form A):

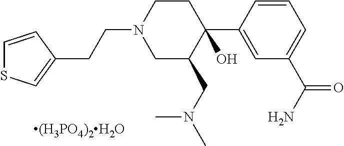 [SMILES: CN(C)C[C@H]1CN(CCc2ccsc2)CC[C@]1(O)c1cccc(C(N)=O)c1]

TABLE 22
Summary of different solid state forms

| Form | Description | Comments |
|---|---|---|
| A | Monohydrate | Crystalline hydrate obtained |
| | | using water |
| Amorphous | Amorphous | Non-crystalline form obtained |
| | | using anhydrous solvents |

### Example 4: Phase I, Double Blind, Placebo-Controlled Single and Multiple Oral, Ascending Dose Study to Assess Safety, Tolerability, Pharmacokinetics and Pharmacodynamics of Compound I Bisphosphate

A single ascending dose assessment with a food effect component was performed with Compound I diphosphate (Drug Substance or DS). The study comprised a single ascending dose (SAD), sequential cohort study, incorporating a food effect (FE) evaluation. Up to 40 subjects were to be studied in 5 cohorts (Cohorts A1 to A5), each cohort consisting of 8 subjects.

Subjects in Cohorts A1, A2, A4 and A5 participated in 1 treatment period only, residing at the CRU from Day −1 (the day before dosing) to Day 3 (48 hours post-dose). Subjects in Cohort A3 participated in 2 treatment periods (once in Fed state, once in Fasted state) separated by a minimum of 6 days. All subjects were to return for a poststudy on Day 5 (+2 days) after their final dose for a final safety assessment.

In each cohort, sentinel dosing occurred such that two subjects (one active and one placebo) were dosed at least 48 hours before the remaining subjects, where continuation to dose the remaining subjects was at the Investigator's discretion, in consultation with the sponsor.

In each of Cohorts A1 to A5, 6 subjects received Compound I bisphosphate and 2 subjects received placebo. All doses for Cohorts A1, A2, A4, and A5 were administered in the fasted state in the moming of Day 1.

### Food Effect:

Eight subjects in Cohort A3 participated in a 2-period treatment design in which they were assessed for the single-dose of drug substance in a Fasted condition and in a Fed condition. Subjects were to receive the same treatment (i.e., either drug substance or placebo) in both Period 1 and Period 2, and thus subjects would receive either two single doses of drug substance or two single doses of placebo during their participation in the study. Fasting state assessments occurred in Period 1 and Fed State assessments in Period 2. It was planned that an approximate 7-day washout would be needed between the Fast and Fed dosing periods. Note that PD assessments were not performed for the food effect Cohort A3.

Planned dose levels are described in Table 23. For each Part A Cohort, sentinel dosing occurred such that two subjects (one active and one placebo) were dosed at least 48 hours before the remaining subjects, where continuation to dose the remaining subjects was at the Investigator's discretion, in consultation with the sponsor. Note that the blinding for these subjects remained throughout the treatment period.

TABLE 23
Study Part A Planned and Actual Dose Levels - Single Doses

| | | | Total | | | |
|---|---|---|---|---|---|---|
| | Planned | | Amount | | | |
| | and Actual | | (mg) Drug | | Number of Subjects | |
| | Dose of Drug | | Substance | | | Drug |
| Cohort | Substance | | (or Placebo) | | Placebo | Substance |
| A1 | 20 | mg Fasting | 20 | mg | 2 | 6 |
| A2 | 60 | mg Fasting | 60 | mg | 2 | 6 |
| A3 | Period 1: 120 | | 240 | mg | 2 | 6 |
| (2-period | mg Fasting | | | | | |
| FE) | Period 2: 120 | | | | | |
| | mg Fed | | | | | |
| A4 | 200 | mg Fasting | 200 | mg | 2 | 6 |
| A5 | 400 | mg Fasting | 400 | mg | 2 | 6 |

FE = Food Effect

For all cohorts EXCEPT the food-effect cohort, morning doses are administered after completing an overnight fast of at least 8 hours. Standard breakfast is available approximately 90 minutes following morning dosing.

For the food-effect cohort, dosing is administered in a fasted state and then in a fed state.

### Diagnosis and Main Criteria for Inclusion:

This study was designed to enroll healthy adult subjects, and the eligibility criteria reflected that design element.

Key eligibility included:
1. Healthy males and females as determined by medical history, physical examination, laboratory, and ECG findings
2. Age—18 to 55 years inclusive
3. BMI—18 to 32 kg/m² inclusive
4. Ethnic origin—Any
5. Smoking status—Current Non-smokers
6. Negative Covid antigen or PCR test
7. Ability to tolerate cold pressor test

### Test Product, Dose and Mode of Administration:

Compound I is a signaling pathway-selective μ-opioid agonist. Compound I bisphosphate (Drug Substance) is a crystalline white to off-white solid and was encapsulated in size 2 capsules for oral dosing. Dosage strengths were 20 mg and 40 mg capsules.

### Duration of Treatment:

Each subject participated in 1 treatment period only, residing at the CRU from Day −1 (the day before dosing) to Day 3 (48 hours post-dose), except for Cohort A3 (food effect assessment), where each subject participated in 2 treatment periods, separated by a minimum of 6 days. Subjects returned for a poststudy visit on Day 5 (+2 days) for a final safety assessment.

This was a single-ascending dose study, and thus the duration of treatment with study medication was one day (reflecting one dose per subject). However, for the food effect portion of the study, subjects were given a single dose in a fasting state, and then again in a fed state, and thus had two doses with a washout period in-between.

### Reference Therapy, Dose and Mode of Administration, Batch Number:

Placebo capsules mimicking the active capsules in appearance were utilized for the purpose of blinding and were also orally dosed.

### Criteria for Evaluation:

### Pharmacokinetics:

Single Dose PK: Plasma Compound I concentrations and those of the primary metabolite Compound II were be measured after a single oral dose of drug substance at pre-dose and at 0.5, 1, 1.5, 2, 2.5, 3, 3.5, 4, 6, 8, 12, 16, 24, 36, and 48 hours post-dose

Food Effect: Within the single dose portion of the study, the third cohort of subjects (A3) completed a second treatment period in which plasma Compound I and Compound II concentrations were measured at pre-dose and 0.5, 1, 1.5, 2, 2.5, 3, 3.5, 4, 6, 8, 12, 16, 24, 36, and 48 hours post-dose, following receipt of a high fat meal 30 minutes prior to dosing. Note that PD assessments were not performed for the Food Effect cohort.

### Safety:

The safety assessments include clinical laboratory assessments (chemistry, hematology, urine), ECGs, physical examinations, vital signs, and adverse events.

### Pharmacodynamics:

Pharmacodynamic (PD) responses were assessed via pupillometry, capnography, oximetry, and cold pressor testing. Pupillometry, capnography, and oximetry assessments occurred pre dose (Day −1) and at hours 3, 6, and 9 post dose. For cold pressor, assessments occurred at Screening (to confirm subject ability to perform the test), and then pre dose (Day −1) and then at 4.5 and 8.5 hours post dose.

### Statistical Methods:

### Sample Size

This was a first-in-human study, in which we assessed the single ascending dose effects of drug substance on tolerability, safety, PK, and PD endpoints. A sample size of eight subjects per cohort, 6 treated with drug substance and 2 treated with matching placebo, is consistent with other similar studies at this stage of clinical development. The sample size allowed for a careful escalation from lower to higher doses and adequately characterized the rate and extent of drug absorption as measured by selected PK parameters.

### Pharmacokinetics

The actual blood sampling dates and times relative to dosing time were listed by subject and nominal sampling time, with time deviation calculated, for all subjects with available plasma concentration data. Individual (for each subject) and mean Compound I and Compound II concentrations over time (for each cohort) were displayed graphically in linear and semi-logarithmic plots. The actual collection time was used for individual plasma Compound I and Compound II concentration curves and the nominal time was used for the plots of mean plasma Compound I and Compound II concentration curves.

For PK concentration data, the number of non-missing values, number of below limit of quantification (BLQ) values, arithmetic mean, standard deviation, median, minimum, maximum, coefficient of variation (CV %), geometric mean and geometric coefficient of variation (geo CV %) values was presented.

For PK parameter data, the number of non-missing values, arithmetic mean, standard deviation, median, minimum, and maximum values were presented.

### Food Effect

For Part A Cohort A3, assessment of the food-effect was performed for the following PK parameters:
AUC₀₋ₗₐₛₜ
Cₘₐₓ
AUC_{0-inf}

Due to the small sample size of this study, it was concluded that the rate and extent of absorption was no different between the test and reference groups if the point estimate for AUC_{0-inf} (or AUC₀₋ₗₐₛₜ, if AUC0-inf could not be reliably estimated for each fed/fasted condition in all subjects) and the Cmax ratios fell within the range of 80 to 125%.

### Safety

Safety data included adverse events, clinical laboratory, vital signs, ECGs, and physical examinations. TEAEs were defined as adverse events that occurred following the first administration of study treatment. All TEAEs were coded using MedDRA. Subjects were counted only once for each system organ class (SOC) and preferred term (PT) level (categorical descriptive analysis).

Vital sign, clinical laboratory, and ECG measurements were presented using summary statistics for the results at baseline and each scheduled post-baseline visit for each of the parameters. In addition, summaries were presented for the change from baseline values at each scheduled post-baseline visit (continuous descriptive analysis). Graphics were used to allow for visual inspection of dose-response trends over time.

### Demographic and Other Baseline Characteristics:

Demographic and other baseline characteristics are summarized in Table 24. Overall, 62.5% of subjects were female, the majority were White (70.0%), with 42.5% Hispanic or Latino, the median age was 39.5 years (range of 21 to 56 years), and subjects had a median BMI of 26.45 kg/m² (range 19.5 to 31.5). The treatment groups were similar with respect to these demographic and baseline characteristics, with no clinically meaningful differences observed between the treatment groups. Note that Table 24 displays the placebo subjects in two columns, one for the primary SAD dosing escalation cohorts (n=8 subjects), and one for the food-effect cohort [indicated as Placebo (Fed/Fast), n=2 subjects]. This sums to n=10 placebo subjects in total.

TABLE 24
Demographic and Other Baseline Characteristics (Safety Population)

| | | | | Placebo | 120 mg | | | |
|---|---|---|---|---|---|---|---|---|
| | Placebo | 20 mg | 60 mg | (Fed/Fast) | (Fed/Fast) | 200 mg | 400 mg | Total |
| Category | (N = 8) | (N = 6) | (N = 6) | (N = 2) | (N = 6) | (N = 6) | (N = 6) | (N = 40) |
| Age (years), n | | | | | | | | |
| Mean | 41.1 | 37.7 | 39.3 | 44.0 | 29.5 | 43.8 | 36.0 | 38.4 |
| SD | 10.12 | 12.94 | 13.85 | 5.66 | 7.40 | 7.52 | 11.38 | 10.82 |
| Median | 44.0 | 35.5 | 36.0 | 44.0 | 27.5 | 45.5 | 35.5 | 39.5 |
| Minimum | 27 | 25 | 24 | 40 | 23 | 32 | 21 | 21 |
| Maximum | 56 | 54 | 56 | 48 | 43 | 51 | 52 | 56 |
| n | 8 | 6 | 6 | 2 | 6 | 6 | 6 | 40 |
| Sex, n (%) | | | | | | | | |
| Female | 5 (62.5%) | 5 (83.3%) | 3 (50.0%) | 2 (100%) | 3 (50.0%) | 3 (50.0%) | 4 (66.7%) | 25 (62.5%) |
| Male | 3 (37.5%) | 1 (16.7%) | 3 (50.0%) | — | 3 (50.0%) | 3 (50.0%) | 2 (33.3%) | 15 (37.5%) |
| Ethnicity | | | | | | | | |
| Not Hispanic | 3 (37.5%) | 3 (50.0%) | 6 (100%) | 1 (50.0%) | 4 (66.7%) | 4 (66.7%) | 2 (33.3%) | 23 (57.5%) |
| or Latino | | | | | | | | |
| Hispanic | 5 (62.5%) | 3 (50.0%) | — | 1 (50.0%) | 2 (33.3%) | 2 (33.3%) | 4 (66.7%) | 17 (42.5%) |
| or Latino | | | | | | | | |
| Race | | | | | | | | |
| White | 7 (87.5%) | 5 (83.3%) | 2 (33.3%) | — | 4 (66.7%) | 5 (83.3%) | 5 (83.3%) | 28 (70.0%) |
| Black or | 1 (12.5%) | 1 (16.7%) | 3 (50.0%) | 1 (50.0%) | 1 (16.7%) | 1 (16.7%) | 1 (16.7%) | 9 (22.5%) |
| African | | | | | | | | |
| American | | | | | | | | |
| Asian | — | — | 1 (16.7%) | — | 1 (16.7%) | — | — | 2 (5.0%) |
| Native | — | — | — | 1 (50.0%) | — | — | — | 1 (2.5%) |
| Hawaiian | | | | | | | | |
| or Other | | | | | | | | |
| Pacific | | | | | | | | |
| Islander | | | | | | | | |
| Baseline | | | | | | | | |
| body mass | | | | | | | | |
| index | | | | | | | | |
| (kg/m²), n | | | | | | | | |
| Mean | 26.04 | 27.48 | 28.60 | 31.05 | 24.80 | 24.27 | 25.47 | 26.35 |
| SD | 3.734 | 1.246 | 3.401 | 0.636 | 3.789 | 2.818 | 2.517 | 3.340 |
| Median | 26.15 | 27.80 | 29.05 | 31.05 | 24.70 | 23.85 | 25.65 | 26.45 |
| Minimum | 19.5 | 25.3 | 22.1 | 30.6 | 20.0 | 20.8 | 22.0 | 19.5 |
| Maximum | 30.9 | 28.6 | 31.4 | 31.5 | 31.2 | 28.5 | 28.4 | 31.5 |
| n | 8 | 6 | 6 | 2 | 6 | 6 | 6 | 40 |

Abbreviations: n = number, SD = standard deviation. Percentages based on the Safety Population.

The target population for enrollment in this study was healthy normal adult subjects, and based on the medical histories reported, the actual enrolled subject sample was healthy and normal.

All subjects were negative for alcohol test, drugs of abuse, and cotinine tests. No subjects tested positive for Covid-19 during the study. Females of child-bearing potential were all negative for pregnancy tests.

### Pharmacokinetic Results:

### Analysis of Pharmnacokineties

PK analyses were performed for the subset of patients in the study accordingly.

### Plasma Concentrations

### Compound I

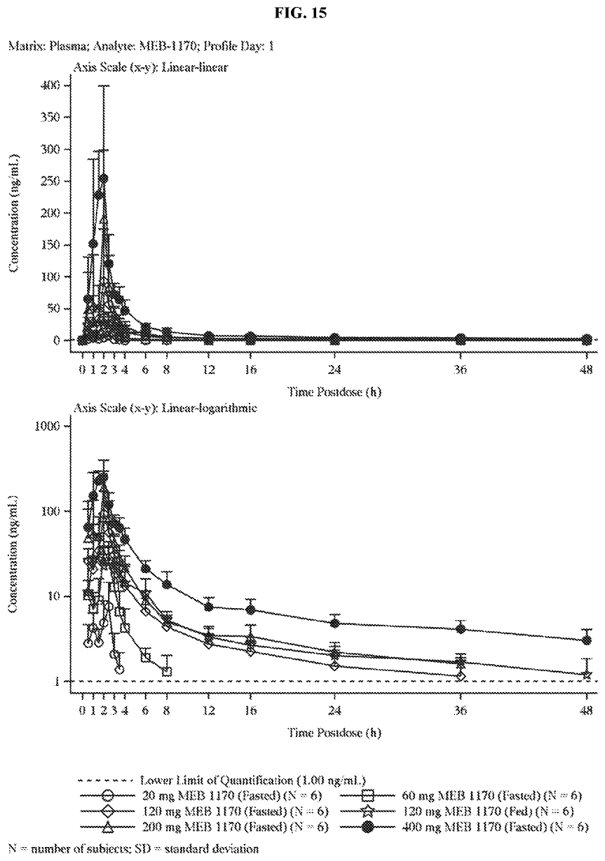

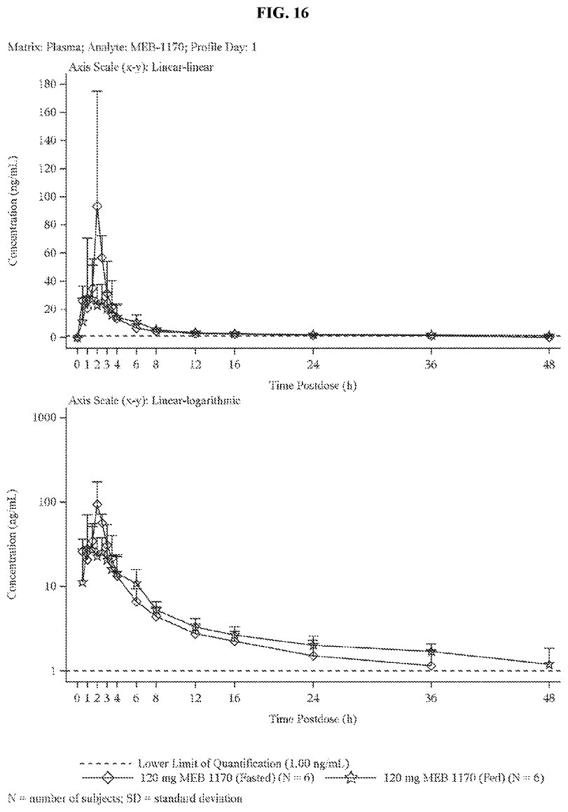

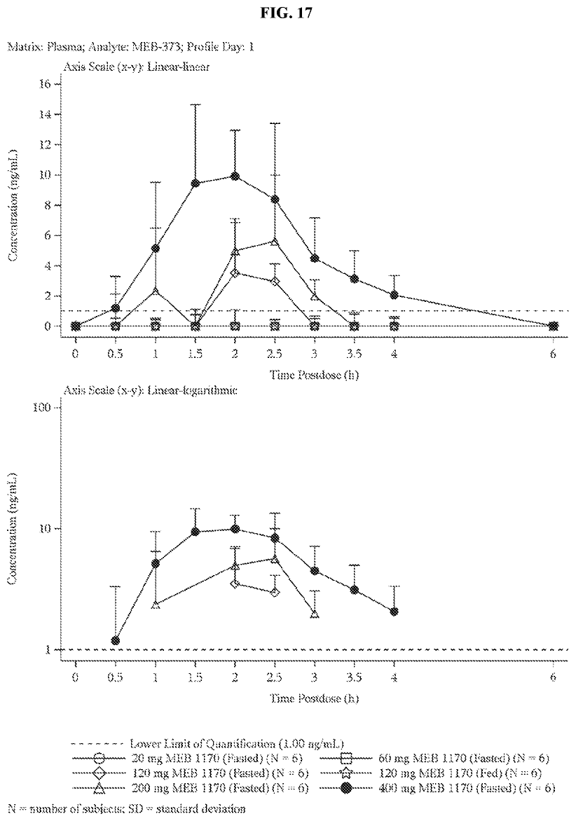

The Compound I concentration-time profiles on a linear and logarithmic scale for all groups are provided in FIG. 15 and the 120 mg dose administered under fasted and fed conditions is presented in FIG. 16.

Plasma concentrations of Compound I rose rapidly in subjects who received a single oral dose of Drug Substance on Day 1, and individual maximum concentrations were observed from 0.5 to 6.0 hours post-dose across the dose range. Compound I median tₘₐₓ values were 1.5 to 2.5 hours across dose levels under fasted conditions. After the peak, Compound I mean concentrations declined in a multi-exponential fashion and were measurable through 3.5, 8, 36, and 36 hours post-dose at doses of 20, 60, 120 (fasted), and 200 mg, respectively, and through the end of the sampling interval (48 hours post-dose) for subjects who received 120 mg (fed) and 400 mg.

### Compound II

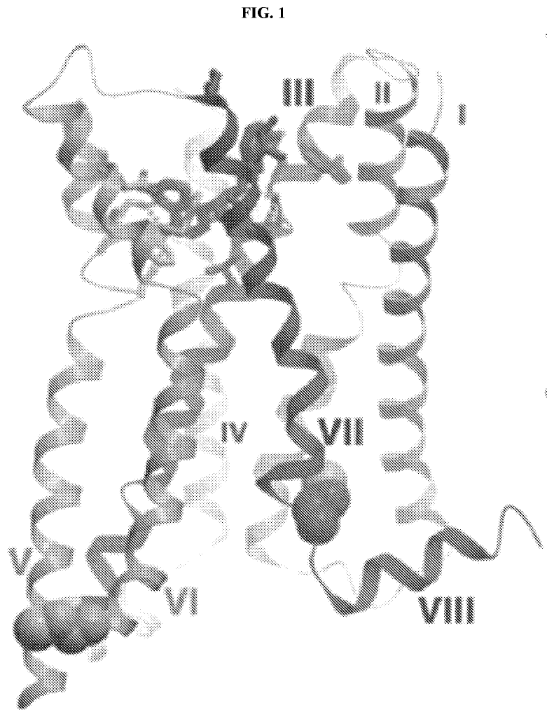

The Compound II concentration-time profiles on a linear and logarithmic scale are provided in FIG. 1.

Compound II concentrations in samples from most subjects at doses of 20, 60, and 120 (fed) mg were below the limit of assay quantitation across time points; therefore, only limited data were available for Compound II in these dose groups.

Mean plasma concentrations of Compound II rose in parallel with Compound I in subjects who received a single oral dose of Compound I on Day 1, with the maximum mean concentrations of 3.53, 5.63 and 9.92 ng/mL, observed at 2.0, 2.5, and 2.0 hours at the doses of 120 (fasted), 200 and 400 mg, respectively. Compound II mean concentrations declined after the peak and were measurable through 2.5, 3, and 4 hours post-dose at doses of 120 (fasted), 200, and 400 mg, respectively.

### Pharmacokinetic Parameters:

### Compound I:

PK parameters are summarized in Table 25.

TABLE 25
Summary of Pharmacokinetic Parameters of Compound I (Drug Substance
or DS) Following a Single Dose Oral Administration

| | | 20 mg | 60 mg | 120 mg | 120 mg | 200 mg | 400 mg |
|---|---|---|---|---|---|---|---|
| | | DS | DS | DS | DS | DS | DS |
| | | (Fasted) | (Fasted) | (Fasted) | (Fed) | (Fasted) | (Fasted) |
| Parameter | Statistic | (N = 6) | (N = 6) | (N = 6) | (N = 6) | (N = 6) | (N = 6) |
| Cₘₐₓ | Mean | 9.69 | 51.7 | 98.5 | 45.2 | 220 | 318 |
| (ng/mL) | SD | 6.25 | 30.6 | 78.6 | 35.2 | 80.0 | 113 |
| Tₘₐₓ (h) | Median | 2.50 | 2.25 | 2.28 | 2.58 | 2.00 | 1.50 |
| | Min | 0.500 | 2.00 | 2.00 | 1.00 | 1.00 | 1.00 |
| | Max | 2.50 | 3.02 | 3.00 | 6.02 | 2.02 | 2.02 |
| Tₗₐₛₜ (h) | Median | 4.00 | 12.0 | 36.0 | 48.0 | 48.0 | 48.0 |
| | Min | 3.50 | 6.00 | 16.0 | 36.0 | 36.0 | 48.0 |
| | Max | 4.03 | 24.0 | 48.0 | 48.0 | 48.1 | 48.0 |
| AUC₀₋ₜ | Mean | 13.9 | 67.1 | 231 | 204 | 381 | 794 |
| (h*ng/mL) | SD | 5.08 | 25.5 | 76.5 | 51.3 | 59.3 | 224 |
| AUC₀₋ₗₐₛₜ | Mean | 16.5 | 71.6 | 211 | 166 | 346 | 698 |
| (h*ng/mL) | SD | 5.10 | 27.4 | 64.7 | 40.0 | 62.1 | 199 |
| AUC_{0-inf} | Mean | 18.4 | 79.8 | 245 | NC | 446 | 832 |
| (h*ng/mL) | SD | 4.26 | 42.6 | 83.3 | NC | 47.1 | 173 |
| t_{1/2} (h) | Mean | 1.05 | 7.89 | 31.4 | 32.2 | 29.4 | 32.1 |
| | SD | 0.456 | 5.71 | 18.1 | 5.78 | 9.13 | 10.4 |
| CL/F (L/h) | Mean | 1130 | 937 | 541 | NC | 452 | 500 |
| | SD | 270 | 505 | 226 | NC | 51.1 | 116 |
| V_{z}/F (L) | Mean | 1380 | 6790 | 12300 | NC | 18100 | 20400 |
| | SD | 459 | 3020 | 4280 | NC | 6970 | 6570 |

AUC_{0-inf} = area under the plasma concentration-time curve from time 0 extrapolated to infinite time; AUC₀₋ₗₐₛₜ = area under the plasma concentration-time curve from zero to 24 hours postdose; AUC₀₋ₜ = area under the plasma concentration-time curve from zero to the last quantifiable concentration; CL/F: apparent clearance; Cₘₐₓ = maximum observed plasma concentration; Max = maximum; Min = minimum; NC = not calculated (n ≤ 2); SD = standard deviation; t_{1/2} = terminal plasma half-life; Tₘₐₓ = time to achieve maximum plasma concentration; Tₗₐₛₜ = time of last measurable concentration; V_{z}/F = apparent volume of distribution during the terminal phase.
Note:
Tₘₐₓ and Tₗₐₛₜ values are presented as median and other parameters are presented as arithmetic mean and SD.

Following a single oral administration of Drug Substance in subjects in the fasted state, plasma terminal t_{1/2} values for Compound I increased with increasing dose from 20 to 60 mg, with mean values of 1.05 and 7.89 hours, respectively, then increased above 60 mg and were similar over the 120 to 400 mg dose range, with mean values ranging from 29.4 to 31.4 hours. CL/F values decreased with increasing dose from 20 to 60 mg, with values of 1130 L/h and 937 L/h, respectively, then decreased above 60 mg and were similar across the 120 to 400 mg dose range, with mean values ranging 452 to 541 L/hr. V_{z}/F values increased markedly with increasing dose from 20 mg to 60 mg, with mean values of 1380 L and 6790 L, respectively; V_{z}/F increased above 60 mg to a mean of 12,300 L at 120 mg, then increased further at 200 mg and 400 mg, for which comparable mean values of 18,100 and 20,400 L, respectively, were observed.

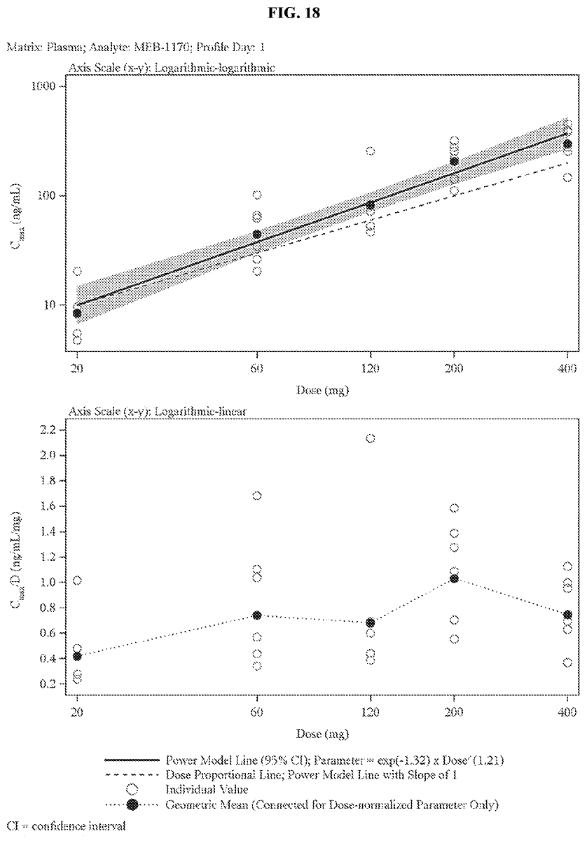

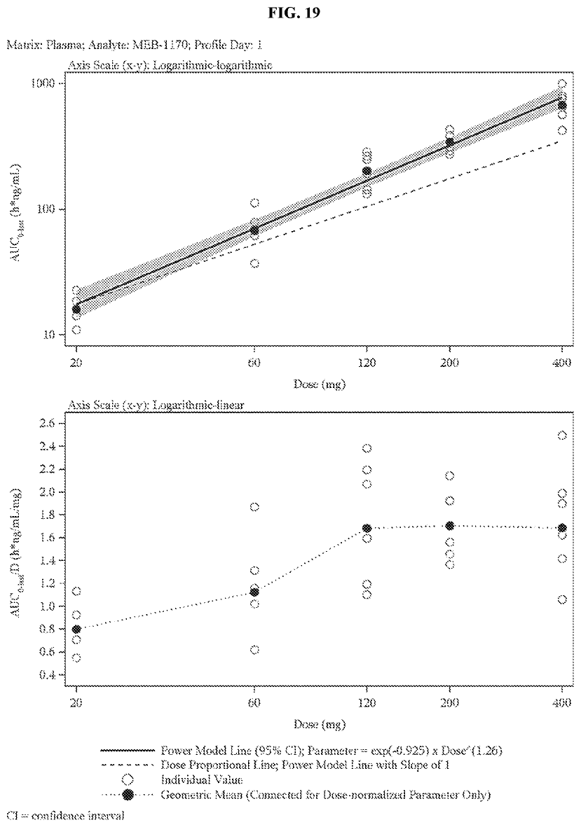

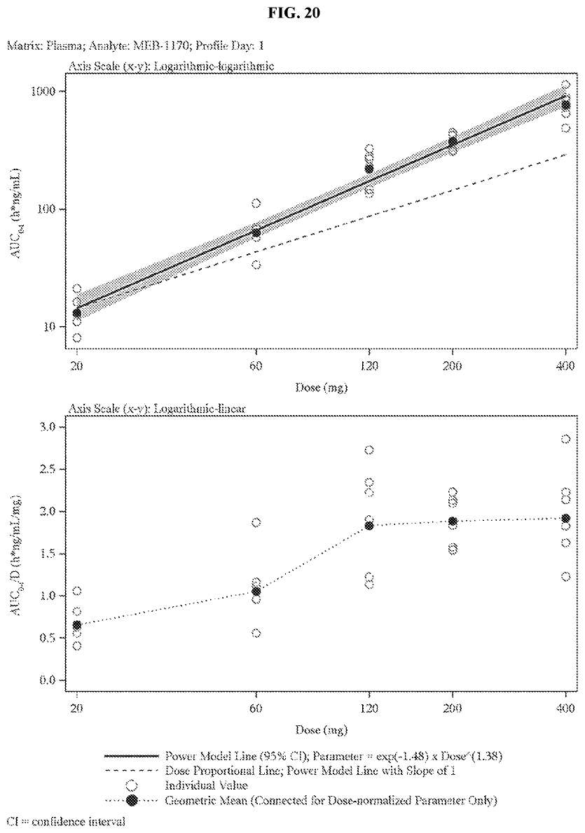

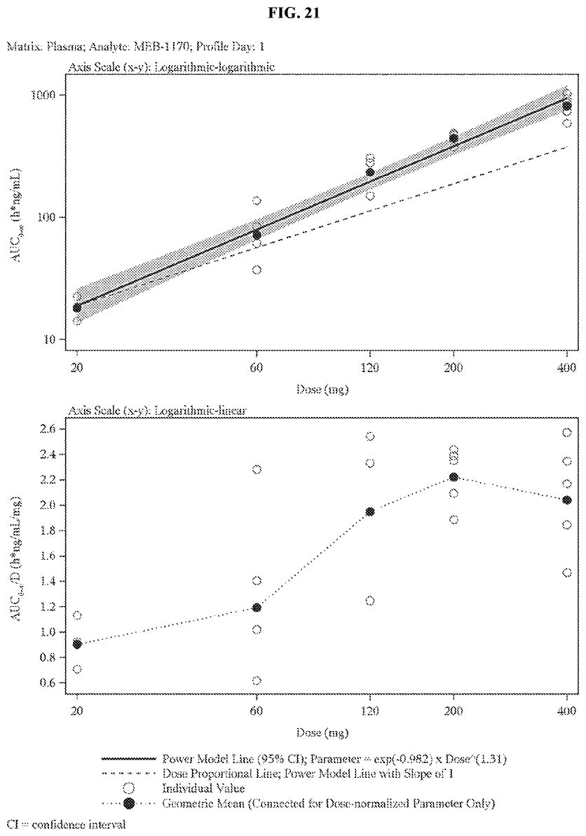

Compound I systemic exposures, as indicated by Cₘₐₓ and AUC values, increased with the escalation in dose. Dose proportionality was evaluated with the power model, based on slope and 95% CIs from linear regression of ln-transformed parameter values vs. In-transformed dose (FIGS. 18-21). Additional dose-proportionality evaluation was performed by visual assessment of dose-normalized parameter values across the dose range. Increases in Cₘₐₓ, AUC₀₋ₜ, AUC₀₋ₗₐₛₜ, and AUC_{0-inf} values were greater than dose proportional, with slope and 95% CIs from the power model of 1.21 (1.00, 1.41), 1.38 (1.26, 1.51), 1.26 (1.14, 1.39), and 1.31 (1.15, 1.46), respectively; the CI limits are outside of the range used as a reference for contextualization purposes only (0.80, 1.25). Based on a comparison of dose-normalized values (FIGS. 18-21), increases in AUC values were greater than dose proportional from 20 to 120 mg, whereas dose proportional increases were observed from 120 mg to 400 mg.

### Food Effect:

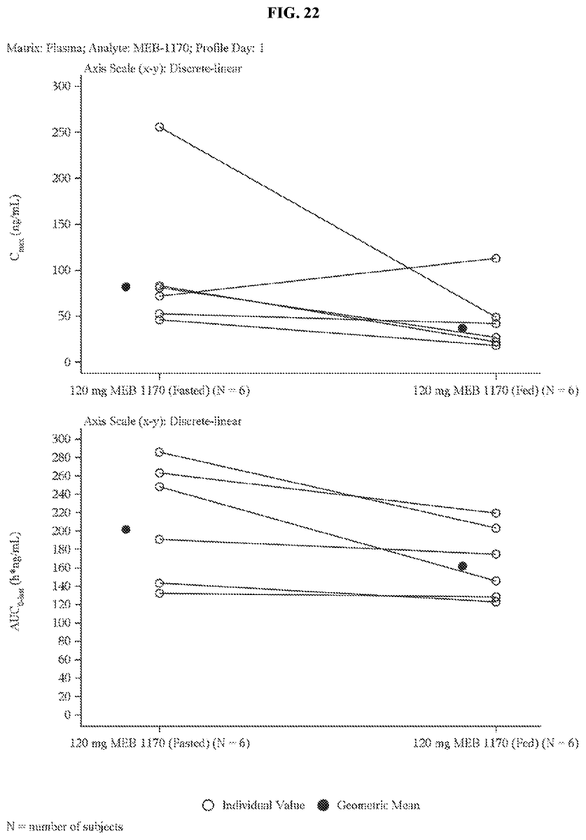

The effect of food on Compound I systemic exposure was evaluated in a crossover assessment of subjects who received a 120 mg dose of MEB-1170 under fasted and fed conditions (FIGS. 16 and 22). The assessment was performed by ANOVA, assessing geometric least squares mean (GLSM) ratios and associated 90% CIs on the ratio for In-transformed Cₘₐₓ and AUC₀₋ₗₐₛₜ values. Following a single oral dose, Compound I systemic exposure under fed conditions were lower than those under fasted conditions, with GLSM ratios and 90% m CIs of 0.450 (0.238, 0.852) and 0.801 (0.688, 0.933) for Cₘₐₓ and AUC₀₋ₗₐₛₜ values, respectively. Without and with food, median Tₘₐₓ values were 2.3 and 2.6 hours, respectively; respective individual Tₘₐₓ values ranged from 2 to 3 and 1 to 6 hours (Table 25).

TABLE 26
Summary of Food Effect for Compound I Exposure

| | | | | | Fed versus Fasted |
|---|---|---|---|---|---|
| | | | | Geometric | Ratio of GLSMs |
| Parameter | Treatment | n | GLSM | CV (%) | (90% CI) |
| Cₘₐₓ | 120 mg DS | 6 | 81.9 | 66.6 | — |
| (ng/mL) | (Fasted) | | | | |
| | 120 mg DS | 6 | 36.9 | 74.4 | 0.450 |
| | (Fed) | | | | (0.238, 0.852) |
| AUC₀₋ₗₐₛₜ | 120 mg DS | 6 | 202 | 33.6 | — |
| (h*ng/mL) | (Fasted) | | | | |
| | 120 mg DS | 6 | 162 | 24.5 | 0.801 |
| | (Fed) | | | | (0.688, 0.933) |

AUC₀₋ₗₐₛₜ = area under the plasma concentration-time curve from zero to 24 hours postdose;
Cₘₐₓ = maximum observed plasma concentration; CV: coefficient of variation;
GLSM = geometric least squares mean; — = not applicable.

### MEB-373

PK parameters are summarized in Table 27.

TABLE 27
Summary of Pharmacokinetic Parameters of Compound
II Following a Single Dose Oral Administration

| | | 20 mg | 60 mg | 120 mg | 120 mg | 200 mg | 400 mg |
|---|---|---|---|---|---|---|---|
| | | DS | DS | DS | DS | DS | DS |
| | | (Fasted) | (Fasted) | (Fasted) | (Fed) | (Fasted) | (Fasted) |
| Parameter | Statistic | (N = 6) | (N = 6) | (N = 6) | (N = 6) | (N = 6) | (N = 6) |
| Cₘₐₓ | Mean | — | NC | NC | — | 7.91 | 12.2 |
| (ng/mL) | SD | — | NC | NC | — | 3.13 | 3.77 |
| Tₘₐₓ (h) | Median | — | NC | NC | — | 2.26 | 2.00 |
| | Min | — | 2.00 | 2.50 | — | 1.00 | 1.50 |
| | Max | — | 2.00 | 2.50 | — | 2.50 | 2.50 |
| Tₗₐₛₜ (h) | Median | — | NC | NC | — | 3.25 | 4.00 |
| | Min | — | 2.50 | 2.50 | — | 2.50 | 2.50 |
| | Max | — | 2.50 | 4.00 | — | 4.00 | 4.00 |
| AUC₀₋ₜ | Mean | — | NC | NC | — | 8.77 | 21.1 |
| (h*ng/mL) | SD | — | NC | NC | — | 4.60 | 8.76 |
| AUC₀₋ₗₐₛₜ | Mean | — | — | — | — | NC | 27.3 |
| (h*ng/mL) | SD | — | — | — | — | NC | 5.20 |
| AUC_{0-inf} | Mean | — | — | — | — | — | 28.7 |
| (h*ng/mL) | SD | — | — | — | — | — | 4.79 |
| t_{1/2} (h) | Mean | — | — | — | — | NC | 0.902 |
| | SD | — | — | — | — | NC | 0.402 |
| MR_{AUC0-t} | Mean | — | NC | NC | — | 0.0231 | 0.0276 |
| | SD | — | NC | NC | — | 0.00991 | 0.00965 |

“—” = not reported (no data); AUC_{0-inf} = area under the plasma concentration-time curve from time 0 extrapolated to infinite time; AUC₀₋ₗₐₛₜ = area under the plasma concentration-time curve from zero to 24 hours postdose; AUC₀₋ₜ = area under the plasma concentration-time curve from zero to the time of the last quantifiable concentration; Cₘₐₓ = maximum observed plasma concentration; Max = maximum; Min = minimum; MR_{AUC0-t} = metabolite ratio (MEB-373/MEB1170 AUC₀₋ₜ values); NC = not calculated (n ≤ 2); SD = standard deviation; t_{1/2} = terminal plasma half-life; Tₘₐₓ = time to achieve maximum plasma concentration; Tₗₐₛₜ = time of last measurable concentration.
Note:
Tₘₐₓ and Tₗₐₛₜ values are presented as median and other parameters are presented as arithmetic mean and SD.

Compound II concentrations in samples from most subjects at doses of 20, 60, and 120 (fed) mg were below the limit of assay quantitation; therefore, only limited PK parameters could be determined in these dose groups.

Following a single oral administration of D in healthy normal subjects, the mean plasma t_{1/2} value for Compound II was 0.902 hours in the 400 mg dose group (n=5); a t_{1/2} value of 2.00 hours was reported for one individual at 200 mg, whereas t_{1/2} could not be determined for any other subjects across groups.

Compound II systemic exposures, as indicated by Cₘₐₓ and AUC values, increased with the escalation in dose. ANOVA performed on dose-normalized parameter values indicated that increases in Compound II Cₘₐₓ and AUC₀₋ₜ values were approximately dose proportional from 200 mg to 400 mg, with pairwise 2-sided P-values of 0.3287 and 0.5356, respectively.

Compound II systemic exposures were very low relative to Compound I. Individual AUC₀₋ₜ MEB-373/MEB-1170 ratios ranged from 0.0093 to 0.037 across the Compound I dose range evaluated.

Terminal t_{1/2} values for Compound I after administration in the fasted state increased with increasing dose from 20 mg to 120 mg, but were similar across the 120 to 400 mg dose range. Without wishing to be limited by any theory, this is likely due to the fact that Compound I was measurable for longer periods of time at higher doses, which revealed a flatter terminal exponential phase that was not evident at the two lowest doses (FIG. 15). This phenomenon had a similar influence on AUC, CL/F, and Vz/F values. CL/F values decreased with increasing dose from 20 mg to 120 mg and were similar across the 120 to 400 mg dose range. V_{z}/F values increased markedly with increasing dose from 20 mg through 200 mg and were similar at 200 mg and 400 mg. These observations are consistent with the results of dose proportionality assessments, particularly for AUC. Increases in Cₘₐₓ, AUC₀₋ₜ, AUC₀₋ₗₐₛₜ, and AUC_{0-inf} values were greater than dose proportional based on the power model. However, a visual comparison of dose-normalized values suggested that the greater than dose proportional increase in these parameters across the full dose range is primarily due to the lower exposures observed for the truncated concentration-time profiles at 20 mg and 60 mg, and that increases in AUC values from 120 to 400 mg are essentially dose proportional.

Plasma exposures for Compound I were assessed under fasted and fed conditions in a crossover fashion at the 120 mg dose. Peak concentrations were reduced approximately 55% by food, whereas a more modest reduction of approximately 20% was observed for AUC₀₋ₗₐₛₜ. Therefore, administration of Compound I with food significantly reduced the rate of absorption and reduced (to a lesser extent) the overall extent of absorption.

Compound I was rapidly absorbed following oral administration, with median tₘₐₓ values of 1.5 to 2.5 hours observed across dose levels under fasted conditions. Exposures (Cₘₐₓ and AUC) of Compound I increased greater than dose proportionally across the dose range from 20 mg to 120 mg, but increases in exposure from 120 mg to 400 mg were generally proportional.

Compound II, a predominant metabolite of Compound I in liver microsomes and hepatocytes in vitro, was present at minimal levels in human plasma. Increases in Compound II Cₘₐₓ and AUC₀₋ₜ values were approximately dose proportional from 200 to 400 mg. Individual plasma metabolite-to-parent AUC ratios ranged from 0.0093 to 0.037 across the Compound I dose range.

Administration of MEB-1170 at a dose of 120 mg following a meal led to a notable decrease in the rate, but a minimal reduction in the extent of absorption.

### Safety Evaluation:

This was a single-ascending dose study, and thus each subject was to receive only a single dose of study medication. However, the food-effect assessment included two treatment periods, in which subjects were treated in a fasted state, and then, after a washout, treated in a fed state.

A total of 30 subjects received at least one dose of Drug Substance, while 6 of those subjects received two doses of Drug Substance. Ten subjects received only placebo.

Compound I was found to be well-tolerated as a single dose ranging from 20 mg up to 400 mg. There were no observed effects on any body system/system organ class, no dose-response on TEAEs, clinical laboratories, ECGs, vital signs, and sedation. Further, there were no system opiate-associated effects noted. This safety/tolerability profile supports further study of Compound I at higher doses and in a multiple dose setting.

### Example 5: Treatment Period

This example comprises a multiple ascending dose (MAD), sequential cohort study. This part is initiated after the first three SAD cohorts has been fully evaluated for safety and tolerability and the safety review committee has concluded that the MAD portion may commence. Up to 32 subjects are studied in 4 cohorts (Cohorts B1 to B4), each cohort consisting of 8 subjects.

In each of Cohorts B1 to B4, 8 subjects receive Drug Substance and 2 receive placebo. Once daily dosing occurs on Days 1 to 7, inclusive, for all subjects. Each subject participates in 1 treatment period only, residing at the CRU from the evening of Day −1 (the day before first dose) until the morning of Day 9 (48 hours after the final dose, given on the morning of Day 7). All subjects return for a post-study visit on Day 15 (±2 days) for a final safety assessment. Table 28 provides the dose levels.

TABLE 28
Dose Levels - Multiple Dosing

| | | | Total Dose | | | |
|---|---|---|---|---|---|---|
| | Planned Daily | Number | (mg) Drug | | Number of Subjects | |
| | Dose of Drug | of Days | Substance | | | Drug |
| Cohort | Substance | Dosed | (or Placebo) | | Placebo | Substance |
| B1 | 60 mg | 7 | 420 | mg | 2 | 6 |
| | Fasting | | | | | |
| B2 | 100 mg | 7 | 700 | mg | 2 | 6 |
| | Fasting | | | | | |
| B3 | 200 mg | 7 | 1400 | mg | 2 | 6 |
| | Fasting | | | | | |
| B4 | 300 mg or | 7 | 2100 or | | 2 | 6 |
| | 400 mg | | 2800 mg | | | |
| | ^{a} Fasting | | | | | |

Note:
Further Cohorts may be added as the data evolve.
^{a} If 200 mg dose is well-tolerated, dose have final escalation to 400 mg. If 400 mg dose is not well-tolerated, a 300 mg dose group can be tested.

For all cohorts, morning doses are administered after completing an overnight fast of at least 8 hours. Standard breakfast is available approximately 90 minutes following morning dosing. Evening doses are administered 12 hours after the morning dose and at least 1 hour after completing the evening meal. However, dosing and food considerations (fed or fasted) for the MAD portion of the study can change pending outcomes from the SAD and food-effect assessments.

### ENUMERATED EMBODIMENTS

The following exemplary embodiments are provided, the numbering of which is not to be construed as designating levels of importance: The disclosures of each and every patent, patent application, and publication cited herein are hereby incorporated herein by reference in their entirety.
Embodiment 1. A pharmaceutical composition comprising: at least one pharmaceutically acceptable carrier and an amount of 3-(3S,4R)-3-((dimethylamino)methyl)-4-hydroxy-1-(2-(thiophen-3-yl)ethyl)piperidin-4-yl-benzamide (Compound I) selected from: 20 mg, 40 mg, 60 mg, 80 mg, 100 mg, 120 mg, 140 mg, 160 mg, 180 mg, 200 mg, 220 mg, 240 mg, 260 mg, 280 mg, 300 mg, 320 mg, 340 mg, 360 mg, 380 mg, and 400 mg.
Embodiment 2. The pharmaceutical composition of Embodiment 1, which is formulated for oral administration.
Embodiment 3. The pharmaceutical composition of any one of Embodiments 1-2, which is formulated for immediate release.
Embodiment 4. The pharmaceutical composition of any one of Embodiments 1-2, which is formulated for sustained release.
Embodiment 5. The pharmaceutical composition of any one of Embodiments 1-2, which is formulated for delayed release.
Embodiment 6. The pharmaceutical composition of any one of Embodiments 1-2, which is formulated for pulsative release.
Embodiment 7. 3-(3S,4R)-3-((dimethylamino)methyl)-4-hydroxy-1-(2-(thiophen-3-yl)ethyl)piperidin-4-yl-benzamide (Compound I) diphosphate crystalline solid.

In certain embodiments, the solid has a X-ray powder diffraction spectrum comprising a 2θ value (in degrees) of about 5.02. In certain embodiments, the solid has a X-ray powder diffraction spectrum comprising a 2θ value (in degrees) of about 7.04. In certain embodiments, the loid has a X-ray powder diffraction spectrum comprising a 2θ value (in degrees) of about 15.12. In certain embodiments, the solid has a X-ray powder diffraction spectrum comprising a 2θ value (in degrees) of about 20.25. In certain embodiments, the solid has a X-ray powder diffraction spectrum comprising a 2θ value (in degrees) of about 20.88. In certain embodiments, the solid has a X-ray powder diffraction spectrum comprising a 2θ value (in degrees) of about 23.00. In certain embodiments, the solid has a X-ray powder diffraction spectrum comprising 2θ values (in degrees) of about: 5.02, 7.04, 15.12, 20.25, 20.88, and 23.0.

In certain embodiments, the solid has a X-ray powder diffraction spectrum comprising a 2θ value (in degrees) of about 4.98. In certain embodiments, the solid has a X-ray powder diffraction spectrum comprising a 2θ value (in degrees) of about 6.99. In certain embodiments, the loid has a X-ray powder diffraction spectrum comprising a 2θ value (in degrees) of about 15.06. In certain embodiments, the solid has a X-ray powder diffraction spectrum comprising a 2θ value (in degrees) of about 20.13. In certain embodiments, the solid has a X-ray powder diffraction spectrum comprising a 2θ value (in degrees) of about 20.72. In certain embodiments, the solid has a X-ray powder diffraction spectrum comprising a 2θ value (in degrees) of about 22.90. In certain embodiments, the solid has a X-ray powder diffraction spectrum comprising 2θ values (in degrees) of about: 4.98, 6.99, 15.06, 20.13, 20.72, and 22.90.
Embodiment 8. The solid of Embodiment 7, wherein the X-ray powder diffraction spectrum further comprises at least one additional 2θ value (in degrees).

In certain embodiments, the solid has a X-ray powder diffraction spectrum comprising a 2θ value (in degrees) of about 10.10. In certain embodiments, the solid has a X-ray powder diffraction spectrum comprising a 2θ value (in degrees) of about 14.08. In certain embodiments, the solid has a X-ray powder diffraction spectrum comprising a 2θ value (in degrees) of about 15.83. In certain embodiments, the solid has a X-ray powder diffraction spectrum comprising a 2θ value (in degrees) of about 18.78. In certain embodiments, the solid has a X-ray powder diffraction spectrum comprising a 2θ value (in degrees) of about 10.10, 14.08, 15.83, and 18.78.

In certain embodiments, the solid has a X-ray powder diffraction spectrum comprising a 2θ value (in degrees) of about 10.07. In certain embodiments, the solid has a X-ray powder diffraction spectrum comprising a 2θ value (in degrees) of about 13.99. In certain embodiments, the solid has a X-ray powder diffraction spectrum comprising a 2θ value (in degrees) of about 15.74. In certain embodiments, the solid has a X-ray powder diffraction spectrum comprising a 2θ value (in degrees) of about 18.64. In certain embodiments, the solid has a X-ray powder diffraction spectrum comprising a 2θ value (in degrees) of about 10.07, 13.99, 15.74, and 18.64.
Embodiment 9. The solid of any one of Embodiments 7-8, which is a hydrate.
Embodiment 10. The solid of any one of Embodiments 7-9, which is a monohydrate.
Embodiment 11. 3-(3S,4R)-3-((dimethylamino)methyl)-4-hydroxy-1-(2-(thiophen-3-yl)ethyl)piperidin-4-yl-benzamide (Compound I) diphosphate crystalline solid, with a X-ray powder diffraction spectrum substantially equal to that of FIG. 9A and/or FIG. 12A.
Embodiment 12. The solid of any one of Embodiments 7-11, further characterized by a Differential Scanning Calorimetry (DSC) thermogram having a single maximum value at about 232° C.
Embodiment 13. The solid of any one of Embodiments 7-12, wherein the DSC measurement is performed by heating the solid from 25° C. at 10° C./min to at least 30° C. above the solid's melting point.
Embodiment 14. A pharmaceutical composition comprising at least one pharmaceutically acceptable carrier and the solid of any one of Embodiments 7-13.
Embodiment 15. The pharmaceutical composition of Embodiment 14, which is in solid dosage form for oral administration.
Embodiment 16. The pharmaceutical composition of any one of Embodiments 14-15, which is part of a tablet, dragee, drop, suppository, capsule, caplet, and/or gelcap.
Embodiment 17. The pharmaceutical composition of any one of Embodiments 14-16, which comprises about 20 mg, 40 mg, 60 mg, 80 mg, 100 mg, 120 mg, 140 mg, 160 mg, 180 mg, 200 mg, 220 mg, 240 mg, 260 mg, 280 mg, 300 mg. 320 mg, 340 mg, 360 mg, 380 mg, or 400 mg of Compound I diphosphate, or any multiples or combinations thereof.
Embodiment 18. A method of treating, ameliorating, and/or preventing pain in a subject, the method comprising administering to the subject a daily amount of 3-(3S,4R)-3-((dimethylamino)methyl)-4-hydroxy-1-(2-(thiophen-3-yl)ethyl)piperidin-4-yl-benzamide (Compound I) selected from: 20 mg, 40 mg, 60 mg, 80 mg, 100 mg, 120 mg, 140 mg, 160 mg, 180 mg, 200 mg, 220 mg. 240 mg, 260 mg. 280 mg, 300 mg. 320 mg, 340 mg, 360 mg, 380 mg, and 400 mg. In certain embodiments, the Compound I is the solid of any one of Embodiments 7-13. In certain embodiments, the Compound I is formulated as part of the pharmaceutical composition of any one of Embodiments 14-17.
Embodiment 19. The method of Embodiment 18, wherein the daily amount of Compound I is administered as a pharmaceutical composition.
Embodiment 20. The method of any one of Embodiments 18-19, wherein the daily amount of Compound I is administered as a single daily dose to the subject.
Embodiment 21. The method of any one of Embodiments 18-19, wherein the daily amount of Compound I is administered as two identical daily doses to the subject.
Embodiment 22. The method of any one of Embodiments 18-19, wherein the daily amount of Compound I is administered as three identical daily doses to the subject.
Embodiment 23. The method of any one of Embodiments 18-22, wherein the administering takes place when the subject is fasting.
Embodiment 24. The method of Embodiment 23, wherein the subject is fasting for at least 1 hour, 2 hours, 3 hours, 4 hours, 5 hours, 6 hours, 7 hours, 8 hours, 9 hours, 10 hours, 11 hours, 12 hours, 13 hours, 14 hours, or 15 hours before the administering.
Embodiment 25. The method of any one of Embodiments 23-24, wherein the subject is allowed to consume solid food at least 15 min, 30 min, 45 min, 60 min, 1 hour, 1.25 hours, 1.5 hours, 1.75 hours, 2 hours, 2.25 hours, 2.5 hours, 2.75 hours, 3 hours, 3.25 hours, 3.5 hours, 3.5 hours, 3.75 hours, or 4 hours after the administering.
Embodiment 26. The method of any one of Embodiments 18-25, wherein the administering decreases cyclic adenosine monophosphate (cAMP) levels in the subject.
Embodiment 27. The method of any one of Embodiments 18-26, wherein the administering does not significantly induce recruitment, binding to, or association with a β-arrestin.
Embodiment 28. The method of any one of Embodiments 18-27, wherein the administering does not significantly cause at least one side effect selected from the group consisting of tachyphylaxis, respiratory depression, constipation, nausea, emesis, withdrawal, dependence, and addiction.
Embodiment 29. The method of any one of Embodiments 18-28, wherein the pain comprises chronic pain, neuropathic pain, nociceptive pain, hyperalgesia, and/or allodynia.
Embodiment 30. The method of any one of Embodiments 18-29, wherein the Compound I is the only therapeutically effective agent administered to the subject to treat, ameliorate, and/or prevent the pain.
Embodiment 31. The method of any one of Embodiments 18-30, wherein the Compound I is the only therapeutically effective agent administered to the subject in a therapeutically effective amount to treat, ameliorate, and/or prevent the pain.
Embodiment 32. The method of any one of Embodiments 18-32, wherein the subject is a mammal.
Embodiment 33. The method of Embodiment 32, wherein the mammal is human.

While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. A pharmaceutical composition comprising:
at least one pharmaceutically acceptable carrier and
an amount of 3-(3S,4R)-3-((dimethylamino)methyl)-4-hydroxy-1-(2-(thiophen-3-yl)ethyl)piperidin-4-yl-benzamide (Compound I) selected from the group consisting of: 20 mg, 40 mg, 60 mg, 80 mg, 100 mg, 120 mg, 140 mg, 160 mg, 180 mg, 200 mg, 220 mg, 240 mg, 260 mg, 280 mg, 300 mg, 320 mg, 340 mg, 360 mg, 380 mg, and 400 mg.

2. The pharmaceutical composition of claim 1, which is formulated for oral administration.

3. The pharmaceutical composition of claim 1, which is formulated for immediate release, sustained release, delayed release, or pulsative release.

4-6. (canceled)

7. 3-(3S,4R)-3-((dimethylamino)methyl)-4-hydroxy-1-(2-(thiophen-3-yl)ethyl)piperidin-4-yl-benzamide (Compound I) diphosphate crystalline solid, with a X-ray powder diffraction spectrum comprising 2θ values (in degrees) of about: 5.02, 7.04, 15.12, 20.25, 20.88, and 23.0.

8. The solid of claim 7, wherein the X-ray powder diffraction spectrum further comprises at least one 2θ value (in degrees) of about: 10.10, 14.08, 15.83, and 18.78.

9. The solid of claim 7, which is a hydrate.

10. The solid of claim 7, which is a monohydrate.

11. 3-(3S,4R)-3-((dimethylamino)methyl)-4-hydroxy-1-(2-(thiophen-3-yl)ethyl)piperidin-4-yl-benzamide (Compound I) diphosphate crystalline solid, with a X-ray powder diffraction spectrum substantially equal to that of FIG. 9A or FIG. 12A.

12. The solid of claim 7, further characterized by a Differential Scanning Calorimetry (DSC) thermogram having a single maximum value at about 232° C.

13. The solid of claim 7, wherein the DSC measurement is performed by heating the solid from 25° C. at 10° C./min to at least 30° C. above the solid's melting point.

14. A pharmaceutical composition comprising at least one pharmaceutically acceptable carrier and the solid of claim 7.

15. The pharmaceutical composition of claim 14, which is in solid dosage form for oral administration.

16. The pharmaceutical composition of claim 14, which is part of a tablet, dragee, drop, suppository, capsule, caplet, or gelcap.

17. The pharmaceutical composition of claim 14, which comprises about 20 mg, 40 mg, 60 mg, 80 mg, 100 mg, 120 mg, 140 mg, 160 mg, 180 mg, 200 mg, 220 mg, 240 mg, 260 mg, 280 mg, 300 mg, 320 mg, 340 mg, 360 mg, 380 mg, or 400 mg of Compound I diphosphate, or any multiples or combinations thereof.

18. A method of treating, ameliorating, or preventing pain in a subject,
the method comprising administering to the subject a daily amount of 3-(3S,4R)-3-((dimethylamino)methyl)-4-hydroxy-1-(2-(thiophen-3-yl)ethyl)piperidin-4-yl-benzamide (Compound I) selected from the group consisting of: 20 mg, 40 mg, 60 mg, 80 mg, 100 mg, 120 mg, 140 mg, 160 mg, 180 mg, 200 mg, 220 mg, 240 mg, 260 mg, 280 mg, 300 mg, 320 mg, 340 mg, 360 mg, 380 mg, and 400 mg,
optionally wherein the Compound I is the solid of claim 7 or a pharmaceutical composition comprising same.

19. The method of claim 18, wherein the daily amount of Compound I is administered as a pharmaceutical composition.

20. The method of claim 18, wherein the daily amount of Compound I is administered as a single daily dose to the subject, as two identical daily doses to the subject, or as three identical daily doses to the subject.

21-22. (canceled)

23. The method of claim 18, wherein the administering takes place when the subject is fasting.

24. The method of claim 23, wherein at least one the following applies:
(a) the subject is fasting for at least 1 hour, 2 hours, 3 hours, 4 hours, 5 hours, 6 hours, 7 hours, 8 hours, 9 hours, 10 hours, 11 hours, 12 hours, 13 hours, 14 hours, or 15 hours before the administering;
(b) the subject is allowed to consume solid food at least 15 min, 30 min, 45 min, 60 min, 1 hour, 1.25 hours, 1.5 hours, 1.75 hours, 2 hours, 2.25 hours, 2.5 hours, 2.75 hours, 3 hours, 3.25 hours, 3.5 hours, 3.5 hours, 3.75 hours, or 4 hours after the administering.

25. (canceled)

26. The method of claim 18, wherein at least one of the following applies:
(a) the administering decreases cyclic adenosine monophosphate (cAMP) levels in the subject;
(b) the administering does not significantly induce recruitment, binding to, or association with a β-arrestin;
(c) the administering does not significantly cause at least one side effect selected from the group consisting of tachyphylaxis, respiratory depression, constipation, nausea, emesis, withdrawal, dependence, and addiction;
(d) the pain comprises chronic pain, neuropathic pain, nociceptive pain, hyperalgesia, and/or allodynia;
(e) the Compound I is the only therapeutically effective agent administered to the subject to treat, ameliorate, and/or prevent the pain;
(f) the Compound I is the only therapeutically effective agent administered to the subject in a therapeutically effective amount to treat, ameliorate, and/or prevent the pain;
(g) the subject is a mammal:
(h) the subject is a human.

27-33. (canceled)
